(19) United States
(12) Patent Application Publication
(10) Pub. No.: US 2008/0194539 A1
(43) Pub. Date: Aug. 14, 2008

(54) Phenyl Diazepane Carboxamides and Annelated Phenyl Piperazine Carboxamides Containing Oxygen and Used as Dopamine D3 Antagonists

(75) Inventors: Peter Gmeiner, Buckenhof (DE); Harald Hubner, Heroldsbach (DE); Karin Schlotter, Oettingen (DE)

Correspondence Address: HARNESS, DICKEY & PIERCE, P.L.C., P.O. BOX 828, BLOOMFIELD HILLS, MI, 48303, US

(73) Assignee: Schwarz Pharma AG, Monheim (DE)

(21) Appl. No.: 11/794,386

(22) PCT Filed: Dec. 27, 2005
(86) PCT No.: PCT/EP2005/014047

§ 371 Date: Jun. 27, 2007

(30) Foreign Application Priority Data
Dec. 30, 2004 (DE) 10 2004 063 797.0

(51) Int. Cl. A61K 31/553; C07D 333/52; A61K 31/381; C07D 403/02; A61P 13/02; A61P 25/16; A61P 25/18; A61P 25/00; A61K 31/551; C07D 413/02

(52) U.S. Cl. 514/211.09; 549/58; 514/443; 540/567; 514/221; 540/552

(57) ABSTRACT

The invention relates to annelated phenyl piperazine and phenyl diazepane carboxamides of general formula (I) containing oxygen.

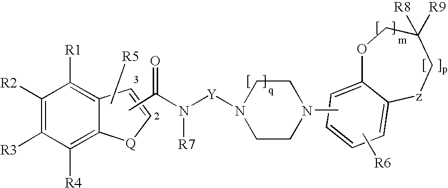

The compounds are suitable for medical use.

Dopamine is an important neurotransmitter of the central nervous system. Dopamine is effective by binding to five different dopamine receptors. As a result of their morphology and the nature of their signal transmission these can be classified as D1-like (D1 and D5) and D2-like (D2-, D3- and D4-receptors) (Neve, K. A. The Dopamine Receptors. Humana Press, 1997). The sub-types of the D2 family in particular have an important part to play in the regulation of central nervous processes. While the D2-receptors are predominantly expressed in the basal ganglions and are involved there in the control and modulation of neuromotor circuits, D3-receptors are mainly found in the mesolimbic system, in which emotional and cognitive processes are controlled. Disturbances in the signal transduction of these receptors lead to a number of neuropathological changes which can sometimes result in serious illnesses. As a result the D3-receptor in particular is a promising target for the development of active substances for the treatment of psychiatric illnesses such as schizophrenia or unipolar depressions, of disturbances of consciousness and for treatment of neurodegenerative diseases such as Parkinson's and the dyskinesia that can occur in the course of long-term therapy, but also for the treatment of drug dependency (Pulvirenti, L. et al. Trends Pharmacol. Sci. 2002, 23, 151-153, Joyce, J. N. Pharmacol. Ther. 2001, 90, 231-259). Here the most D3-receptor-selective bonding profile should be sought. Depending on the intrinsic activity (full agonist, partial agonist, antagonist or inverse agonist) such ligands can have a stimulating, modulating or also inhibiting effect on the pathologically altered dopamine signal transduction system and can thus be used for the treatment of these diseases.

Compounds with an arylpiperazine structure have previously been described as dopamine receptor-active ligands (Robarge, M. J. J. Med. Chem. 2001, 44, 3175-3186). Benzamides and naphthamides with arylpiperazine partial structures are also known as ligands of dopamine receptors (Perrone, R. J. Med. Chem. 1998, 41, 4903-4909; EP 0 779 284 A1). Recently heteroarene amides have also been described as D3-receptor-active compounds (Bettinetti, L. et al. J. Med. Chem. 2002, 45, 4594-4597, Leopoldo, M. et al. J. Med. Chem. 2002, 45, 5727-5735, WO 2004004729 A1). A phenylpiperazinylnaphthamide has also recently been reported on as a selective D3-partial agonist, which demonstrated hopeful activities in the animal model, and which could be used for the treatment of cocaine addiction (Pilla, M. et al. Nature 1999, 400, 371-375). Furthermore, because of the characteristic features of this compound elimination of the serious motor impairments (dyskinesias) caused by long-term treatment of Parkinson's disease with the pharmaceutical preparation L-DOPA can be achieved (Bezard, E. et al. Nature Med. 2003, 9, 762-767). The most recent literature describes the neuro-protective effect of D3-selective partial agonists against MPTP-induced neurone loss in mice as a murine model for Parkinson's disease (Boeckler, F. et al. Biochem. Pharmacol. 2003, 6, 1025-1032).

The structural characteristic shared by many highly affine dopamine receptor ligands concerns a variable substituted phenyl piperazine partial structure, which is linked via a spacer of several carbons in length to an aryl- or heteroarylcarboxamide. Of the range of arylpiperazinylheteroarene carboxamides structure examples with oxygen-, sulphur- or nitrogen-containing heteroarene carboxylic acid components are above all described (ES 2027898; EP 343 961; U.S. Pat. No. 3,646,047; U.S. Pat. No. 3,734,915; WO 2004/024878; Leopoldo, M. et al. J. Med. Chem. 2002, 45, 5727-5735, Bettinetti, L. et al. J. Med. Chem. 2002, 45, 4594-4597; Campiani, G. et al. J. Med. Chem. 2003, 46, 3822-3839; Hackling, A. et al. J. Med. Chem. 2003, 46, 3883-3889; WO 2004004729 A1).

Such compounds comprise an indole, benzothiophene or benzofuran carboxamide component, which is bonded via an aliphatic spacer to an optionally substituted phenyl piperazine.

Alicylic residues and simple functional groups have up until now been described as substituents of the phenyl (Bettinetti, L. et al. J. Med. Chem. 2002, 45, 4594-4597, Chu, W. et al. Bioorg. Med. Chem. 2005, 13, 77-87). In structure-activity investigations with ligands for applied biogene amine receptors, which have various substitution patterns at the phenyl group, it has however been shown that according to the type of substituents and the linking position at the phenyl ring, modulation of the receptor affinity and selectivity and also the intrinsic activity is possible (Heinrich et al. J. Med. Chem. 2004, 47, 4677-4683, Heinrich et al. J. Med. Chem. 2004, 47, 4684-4692, EP0372657).

An aim of the present patent application is to provide new substances with high affinity to dopamine receptors, in particular to the human D3 receptor. Our intensive structure-effect investigations with various dopamine receptor ligands have now surprisingly shown that the dopamine D3 receptor also recognises indole, benzothiophene and benzofuran carboxamides as high affinity ligands, which are linked via the aliphatic spacer described above to an arylpiperazine, in which the aryl component comprises a phenyl ring, which is annulated with a saturated, oxygenated 5-, 6- or 7-link ring and in this way, for example, forms a dihydrobenzofuran, chromane or tetrahydrobenzoxepine. It has also surprisingly been found that the piperazine ring can be exchanged for a diazepane ring, without the affinity of the substances to the human D3 receptor being lost.

In in vitro investigations these compounds showed high affinity and selective binding characteristics at the D3 receptor and remarkable affinity to adrenergic alpha 1 and serotoninergic 5-HT1a receptors. In particular, substances with a simultaneous high affinity for human D3 and the human 5-HT1a receptor have great potential in a number of medical indications.

The compounds according to the invention could constitute valuable therapeutic agents for the treatment of central nervous system disorders, such as schizophrenia or various types of depression, for neuroprotection in neurodegenerative diseases, in addictive disorders, glaucoma, cognitive disorders, restless leg syndrome, attention deficit hyperactive syndrome (ADHS), hyperprolactinemia, hyperprolactinomia and autism, in idiopathic or medically-induced extrapyramidal motor disturbances, such as akathisia, rigor, dystonia and dyskinesias, as well as various disorders of the urinary tract and pain.

The subject-matter of this invention comprises compounds of the general formula I,

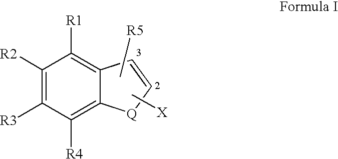

in which:

Q is selected from S, O and NR;

R is selected from among hydrogen, alkyl, phenyl, alkylcarbonyl, phenylalkylcarbonyl, phenylcarbonyl, phenylalkyl and phenylsulfonyl;

R1, R2, R3 and R4 are in each case and independently of one another selected from the group of hydrogen, hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino;

R5 is a group bonded to position 2 or 3 of the bicyclic heteroaryl, selected from among hydrogen, alkyl, halogen, alkoxy and amino and which preferably represents hydrogen or halogen.

X is a group of general formula X1 bonded at position 2 or 3 of the bicyclic heteroaryl in which:

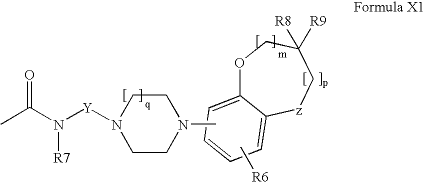

in which:

R6 is selected from the group of hydrogen, hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino;

R7 is hydrogen, alkyl or phenylalkyl;

Y is an unbranched, saturated or unsaturated hydrocarbon chain with 2-5 carbon atoms;

m and p are in each case and independently of one another 0, 1 or 2, wherein the sum of m and p is a maximum of 2; the sum of m and p is preferably 1 or 2, particularly preferably 2;

q is 1 or 2;

Z is CH₂, NH or O and Z is preferably CH₂ or O;

R8 and R9 are in each case and independently of one another selected from among hydrogen, alkyl and phenyl or together form an oxo-group;

in the form of the free base, their physiologically acceptable salts and possible enantiomers and diastereomers.

In a preferred embodiment of the invention the substituents R1, R2, R3, R4 and R6 in the compounds according to the invention of general formulae I to VII (formulae II-VII as specified in more detail below) are selected from the group of hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups, and the C1-C6 alkyl is unsubstituted or hydroxy substituted;

C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups and the C1-C6 alkyl is unsubstituted or hydroxy substituted; C1-C6 alkylaminosulfonyl, in particular methylaminosulfonyl and C1-C6 alkylsulfonylamino; in particular methanesulfonylamino.

X preferably represents a group of general formula X2

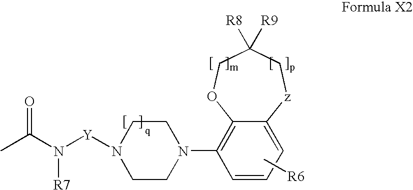

in which R6, R7, R8, R9, m, p, q, Y and Z have the meaning as described in more detail above.

In a preferred embodiment of the invention in formula X1 or X2, R7 represents a hydrogen atom.

In another embodiment of the invention in formula X1 or X2, R6 represents a hydrogen atom.

In another embodiment of the invention in formula X1 or X2, R6 and R7 in each case both represent a hydrogen atom.

In an embodiment of the invention in formula X1 or X2, R8 and R9 in each case both represent a hydrogen atom.

In another embodiment of the invention, R8 and R9 together represent an oxo-group, in particular if Z stands for NH. In this case p preferably has the value 0.

In a preferred embodiment of the invention in formula X1 or X2 R6, R7, R8 and R9 in each case represent hydrogen.

In another preferred embodiment of the invention in formula X1 or X2 R6, R7, R8 and R9 in each case represent a hydrogen atom and Y a saturated, unbranched carbon chain with 2-5 and preferably with 4 or 5 carbons.

In another preferred embodiment of the invention R1, R4, R5, R6 and R7 in each case represent a hydrogen atom and Y a saturated, unbranched carbon chain with 2-5 and preferably with 4 or 5 carbons.

In a preferred embodiment of the invention in the compounds according to the invention Y is a chain of formula —(CH₂)ₙ— with n=2, 3, 4 or 5, most particularly preferably with n=3, 4 or 5, in particular with n=4 or 5.

In an embodiment of the invention in formula X1 or X2 Z represents the group CH₂. In an embodiment of the invention in formula X1 or X2 Z represents an O or a CH₂ group. In another embodiment of the invention Z is an NH group.

In a preferred embodiment of the invention in formula X1 or X2 q represents the value 1. In another preferred embodiment of the invention in formula X1 or X2 q represents the value 2.

In another preferred embodiment of the invention in formula X1 or X2 R1, R4, R5, R6 and R7 in each case represent a hydrogen atom, Y is a saturated, unbranched carbon chain with 3-5 carbons, m and p are in each case 0 and Z is CH₂ or oxygen.

In another preferred embodiment of the invention the following applies:
Q is selected from S, O or NH;
R1 and R4 are H;
R5 is H or halogen;
R2 and R3 are selected from the group comprising hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups, and wherein the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkylaminosulfonyl in particular methylaminosulfonyl and C1-C6 alylsulfonylamino; in particular methanesulfonylamino;
X is a group of formula X1 or X2, for which the following applies:
   R6 represents hydrogen, C1-C6 alkyl, C1-C6 alkoxy or halogen;
   R7 represents hydrogen or C1-C6 alkyl;
   R8 and R9 are hydrogen;
   Z is CH₂ or O;
   Sum of m and p=0, 1 or 2 and particularly preferably 1 or 2;
   q is 1 or 2;
   Y is an unbranched, saturated hydrocarbon chain with 3, 4 or 5 C-atoms.

In a preferred embodiment of the invention the X group with the general formula X1 or X2 is bound in the 2-position to the bicyclic heteroaryl of general formula I and has the general formula II:

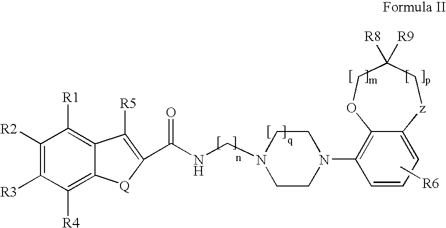

in which R, R1, R2, R3, R4, R5, R6, R8, R9, Q, Z, m, p and q in each case have the meaning as defined in more detail above and in which n has the value 2, 3, 4 or 5.

In a preferred embodiment of the invention R1, R4, R5 and R6 in the compounds of general formula II in each case represent a hydrogen atom.

In a preferred embodiment of the invention in the compounds of general formula II Z represents the group CH₂ and m has the value 0.

In a preferred embodiment of the invention in the compounds of general formula II Z represents a CH₂group or an O.

In an embodiment of the invention in the compounds of general formula II R8 and R9 in each case represent a hydrogen atom.

In another embodiment of the invention R8 and R9 in the compound of general formula II represent an oxo-group, in particular if Z stands for NH. In this case p preferably has the value 0.

In a preferred embodiment of the invention in the compounds of general formula II q has the value 1.

In another preferred embodiment of the invention in formula II R1, R4, R5 and R6 in each case represent a hydrogen atom, n is 2, 3, 4 or 5, m is 0, q is 1 and Z is CH₂ or oxygen, wherein n is preferably 3, 4 or 5 and particularly preferably is 4 or 5.

In another preferred embodiment of the invention in formula II

R1, R4, R6, R8 and R9 in each case represent a hydrogen atom;

R2 and R3 represent hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups and the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups, and wherein the C1-C6 alkyl is unsubstituted or hydroxy substituted; C1-C6 alkylaminosulfonyl in particular methylaminosulfonyl and C1-C6 alylsulfonylamino; in particular methanesulfonylamino

R5 is H or halogen;

n is 3, 4 or 5;

q is 1 or 2;

Z is CH₂ or oxygen;

the sum of m and p is 0, 1 or 2 and particularly preferably 1 or 2;

wherein R2 and R3 are particularly preferably H, halogen, cyano or C2-C6 alkynyl.

Example compounds according to the present invention in accordance with formula II are selected from among
1: N-(4-(4-(2,3-dihydrobenzofuran-7-yl)piperazin-1-yl)butyl)benzo[b]thiophen-2-ylcarbamide,
138: N-(4-(4-(2,3-dihydrobenzofuran-7-yl)piperazin-1-yl)butyl)-3-chlorobenzo[b]thiophen-2-ylcarbamide,
29: N-(4-(4-(2,3-dihydrobenzofuran-7-yl)piperazin-1-yl)butyl)-5-cyanobenzo[b]thiophen-2-ylcarbamide,
139: N-(4-(4-(2,3-dihydrobenzofuran-7-yl)piperazin-1-yl)butyl)-6-ethynylbenzo[b]thiophen-2-ylcarbamide,
30: N-(4-(4-(2,3-dihydrobenzofuran-7-yl)piperazin-1-yl)butyl)benzofuran-2-ylcarbamide,
31: N-(4-(4-(2,3-dihydrobenzofuran-7-yl)piperazin-1-yl)butyl)-5-bromobenzofuran-2-ylcarbamide,
2: N-(4-(4-(2,3-dihydrobenzofuran-7-yl)piperazin-1-yl)butyl)indol-2-ylcarbamide,
32: N-(4-(4-(2,3-dihydrobenzofuran-7-yl)piperazin-1-yl)butyl)-6-cyanoindol-2-ylcarbamide,
8: N-(4-(4-(2,3-dihydrobenzofuran-7-yl)-1,4-diazepan-1-yl)butyl)benzo[b]thiophen-2-ylcarbamide,
142: N-(4-(4-(2,3-dihydrobenzofuran-7-yl)-1,4-diazepan-1-yl)butyl)-3-chlorobenzo[b]thiophen-2-ylcarbamide,
42: N-(4-(4-(2,3-dihydrobenzofuran-7-yl)-1,4-diazepan-1-yl)butyl)-5-cyanobenzo[b]thiophen-2-ylcarbamide,
143: N-(4-(4-(2,3-dihydrobenzofuran-7-yl)-1,4-diazepan-1-yl)butyl)-6-ethynylbenzo[b]thiophen-2-ylcarbamide,
44: N-(4-(4-(2,3-dihydrobenzofuran-7-yl)-1,4-diazepan-1-yl)butyl)benzofuran-2-ylcarbamide,
45: N-(4-(4-(2,3-dihydrobenzofuran-7-yl)-1,4-diazepan-1-yl)butyl)-5-bromobenzofuran-2-ylcarbamide,
47: N-(4-(4-(2,3-dihydrobenzofuran-7-yl)-1,4-diazepan-1-yl)butyl)indol-2-ylcarbamide,
48: N-(4-(4-(2,3-dihydrobenzofuran-7-yl)-1,4-diazepan-1-yl)butyl)-6-cyanoindol-2-ylcarbamide,
3: N-(4-(4-(chroman-8-yl)piperazin-1-yl)butyl)benzo[b]thiophen-2-ylcarbamide,
140: N-(4-(4-(chroman-8-yl)piperazin-1-yl)butyl)-3-chlorobenzo[b]thiophen-2-ylcarbamide,
33: N-(4-(4-(chroman-8-yl)piperazin-1-yl)butyl)-5-cyanobenzo[b]thiophen-2-ylcarbamide,
141: N-(4-(4-(chroman-8-yl)piperazin-1-yl)butyl)-6-ethynylbenzo[b]thiophen-2-ylcarbamide,
34: N-(4-(4-(chroman-8-yl)piperazin-1-yl)butyl)benzofuran-2-ylcarbamide,
35: N-(4-(4-(chroman-8-yl)piperazin-1-yl)butyl)-5-bromobenzofuran-2-ylcarbamide,
4: N-(4-(4-(chroman-8-yl)piperazin-1-yl)butyl)indol-2-ylcarbamide,
37: N-(4-(4-(chroman-8-yl)piperazin-1-yl)butyl)-6-cyanoindol-2-ylcarbamide,
10: N-(4-(4-(chroman-8-yl)-1,4-diazepan-1-yl)butyl)benzo[b]thiophen-2-ylcarbamide,
144: N-(4-(4-(chroman-8-yl)-1,4-diazepan-1-yl)butyl)-3-chlorobenzo[b]thiophen-2-ylcarbamide,
50: N-(4-(4-(chroman-8-yl)-1,4-diazepan-1-yl)butyl)-5-cyanobenzo[b]thiophen-2-ylcarbamide,
145: N-(4-(4-(chroman-8-yl)-1,4-diazepan-1-yl)butyl)-6-ethynylbenzo[b]thiophen-2-ylcarbamide,
11: N-(4-(4-(chroman-8-yl)-1,4-diazepan-1-yl)butyl)benzofuran-2-ylcarbamide,
52: N-(4-(4-(chroman-8-yl)-1,4-diazepan-1-yl)butyl)-5-bromobenzofuran-2-ylcarbamide,
54-N-(4-(4-(chroman-8-yl)-1,4-diazepan-1-yl)butyl)indol-2-ylcarbamide,
55: N-(4-(4-(chroman-8-yl)-1,4-diazepan-1-yl)butyl)-6-cyanoindol-2-ylcarbamide,
12: N-(4-(4-(2,3,4,5-tetrahydrobenzo[b]oxepin-9-yl)piperazin-1-yl)butyl)benzo[b]thiophen-2-ylcarbamide,
136: N-(4-(4-(2,3,4,5-tetrahydrobenzo[b]oxepin-9-yl)piperazin-1-yl)butyl)-3-chlorobenzo[b]thiophen-2-ylcarbamide,
13: N-(4-(4-(2,3,4,5-tetrahydrobenzo[b]oxepin-9-yl)piperazin-1-yl)butyl)-5-cyanobenzo[b]thiophen-2-ylcarbamide,
137: N-(4-(4-(2,3,4,5-tetrahydrobenzo[b]oxepin-9-yl)piperazin-1-yl)butyl)-6-ethynylbenzo[b]thiophen-2-ylcarbamide,
14: N-(4-(4-(2,3,4,5-tetrahydrobenzo[b]oxepin-9-yl)piperazin-1-yl)butyl)benzofuran-2-ylcarbamide,
15: N-(4-(4-(2,3,4,5-tetrahydrobenzo[b]oxepin-9-yl)piperazin-1-yl)butyl)-5-bromobenzofuran-2-ylcarbamide,
16: N-(4-(4-(2,3,4,5-tetrahydrobenzo[b]oxepin-9-yl)piperazin-1-yl)butyl)indol-2-ylcarbamide,
17: N-(4-(4-(2,3,4,5-tetrahydrobenzo[b]oxepin-9-yl)piperazin-1-yl)butyl)-6-cyanoindol-2-ylcarbamide,
18: N-(4-(4-(2,3,4,5-tetrahydrobenzo[b]oxepin-9-yl)-1,4-diazepan-1-yl)butyl)benzo[b]thiophen-2-ylcarbamide,
146: N-(4-(4-(2,3,4,5-tetrahydrobenzo[b]oxepin-9-yl)-1,4-diazepan-1-yl)butyl)-3-chlorobenzo[b]thiophen-2-ylcarbamide,
57: N-(4-(4-(2,3,4,5-tetrahydrobenzo[b]oxepin-9-yl)-1,4-diazepan-1-yl)butyl)-5-cyanobenzo[b]thiophen-2-ylcarbamide,
147: N-(4-(4-(2,3,4,5-tetrahydrobenzo[b]oxepin-9-yl)-1,4-diazepan-1-yl)butyl)-6-ethynylbenzo[b]thiophen-2-ylcarbamide,
19: N-(4-(4-(2,3,4,5-tetrahydrobenzo[b]oxepin-9-yl)-1,4-diazepan-1-yl)butyl)benzofuran-2-ylcarbamide,
59: N-(4-(4-(2,3,4,5-tetrahydrobenzo[b]oxepin-9-yl)-1,4-diazepan-1-yl)butyl)-5-bromobenzofuran-2-ylcarbamide,
61: N-(4-(4-(2,3,4,5-tetrahydrobenzo[b]oxepin-9-yl)-1,4-diazepan-1-yl)butyl)indol-2-ylcarbamide,
62: N-(4-(4-(2,3,4,5-tetrahydrobenzo[b]oxepin-9-yl)-1,4-diazepan-1-yl)butyl)-6-cyanoindol-2-ylcarbamide,
24: N-(4-(4-(benzo[1,3]dioxol-4-yl)piperazin-1-yl)butyl)benzo[b]thiophen-2-ylcarbamide,
148: N-(4-(4-(benzo[1,3]dioxol-4-yl)piperazin-1-yl)butyl)-3-chlorobenzo[b]thiophen-2-ylcarbamide,
64: N-(4-(4-(benzo[1,3]dioxol-4-yl)piperazin-1-yl)butyl)-5-cyanobenzo[b]thiophen-2-ylcarbamide,
149: N-(4-(4-(benzo[1,3]dioxol-4-yl)piperazin-1-yl)butyl)-6-ethynylbenzo[b]thiophen-2-ylcarbamide,
66: N-(4-(4-(benzo[1,3]dioxol-4-yl)piperazin-1-yl)butyl)benzofuran-2-ylcarbamide,
67: N-(4-(4-(benzo[1,3]dioxol-4-yl)piperazin-1-yl)butyl)-5-bromobenzofuran-2-ylcarbamide,
69: N-(4-(4-(benzo[1,3]dioxol-4-yl)piperazin-1-yl)butyl)indol-2-ylcarbamide,
70: N-(4-(4-(benzo[1,3]dioxol-4-yl)piperazin-1-yl)butyl)-6-cyanoindol-2-ylcarbamide, and
158: N-(4-(4-(6-chloro-3-oxo-3,4-dihydro-2H-benzo[1,4]oxazin-8-yl)piperazin-1-yl)butyl)benzo[b]thiophen-2-ylcarbamide.

In a preferred embodiment of the invention the X group with the general formula X1 or X2 is bound in the 3-position to the bicyclic heteroaryl of general formula I and has the general formula III:

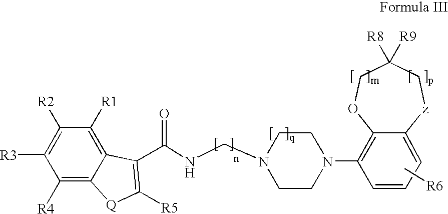

in which R, R1, R2, R3, R4, R5, R6, R8, R9, Q, Z, m, p and q in each case have the meaning as defined in more detail above and in which n has the value 2, 3, 4 or 5.

In preferred embodiments R, R1, R2, R3, R4, R5, R6, R8, R9, Q, Z, n, m, p and q have the meaning as described above for the preferred compounds of formula II, wherein in preferred embodiments, given as examples, of formula III the following applies: Q=O, S, NH; R1, R4, R5, R6, R8 and R9=H; R2 and R3=hydrogen, halogen, cyano or C2-C6 alkynyl; n=4; q=1, 2; Z=O, CH₂.

Example compounds according to formula III of the present invention are selected from among
5: N-(4-(4-(2,3-dihydrobenzofuran-7-yl)piperazin-1-yl)butyl)benzo[b]thiophen-3-ylcarbamide,
6: N-(4-(4-(2,3-dihydrobenzofuran-7-yl)piperazin-1-yl)butyl)benzofuran-3-ylcarbamide,
7: N-(4-(4-(2,3-dihydrobenzofuran-7-yl)piperazin-1-yl)butyl)indol-3-ylcarbamide,
43: N-(4-(4-(2,3-dihydrobenzofuran-7-yl)-1,4-diazepan-1-yl)butyl)benzo[b]thiophen-3-ylcarbamide,
46: N-(4-(4-(2,3-dihydrobenzofuran-7-yl)-1,4-diazepan-1-yl)butyl)benzofuran-3-ylcarbamide,
49: N-(4-(4-(2,3-dihydrobenzofuran-7-yl)-1,4-diazepan-1-yl)butyl)indol-3-ylcarbamide,
51: N-(4-(4-(chroman-8-yl)piperazin-1-yl)butyl)benzo[b]thiophen-3-ylcarbamide,
36: N-(4-(4-(chroman-8-yl)piperazin-1-yl)butyl)benzofuran-3-ylcarbamide,
38: N-(4-(4-(chroman-8-yl)piperazin-1-yl)butyl)indol-3-ylcarbamide,
9: N-(4-(4-(chroman-8-yl)-1,4-diazepan-1-yl)butyl)benzo[b]thiophen-3-ylcarbamide,
53: N-(4-(4-(chroman-8-yl)-1,4-diazepan-1-yl)butyl)benzofuran-3-ylcarbamide,
56: N-(4-(4-(chroman-8-yl)-1,4-diazepan-1-yl)butyl)indol-3-ylcarbamide,
39: N-(4-(4-(2,3,4,5-tetrahydrobenzo[b]oxepin-9-yl)piperazin-1-yl)butyl)benzo[b]thiophen-3-ylcarbamide,
40: N-(4-(4-(2,3,4,5-tetrahydrobenzo[b]oxepin-9-yl)piperazin-1-yl)butyl)benzofuran-3-ylcarbamide,
41: N-(4-(4-(2,3,4,5-tetrahydrobenzo[b]oxepin-9-yl)piperazin-1-yl)butyl)indol-3-ylcarbamide,
58: N-(4-(4-(2,3,4,5-tetrahydrobenzo[b]oxepin-9-yl)-1,4-diazepan-1-yl)butyl)benzo[b]thiophen-3-ylcarbamide,
60: N-(4-(4-(2,3,4,5-tetrahydrobenzo[b]oxepin-9-yl)-1,4-diazepan-1-yl)butyl)benzofuran-3-ylcarbamide,
63: N-(4-(4-(2,3,4,5-tetrahydrobenzo[b]oxepin-9-yl)-1,4-diazepan-1-yl)butyl)indol-3-ylcarbamide,
65: N-(4-(4-(benzo[1,3]dioxol-4-yl)piperazin-1-yl)butyl)benzo[b]thiophen-3-ylcarbamide,
68: N-(4-(4-(benzo[1,3]dioxol-4-yl)piperazin-1-yl)butyl)benzofuran-3-ylcarbamide, and
71: N-(4-(4-(benzo[1,3]dioxol-4-yl)piperazin-1-yl)butyl)indol-3-ylcarbamide.

Another preferred embodiment of the invention concerns compounds of formula IV, in which the X group with the general formula X1 or X2 is bound in the 2-position to the bicyclic heteroaryl of general formula I:

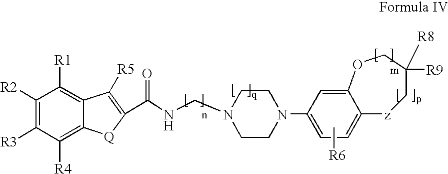

in which R, R1, R2, R3, R4, R5, R6, R8, R9, Q, Z, m, p and q have the meaning as defined in more detail above and in which n has the value 2, 3, 4 or 5.

In preferred embodiments R, R1, R2, R3, R4, R5, R6, R8, R9, Q, Z, n, m, p and q have the meaning as described above for the preferred compounds of formula II and III, wherein in preferred embodiments, given as examples, of formula IV the following applies: Q=S; R1, R2, R3, R4, R5, R6, R8 and R9=H; n=4; q=1,2; Z=O, CH₂.

Example compounds according to formula IV of the present invention are:
20: N-(4-(4-(chroman-7-yl)piperazin-1-yl)butyl)benzo[b]thiophen-2-ylcarbamide,
150: N-(4-(4-(chroman-7-yl)piperazin-1-yl)butyl)-3-chlorobenzo[b]thiophen-2-ylcarbamide,
72: N N-(4-(4-(chroman-7-yl)piperazin-1-yl)butyl)-5-cyanobenzo[b]thiophen-2-ylcarbamide,
151: N-(4-(4-(chroman-7-yl)piperazin-1-yl)butyl)-6-ethynylbenzo[b]thiophen-2-ylcarbamide,
74: N-(4-(4-(chroman-7-yl)piperazin-1-yl)butyl)benzofuran-2-ylcarbamide,
75: N-(4-(4-(chroman-7-yl)piperazin-1-yl)butyl)-5-bromobenzofuran-2-ylcarbamide,
77: N-(4-(4-(chroman-7-yl)piperazin-1-yl)butyl)indol-2-ylcarbamide,
78: N-(4-(4-(chroman-7-yl)piperazin-1-yl)butyl)-6-cyanoindol-2-ylcarbamide,
25: N-(4-(4-(benzo[1,3]dioxol-5-yl)piperazin-1-yl)butyl)benzo[b]thiophen-2-ylcarbamide,
152: N-(4-(4-(benzo[1,3]dioxol-5-yl)piperazin-1-yl)butyl)-3-chlorobenzo[b]thiophen-2-ylcarbamide,
80: N-(4-(4-(benzo[1,3]dioxol-5-yl)piperazin-1-yl)butyl)-5-cyanobenzo[b]thiophen-2-ylcarbamide,
153: N-(4-(4-(benzo[1,3]dioxol-5-yl)piperazin-1-yl)butyl)-6-ethynylbenzo[b]thiophen-2-ylcarbamide,
82: N-(4-(4-(benzo[1,3]dioxol-5-yl)piperazin-1-yl)butyl)benzofuran-2-ylcarbamide,
83: N-(4-(4-(benzo[1,3]dioxol-5-yl)piperazin-1-yl)butyl)-5-bromobenzofuran-2-ylcarbamide,
85: N-(4-(4-(benzo[1,3]dioxol-5-yl)piperazin-1-yl)butyl)indol-2-ylcarbamide,
86: N-(4-(4-(benzo[1,3]dioxol-5-yl)piperazin-1-yl)butyl)-6-cyanoindol-2-ylcarbamide,
26: N-(4-(4-(2,3-dihydrobenzo[1,4]dioxin-6-yl)piperazin-1-yl)butyl)benzo[b]thiophen-2-ylcarbamide,
154: N-(4-(4-(2,3-dihydrobenzo[1,4]dioxin-6-yl)piperazin-1-yl)butyl)-3-chlorobenzo[b]thiophen-2-ylcarbamide,
88: N-(4-(4-(2,3-dihydrobenzo[1,4]dioxin-6-yl)piperazin-1-yl)butyl)-5-cyanobenzo[b]thiophen-2-ylcarbamide,
155: N-(4-(4-(2,3-dihydrobenzo[1,4]dioxin-6-yl)piperazin-1-yl)butyl)-6-ethynylbenzo[b]thiophen-2-ylcarbamide,
90: N-(4-(4-(2,3-dihydrobenzo[1,4]dioxin-6-yl)piperazin-1-yl)butyl)benzofuran-2-ylcarbamide,
91: N-(4-(4-(2,3-dihydrobenzo[1,4]dioxin-6-yl)piperazin-1-yl)butyl)-5-bromobenzofuran-2-ylcarbamide,
93: N-(4-(4-(2,3-dihydrobenzo[1,4]dioxin-6-yl)piperazin-1-yl)butyl)indol-2-ylcarbamide,
94: N-(4-(4-(2,3-dihydrobenzo[1,4]dioxin-6-yl)piperazin-1-yl)butyl)-6-cyanoindol-2-ylcarbamide,
27: N-(4-(4-(2,3-dihydrobenzo[1,4]dioxin-6-yl)-1,4-diazepan-1-yl)butyl)benzo[b]thiophen-2-ylcarbamide,
104: N-(4-(4-(2,3-dihydrobenzo[1,4]dioxin-6-yl)-1,4-diazepan-1-yl)butyl)-5-cyanobenzo[b]thiophen-2-ylcarbamide,
106: N-(4-(4-(2,3-dihydrobenzo[1,4]dioxin-6-yl)-1,4-diazepan-1-yl)butyl)benzofuran-2-ylcarbamide,
107: N-(4-(4-(2,3-dihydrobenzo[1,4]dioxin-6-yl)-1,4-diazepan-1-yl)butyl)-5-bromobenzofuran-2-ylcarbamide,
109: N-(4-(4-(2,3-dihydrobenzo[1,4]dioxin-6-yl)-1,4-diazepan-1-yl)butyl)indol-2-ylcarbamide,
110: N-(4-(4-(2,3-dihydrobenzo[1,4]dioxin-6-yl)-1,4-diazepan-1-yl)butyl)-6-cyanoindol-2-ylcarbamide,
28: N-(4-(4-(3,4-dihydro-2H-benzo[b][1,4]dioxepin-7-yl)piperazin-1-yl)butyl)benzo[b]thiophen-2-ylcarbamide,
156: N-(4-(4-(3,4-dihydro-2H-benzo[b][1,4]dioxepin-7-yl)piperazin-1-yl)butyl)-3-chlorobenzo[b]thiophen-2-ylcarbamide,
96: N-(4-(4-(3,4-dihydro-2H-benzo[b][1,4]dioxepin-7-yl)piperazin-1-yl)butyl)-5-cyanobenzo[b]thiophen-2-ylcarbamide,
157: N-(4-(4-(3,4-dihydro-2H-benzo[b][1,4]dioxepin-7-yl)piperazin-1-yl)butyl)-6-ethynylbenzo[b]thiophen-2-ylcarbamide,
98: N-(4-(4-(3,4-dihydro-2H-benzo[b][1,4]dioxepin-7-yl)piperazin-1-yl)butyl)benzofuran-2-ylcarbamid,
99: N-(4-(4-(3,4-dihydro-2H-benzo[b][1,4]dioxepin-7-yl)piperazin-1-yl)butyl)-5-bromobenzofuran-2-ylcarbamide,
101: N-(4-(4-(3,4-dihydro-2H-benzo[b][1,4]dioxepin-7-yl)piperazin-1-yl)butyl)indol-2-ylcarbamide, and
102: N-(4-(4-(3,4-dihydro-2H-benzo[b][1,4]dioxepin-7-yl)piperazin-1-yl)butyl)-6-cyanoindol-2-ylcarbamide.

Another preferred embodiment of the invention concerns compounds of formula V, in which the X group with the general formula X1 or X2 is bound in the 3-position to the bicyclic heteroaryl of general formula I:

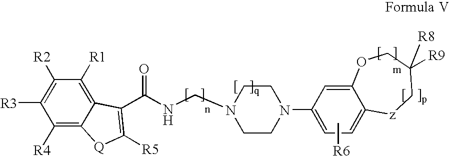

in which R, R1, R2, R3, R4, R5, R6, R8, R9, Q, Z, m, p and q have the meaning as defined in more detail above and in which n has the value 2, 3, 4 or 5.

In preferred embodiments R, R1, R2, R3, R4, R5, R6, R8, R9, Q, Z, n, m, p and q have the meaning as described above for the preferred compounds of formulae II, III and IV.

Example compounds according to formula V of the present invention are:
73: N-(4-(4-(chroman-7-yl)piperazin-1-yl)butyl)benzo[b]thiophen-3-ylcarbamide,
76: N-(4-(4-(chroman-7-yl)piperazin-1-yl)butyl)benzofuran-3-ylcarbamide,
79: N-(4-(4-(chroman-7-yl)piperazin-1-yl)butyl)indol-3-ylcarbamide,
81: N-(4-(4-(benzo[1,3]dioxol-5-yl)piperazin-1-yl)butyl)benzo[b]thiophen-3-ylcarbamide,
84: N-(4-(4-(benzo[1,3]dioxol-5-yl)piperazin-1-yl)butyl)benzofuran-3-ylcarbamide,
87: N-(4-(4-(benzo[1,3]dioxol-5-yl)piperazin-1-yl)butyl)indol-3-ylcarbamide,
89: N-(4-(4-(2,3-dihydrobenzo[1,4]dioxin-6-yl)piperazin-1-yl)butyl)benzo[b]thiophen-3-ylcarbamide,
92: N-(4-(4-(2,3-dihydrobenzo[1,4]dioxin-6-yl)piperazin-1-yl)butyl)benzofuran-3-ylcarbamide,
95: N-(4-(4-(2,3-dihydrobenzo[1,4]dioxin-6-yl)piperazin-1-yl)butyl)indol-3-ylcarbamide,
105: N-(4-(4-(2,3-dihydrobenzo[1,4]dioxin-6-yl)-1,4-diazepan-1-yl)butyl)benzo[b]thiophen-3-ylcarbamide,
108: N-(4-(4-(2,3-dihydrobenzo[1,4]dioxin-6-yl)-1,4-diazepan-1-yl)butyl)benzofuran-3-ylcarbamide,
111: N-(4-(4-(2,3-dihydrobenzo[1,4]dioxin-6-yl)-1,4-diazepan-1-yl)butyl)indol-3-ylcarbamide,
97: N-(4-(4-(3,4-dihydro-2H-benzo[b][1,4]dioxepin-7-yl)piperazin-1-yl)butyl)benzo[b]thiophen-3-ylcarbamide,
100: N-(4-(4-(3,4-dihydro-2H-benzo[b][1,4]dioxepin-7-yl)piperazin-1-yl)butyl)benzofuran-3-ylcarbamide, and
103: N-(4-(4-(3,4-dihydro-2H-benzo[b][1,4]dioxepin-7-yl)piperazin-1-yl)butyl)indol-3-ylcarbamide.

Another embodiment of the invention concerns compounds of formula VI, in which the X group with the general formula X1 or X2 is bound in the 2-position to the bicyclic heteroaryl of general formula I:

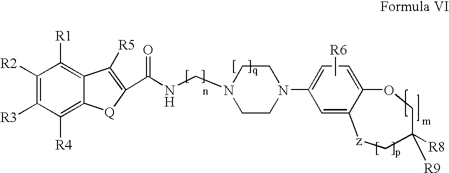

in which R, R1, R2, R3, R4, R5, R6, R8, R9, Q, Z, m, p and q have the meaning as defined in more detail above and in which n has the value 2, 3, 4 or 5.

In preferred embodiments R, R1, R2, R3, R4, R5, R6, R8, R9, Q, Z, n, m, p and q have the meaning as described above for the preferred compounds of formulae II, III and IV.

Example compounds according to formula VI of the present invention are:
21: N-(4-(4-(2,3-dihydrobenzofuran-5-yl)piperazin-1-yl)butyl)benzo[b]thiophen-2-ylcarbamide,
112: N-(4-(4-(2,3-dihydrobenzofuran-5-yl)piperazin-1-yl)butyl)-5-cyanobenzo[b]thiophen-2-ylcarbamide,
114: N-(4-(4-(2,3-dihydrobenzofuran-5-yl)piperazin-1-yl)butyl)benzofuran-2-ylcarbamide,
115: N-(4-(4-(2,3-dihydrobenzofuran-5-yl)piperazin-1-yl)butyl)-5-bromobenzofuran-2-ylcarbamide,
117: N-(4-(4-(2,3-dihydrobenzofuran-5-yl)piperazin-1-yl)butyl)indol-2-ylcarbamide,
118: N-(4-(4-(2,3-dihydrobenzofuran-5-yl)piperazin-1-yl)butyl)-6-cyanoindol-2-ylcarbamide,
22: N-(4-(4-(chroman-6-yl)piperazin-1-yl)butyl)benzo[b]thiophen-2-ylcarbamide,
120: N-(4-(4-(chroman-6-yl)piperazin-1-yl)butyl)-5-cyanobenzo[b]thiophen-2-ylcarbamide,
122: N-(4-(4-(chroman-6-yl)piperazin-1-yl)butyl)benzofuran-2-ylcarbamide,
123: N-(4-(4-(chroman-6-yl)piperazin-1-yl)butyl)-5-bromobenzofuran-2-ylcarbamide,
125: N-(4-(4-(chroman-6-yl)piperazin-1-yl)butylindol-2-ylcarbamide,
126: N-(4-(4-(chroman-6-yl)piperazin-1-yl)butyl)-6-cyanoindol-2-ylcarbamide,
23: N-(4-(4-(2,3,4,5-tetrahydrobenzo[b]oxepin-7-yl)piperazin-1-yl)butyl)benzo[b]thiophen-2-ylcarbamide,
128: N-(4-(4-(2,3,4,5-tetrahydrobenzo[b]oxepin-7-yl)piperazin-1-yl)butyl)-5-cyanobenzo[b]thiophen-2-ylcarbamide,
130: N-(4-(4-(2,3,4,5-tetrahydrobenzo[b]oxepin-7-yl)piperazin-1-yl)butyl)benzofuran-2-ylcarbamide,
131: N-(4-(4-(2,3,4,5-tetrahydrobenzo[b]oxepin-7-yl)piperazin-1-yl)butyl)-5-bromobenzofuran-2-ylcarbamide,
133: N-(4-(4-(2,3,4,5-tetrahydrobenzo[b]oxepin-7-yl)piperazin-1-yl)butyl)indol-2-ylcarbamide, and
134: N-(4-(4-(2,3,4,5-tetrahydrobenzo[b]oxepin-7-yl)piperazin-1-yl)butyl)-6-cyanoindol-2-ylcarbamide.

Another embodiment of the invention concerns compounds of formula VII, in which the X group with the general formula X1 or X2 is bound in the 3-position to the bicyclic heteroaryl of general formula I:

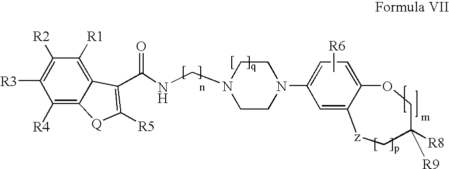

in which R, R1, R2, R3, R4, R5, R6, R8, R9, Q, Z, m, p and q have the meaning as defined in more detail above and in which n has the value 2, 3, 4 or 5.

In preferred embodiments of formula VII R, R1, R2, R3, R4, R5, R6, R8, R9, Q, Z, n, m, p and q have the meaning as described above for the preferred compounds of formulae II, III and IV.

Example compounds according to formula VII of the present invention are selected from among:
113: N-(4-(4-(2,3-dihydrobenzofuran-5-yl)piperazin-1-yl)butyl)benzo[b]thiophen-3-ylcarbamide,
116: N-(4-(4-(2,3-dihydrobenzofuran-5-yl)piperazin-1-yl)butyl)benzofuran-3-ylcarbamide,
119: N-(4-(4-(2,3-dihydrobenzofuran-5-yl)piperazin-1-yl)butyl)indol-3-ylcarbamide,
121: N-(4-(4-(chroman-6-yl)piperazin-1-yl)butyl)benzo[b]thiophen-3-ylcarbamide,
124: N-(4-(4-(chroman-6-yl)piperazin-1-yl)butyl)benzofuran-3-ylcarbamide,
127: N-(4-(4-(chroman-6-yl)piperazin-1-yl)butyl)indol-3-ylcarbamide,
129: N-(4-(4-(2,3,4,5-tetrahydrobenzo[b]oxepin-7-yl)piperazin-1-yl)butyl)benzo[b]thiophen-3-ylcarbamide,
132: N-(4-(4-(2,3,4,5-tetrahydrobenzo[b]oxepin-7-yl)piperazin-1-yl)butyl)benzofuran-3-ylcarbamide, and
135: N-(4-(4-(2,3,4,5-tetrahydrobenzo[b]oxepin-7-yl)piperazin-1-yl)butyl)indol-3-ylcarbamide.

The invention also concerns physiologically acceptable salts of the compounds according to the invention. Examples of such salts are described in the following definitions.

The person skilled in the art will realise that depending on the choice of substituents geometrical isomers and/or optically active compounds can result. In this case both the isomers and racemates and also the respective pure enantiomeric or possibly diastereomeric forms are the subject-matter of the present invention.

The substituents mentioned in the description and in the attached claims include in particular the following groups.

“Alkyl” can be a branched or unbranched alkyl group, which preferably has between 1 and 10 C-atoms, particularly preferably between 1 and 6 C-atoms (“C1-C6 alkyl”) and most particularly preferably 1, 2 or 3 C-atoms. “C1-C6 alkyl” includes, for example, methyl, ethyl, n-propyl, iso-propyl, n-butyl, iso-butyl, s-butyl, t-butyl, n-pentyl, iso-pentyl, neopentyl, t-pentyl, 1-methylbutyl, 2-methylbutyl, 1-ethylpropyl, 1,2-dimethylpropyl and n-hexyl. “Alkyl” can also be cyclical or contain a cyclical component, wherein cycles with 3-7 C-atoms are preferred, e.g. cyclopropyl, cyclobutyl, cyclopentyl, cyclohexyl or cycloheptyl. “Alkyl” is preferably not cyclical and contains no cyclical component. Alkyl groups can also be substituted with one or more substituents, in particular with hydroxy or amine. “Alkyl” is preferably unsubstituted or hydroxy or alkyloxy substituted.

“Alkenyl” and “alkynyl” have at least one double or triple bond, respectively. They can be branched or unbranched and preferably have between 2 and 6 C-atoms. Alkenyls or alkynyls are preferably bonded to the heteroarene- or phenyl ring of the matrix of the compound in such a way that the double or triple bond is conjugated with the aromatic ring. Alkenyl and alkynyl can also be substituted with one or more substituents, preferably with phenyl, wherein the phenyl group then is preferably located at C-atom 2 (if the alkenyl or alkynyl is bonded via C-atom 1 to the heteroarene- or phenyl ring of the matrix). The alkenyls or alkynyls are preferably unsubstituted.

“Alkyloxy” is the —O-alkyl group, in which the alkyl is preferably selected from the groups specified above for “alkyl”. “Alkyloxy” is preferably a C1-C6-alkyloxy group, particularly preferably methoxy.

“Alkylthio” is the —S-alkyl group, in which the alkyl is preferably selected from the groups specified above for “alkyl”. “Alkylthio” is preferably a C1-C6-alkyl-S-group.

“Alkylaminosulfonyl” includes the —SO₂—NH-alkyl and —SO₂—N-dialkyl groups, in which alkyl is preferably selected from the groups specified above for “alkyl”. “Alkyl” in the “alkylaminosulfonyl” is preferably a C1-C6-alkyl group. “Alkylaminosulfonyl” examples include methylaminosulfonyl, N,N-dimethylaminosulfonyl and butylaminosulfonyl.

“Alkylsulfonylamino” is the —NH—SO₂-alkyl group, in which alkyl is preferably selected from the groups specified above for “alkyl”. “Alkylsulfonylamino” is preferably a C1-C6-alkylsulfonylamino group, e.g. methanesulfonylamino.

“Amino” includes primary, secondary or tertiary amines. Secondary or tertiary amines can carry substituents from the group comprising alkyl or phenylalkyl. Alkyl can also carry hydroxy or alkyloxy. Amino is in particular a primary, i.e. exclusively hydrogen substituted, amine.

“Phenyl” is preferably unsubstituted, but can if necessary be independently substituted one or more times, e.g. with alkoxy, alkyl, trifluoromethyl or halogen.

“Phenylalkyl” is the -alkyl-phenyl group, wherein phenyl and alkyl have the meaning as defined above. Phenylalkyl includes for example phenylethyl and benzyl and is preferably benzyl.

“Phenoxy” is the —O-phenyl group, in which phenyl has the meaning as defined in more detail above.

“Alkylcarbonyl” includes the —C(O)-alkyl group, in which alkyl is preferably selected from the groups specified above for “alkyl”, and is particularly preferably —C(O)—C1-C6-alkyl. “Alkylcarbonyl” is preferably acetyl, propionyl or butyryl.

“Phenylcarbonyl” is —C(O)-phenyl, in which phenyl has the meaning as defined in more detail above.

“Phenylalkylcarbonyl” is —C(O)-alkyl-phenyl, in which alkyl and phenyl have the meaning as defined in more detail above.

“Alkyloxycarbonyl” is the —C(O)—O-alkyl group, in which alkyl is preferably selected from the groups specified above for “alkyl”. “Alkoxycarbonyl” is preferably a (C1-C6-alkyl)oxycarbonyl group.

“Phenylalkyloxycarbonyl” is the —C(O)—O-alkyl-phenyl group, in which alkyl and phenyl have the meaning as defined in more detail above

“Halogen” includes fluorine, chlorine, bromine and iodine, and is preferably fluorine, chlorine or bromine.

“Sulfamoyl” includes the —SO₂—NH₂ group.

“Sulfonylamino” includes the —NH—SO₂H group.

“Physiologically acceptable salts” include non-toxic addition salts of a base, in particular a compound of formulae (I) to (VII) in the form of the free base, with organic or inorganic acids. Examples of inorganic acids include HCl, HBr, sulphuric acid and phosphoric acid. Organic acids include acetic acid, propionic acid, pyruvic acid, butyric acid, α-, β- or γ-hydroxbutyric acid, valeric acid, hydroxyvaleric acid, caproic acid, hydroxycaproic acid, caprylic acid, capric acid, lauric acid, myristic acid, palmitic acid, stearic acid, glycolic acid, lactic acid, D-glucuronic acid, L-glucoronic acid, D-galacturonic acid, glycine, benzoic acid, hydroxybenzoic acid, gallic acid, salicylic acid, vanillic acid, coumarinic acid, caffeic acid, hippuric acid, orotic acid, L-tartaric acid, D-tartaric acid, D,L-tartaric acid, meso-tartaric acid, fumaric acid, L-malic acid, D-malic acid, D,L-malic acid, oxalic acid, malonic acid, succinic acid, maleic acid, oxalo-acetic acid, glutaric acid, hydroxyglutaric acid, ketoglutaric acid, adipinic acid, ketoadipinic acid, pimelic acid, glutamic acid, aspartic acid, phthalic acid, propanetricarboxylic acid, citric acid, isocitric acid, methane sulfonic acid, toluene sulfonic acid, benzene sulfonic acid, camphor sulfonic acid, embonic acid and trifluoromethane sulfonic acid.

Compounds of formulae (I) to (VII) as defined, are suitable as pharmaceutical preparations. The compounds according to the invention comprise affine or even high affinity ligands for D3 receptors.

The term “affine D3-ligand” covers compounds which in a radioligand experiment demonstrate binding (see Hübner, H. et al. J. Med. Chem. 2000, 43, 756-762 and the section on “Biological Activity”) to human dopamine D3-receptors with a Ki-value of not more than 500 nM. For “affine” ligands of other receptors the definition applies by analogy.

The term “high affinity D3-ligands” covers compounds which in a radioligand experiment demonstrate binding (see Hübner, H. et al. J. Med. Chem. 2000, 43, 756-762 and the section on “Biological Activity”) to human dopamine D3-receptors with a Ki-value of preferably not more than approximately 30 nM, particularly preferably not more than 3 nM. For “high affinity” ligands of other receptors the definition applies by analogy.

One aspect of the present invention concerns selective D3-ligands. The term “selective D3-ligands” covers compounds that in the radioligand experiment for the D3-receptor, as described in the following section “Biological Activity”, have a Ki value that is lower by a factor of at least 10 than for at least five of the following seven receptors: dopamine receptors D1, D2long, D2short and D4.4, serotonin receptors 5-HT1a and 5-HT2 and alpha 1 adrenoceptor.

Another aspect of the invention concerns highly selective dopamine D3-ligands. The term “highly selective D3-ligands” covers compounds that in the radioligand experiment for the D3-receptor, as described in the following section “Biological Activity”, have a Ki value that is lower by a factor of at least 100 than for at least five of the following seven receptors: dopamine receptors D1, D2long, D2short and D4.4, serotonin receptors 5-HT1a and 5-HT2 and alpha 1 adrenoceptor.

For “affine” or “high affinity” ligands of the 5-HT1a or alpha 1-adrenoceptor, corresponding definitions apply.

D3-ligands can have an agonistic, antagonistic or partial agonistic effect at the D3-receptor. The corresponding intrinsic activities of the compounds according to the invention can be measured in mitogenesis assays, as described in the literature (Hübner, H. et al. J. Med. Chem. 2000, 43,4563-4569 and Löber S., Bioorg. Med. Chem. Lett. 2002, 12.17, 2377-2380). Depending on the pathophysiology of the underlying illness a stronger agonistic, a stronger antagonistic or a partial agonistic activity may be therapeutically desired.

For example, for the treatment of idiopathic Parkinson's disease dopamine modulators with a strong agonistic component are frequently desired, while for the treatment of schizophrenia as a rule pure antagonists are used. Partial D3-agonists, on the other hand, have for example potential in the treatment of L-DOPA-induced dyskinesias.

Finally, some of the substances according to the invention also have significant affinity to other pharmacologically interesting receptors, such as the serotonin receptor, in particular the 5-HT1a-receptor, or the adrenergic alpha-1-receptor.

In place of highly selective dopamine D3-receptor binding, depending on the type of illness to be treated, binding to a further receptor may be desired.

For example, for the treatment of schizophrenia a compound may be attractive which is a high affinity D3-ligand and at the same time an affine or even high affinity 5-HT1a-receptor ligand. In another embodiment of the invention for the treatment of dyskinesias a compound may be desired which apart from D3-modulatory characteristics also has D2-agonistic, alpha1- and/or 5-HT1a-modulatory characteristics.

The present invention therefore allows fine tuning or careful selection of the desired affinity, activity and selectivity in respect of various pharmacologically significant receptors, in particular the dopamine D3-receptors, but also for example in respect of the 5-HT1a-receptor or the D2-receptor.

Also forming the subject-matter of the invention is therefore a pharmaceutical preparation containing one or more of the compounds of general formulae (I) to (VII), or one of the specifically listed compounds as defined above, possibly in the form of a pharmaceutically acceptable salt as well as a pharmaceutically acceptable adjuvant.

The invention also concerns the use of one or more of the compounds of general formulae (I) to (VII), or one of the specifically listed compounds, possibly in the form of a pharmaceutically acceptable salt, for the treatment of the indications mentioned here and the production of a pharmaceutical preparation for the indications mentioned here.

The term “treatment” of an illness covers in this patent application (a) therapy for a pre-existing illness and (b) prevention of an illness that has not yet developed or not yet fully developed, if there is a risk of such an illness occurring.

For the production of pharmaceutical preparations compounds according to the invention are preferably selected which are high affinity D3-ligands. Particularly preferred is the use of selective or even highly selective D3-ligands.

In another embodiment of the invention compounds are selected which are affine or even high affinity also or in particular for the adrenergic alpha-1- or the 5-HT1a-receptor.

The compounds according to the invention have potential in the treatment or prevention of a series of illnesses, which in particular accompany dopamine metabolism or dopaminergic signalling cascade, or possibly serotoninergic signal transmission disorders.

Subject-matter of the invention is therefore the use of a compound according to the invention, as described in this patent application, including the claims and the examples, for the production of a pharmaceutical preparation for the treatment of illnesses which accompany dopamine metabolism and/or dopaminergic signalling cascade disorders.

Also forming the subject-matter of the invention is the use of a compound according to the invention, as described in this patent application, including the claims and the examples, for the production of a pharmaceutical preparation for the treatment of illnesses which accompany an adrenergic signal cascade disorder or result from this and/or which can be treated by the administration of alpha-adrenergic mimetics or inhibitors. Examples of such illnesses are hypertonia or benign prostate hyperplasia.

Also forming the subject-matter of the invention is the use of a compound according to the invention, as described in this patent application, including the claims and the examples, for the production of a pharmaceutical preparation for the treatment of illnesses which accompany serotonin metabolism and/or serotoninergic signal transmission disorders.

Illnesses in whose pathogenesis dopaminergic and/or serotoninergic processes are involved, are in particular illnesses of the central nervous system. Included in the subject-matter of the invention is therefore the use of a compound according to the invention, as described in this patent application, including the claims and examples, for the production of a pharmaceutical preparation for the treatment of central nervous system illnesses.

The term “central nervous system illnesses” covers in this patent application both disorders that have their origin in the central nervous system and whose symptoms are predominantly or exclusively noticeable in the central nervous system, such as psychoses, depressions or cognitive disorders, and illnesses which have their origin in the central nervous system, whose symptoms however at least in part are noticed in other target organs, such as extrapyramidal motor disturbances or hyperprolactinemia.

Examples of central nervous system illnesses which can be treated with the compounds according to the invention are:
(1) psychoses and anxiety disorders, including manias, idiopathic psychoses, schizophrenia, compulsive disorders, panic attacks, phobias, eating disorders, aggressive and autoagressive disorders, stereotypies and other personality disorders;
(2) drug dependency, e.g. cocaine, alcohol, opiate and nicotine addiction;
(3) emotional disorders, e.g. depressive disorders, in particular “major depression”, manic-depressive disorders, organically-induced depressions, e.g. in connection with neurodegenerative illnesses such as Parkinson's or Alzheimer's disease;
(4) motor disturbances, including tremors, rigor, dyskinesias, dystonias, such as with Parkinson's disease, parkinsonian syndrome, (idiopathically, e.g. in Parkinson-plus-syndrome, or medication-induced, e.g. following L-dopa or neuroleptic treatment), Segawa syndrome, Tourette's syndrome, restless leg syndrome;
(5) sleeping disorders, including dopamine agonist triggered narcolepsy or sleeping disorders associated with Parkinson's disease;
(6) nausea: here dopamine antagonists can be used either alone or in combination with 5-HT3 antagonists;
(7) cognitive disorders and dementias;
(8) hyperprolactinemia; hyperprolactinomia and medically supported ablactation following pregnancy;
(9) glaucoma;
(10) attention deficit hyperactive syndrome (ADHS);
(11) autism, or disorders associated with autism, in particular in the case of compounds with strong serotoninergic active components;
(12) stroke, in particular in the case of compounds with strong serotoninergic active components.

A further therapeutic application that can be mentioned is the treatment and prevention of neurodegenerative diseases, since due to their neuroprotective effect the substances can delay or stop the destruction or loss of neurones as the cause or result of a pathophysiological episode. Such illnesses are for example amyotrophic lateral sclerosis, Alzheimer's disease, Huntington's chorea, epilepsy, Parkinson's disease or synucleopathias, e.g. of the Parkinson-plus-syndrome type.

Apart from the treatment of illnesses which clearly occur and/or continue with the involvement of the central nervous system, the substances according to the invention can also be used to treat other illnesses which are not associated with, not clearly associated with, or not exclusively associated with the central nervous system. Such illnesses are in particular forms of pain or disorders of the urinary tract, such as sexual dysfunction, in particular male erectile dysfunction and urinary incontinence. For the treatment of urinary incontinence compounds with strong serotoninergic active components are particularly suitable.

Subject-matter of the invention is therefore the use of a compound according to the invention for the production of a pharmaceutical preparation for the treatment of pains or of disorders of the urinary tract, in particular of male erectile dysfunction and urinary incontinence.

Illnesses for which the compounds according to the invention are particularly suitable are schizophrenia, depressive disorders, L-dopa- or neuroleptic drug-induced motor disturbances, Parkinson's disease, Segawa syndrome, restless leg syndrome, hyperprolactinemia, hyperprolactinomia, attention deficit hyperactive syndrome (ADHS) and urinary incontinence.

Motor disturbances which are particularly open to therapy with the substances according to the invention are in particular
motor disturbances associated with Parkinson's disease, e.g. rigor, tremor, dystonia and dyskinesia,
Segawa syndrome
neuroleptic drug-induced (delayed) extrapyramidal motor disturbances, in particular dyskinesia, dystonia and akathisia,
L-dopa-induced extrapyramidal motor disturbances, in particular dyskinesias and dystonias,
restless leg syndrome.

Finally, the pharmaceutical preparations according to the invention, depending on the illness to be treated, can also be in the form of a combined preparation for simultaneous or sequential administration.

For example, a sales unit, containing an L-dopa medication for treatment of Parkinson's disease, can also comprise a pharmaceutical composition containing one or more of the compounds according to the invention with, for example, a highly selective, partial agonist dopaminergic and/or serotoninergic profile of action. Here L-dopa and the compound according to the invention can be present in the same pharmaceutical formulation, e.g. a combined tablet, or also in different application units, e.g. in the form of two separate tablets. The two active substances can be administered simultaneously or separately as necessary.

In a combined preparation a sequential administration can, for example, be achieved by the form of administration, e.g. an oral tablet, having two different layers with differing release profiles for the various pharmaceutically active components. It will be clear to the person skilled in the art that in the context of the present invention various forms of administration and application administration schemes are conceivable which are all the subject-matter of the invention.

One embodiment of the invention therefore concerns a pharmaceutical preparation containing L-dopa or a neuroleptic drug and a compound according to the invention for simultaneous or timed sequential administration to the patient.

In another embodiment of the invention the sales unit can be a combined preparation or contain two application units, which contain two of the compounds according to the invention with different receptor profiles, e.g. a high affinity, highly selective D3-modulator and a high affinity 5-HT1a-modulator.

Also forming the subject-matter of the invention is a method for treatment of an illness selected from among the illnesses listed in more detail above, through the administration of one or more of the compounds according to the invention, in each case either alone or in combination with other pharmaceutical preparations to a mammal, in need of such treatment, wherein the term “mammal” also and in particular includes humans.

Normally the pharmaceutical preparations according to the invention comprise a pharmaceutical composition which apart from the compounds according to the invention, as described above, contain at least one pharmaceutically acceptable carrier or adjuvant.

It will be clear to the person skilled in the art that the pharmaceutical formulation can be designed differently depending on the envisaged administration route. Thus the pharmaceutical formulation can, for example, be adapted for intravenous, intramuscular, intracutaneous, subcutaneous, oral, buccal, sublingual, nasal, transdermal, inhalative, rectal or intraperitoneal administration.

Appropriate formulations and suitable pharmaceutical carriers or adjuvants, such as fillers, disintegrants, binding agents, lubricants, stabilisers, aromatics, antioxidants, preservatives, dispersion or dissolution agents, buffers or electrolytes, will be known to the person skilled in the art in the area of pharmaceuticals and are for example described in the standard works such as Sucker, Fuchs and Speiser (“Pharmazeutische Technologie” (Pharmaceutical Engineering), Deutscher Apotheker Verlag, 1991) and Remington (“The Science and Practice of Pharmacy”, Lippincott, Williams & Wilkins, 2000).

In a preferred embodiment of the invention the pharmaceutical compositions, containing the compounds according to the invention, are administered orally and can, for example, be in the form of capsules, tablets, powders, granulates, coated pills or a liquid.

Here the formulation can be designed as a rapid release form of administration, if fast taking effect is desired. Appropriate oral formulations are, for example, described in EP 0 548 356 or EP 1 126 821.

If, on the other hand, a delayed release is desired, a formulation with delayed active substance release offers itself. Appropriate oral formulations are also known from the prior art.

Alternative pharmaceutical preparations can, for example, be infusion or injection solutions, oils, suppositories, aerosols, sprays, plasters, microcapsules or microparticles.

The compounds of formulae I to VII are produced using methods that are in part already described in the literature (Bettinetti, L. et al. J. Med. Chem. 2002, 45, 4594-4597). In addition acid derivatives of type (A), which are either obtained commercially, synthesised according to the instructions in the literature or whose production methods are worked out in our laboratories, in the form of their carboxylic acid chlorides or alternatively through the use of the carboxylic acids by using special activation reagents such as hydroxybenzotriazole, hydroxyazabenzotriazole, HATU (Kienhöfer, A. Synlett 2001, 1811-1812) or TBTU (Knorr, R. Tetrahedron Lett. 1989, 30, 1927-1930) are activated and with the free base of type (C) converted to the derivatives of formulae I and VII:

Production of the compounds according to the invention takes place by conversion of an acid derivative A

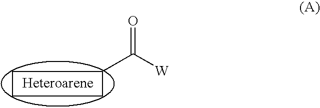

with a free base of general formula C

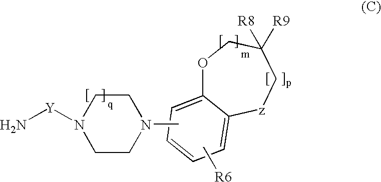

wherein:

W is selected from OH, Cl, Br or a group

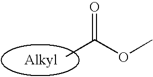

Heteroarene stands for a group of general formula Ia

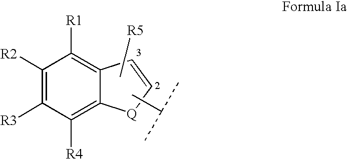

wherein R, R1, R2, R3, R4, R5, R6, R8, R9, Q, Z, m, p and q have the significance as defined in more detail above and wherein the crossed-through bonding in the heteroarene stands for the bonding of the —C(O)—W group to the 2- or 3-position of said heteroarene with formula Ia;

and wherein in the event that the substituent W is a hydroxy group, the appropriate acid group prior to the conversion with the free base of general formula C is activated by addition of activation reagents such as hydroxybenzotriazole, hydroxyazabenzotriazole, HATU or TBTU.

W is preferably chlorine, bromine or OH and particularly preferably chlorine or OH.

### SYNTHESIS OF EMBODIMENTS

Access to the commercially obtained heteroarene carboxylic acids (referred to here as “type A1”):

### Benzo[b]thiophene-2-carboxylic acid; 5-bromobenzo[b]thiophene-2-carboxylic acid; benzo[b]thiophene-3-carboxylic acid; benzofuran-2-carboxylic acid; indole-2-carboxylic acid; indole-3-carboxylic acid; 3-chlorobenzo[b]thiophene-2-carboxylic acid chloride

Heteroarene carboxylic acids of type A1 are commercially available, e.g. benzo[b]thiophene-2-carboxylic acid (e.g. from Aldrich, Taufkirchen; No.: 46,746-4); 5-bromobenzo[b]thiophene-2-carboxylic acid (e.g. from Maybridge, Tintangel, UK; No.: CC 31201); benzo[b]thiophene-3-carboxylic acid (e.g. from Maybridge, Tintangel, UK; No.: CC 12301); benzofuran-2-carboxylic acid (e.g. from Aldrich, Taufkirchen; No.: 30,727-0); indole-2-carboxylic acid (e.g. from Aldrich, Taufkirchen; No.: I-510-9) or indole-3-carboxylic acid (e.g. from Aldrich, Taufkirchen; No: 28,473-4). Derivates of the carboxylic acids, such as 3-chlorobenzo[b]thiophene-2-carboxylic acid chloride (e.g. from Maybridge, Tintangel, UK; No.: BTB 00300), are likewise commercially available.

De novo synthesis of heteroarene carboxylic acids (referred to here as “type A2”):

### Benzofuran-3-carboxylic acid; 6-cyanoindole-2-carboxylic acid; 5-cyanobenzo[b]thiophene-2-carboxylic acid; 6-ethynylbenzo[b]thiophene-2-carboxylic acid

The heteroarene carboxylic acids of type A2 were produced in our laboratory as described in the following.

### Benzofuran-3-carboxylic acid

Benzofuran-2,3-dicarboxylic acid (2.06 g; 10.0 mmol) (Aldrich, Taufkirchen; No.: 64,274-6) is agitated with copper powder (1.15 g; 18.1 mmol) and chinoline (2.0 g; 15.48 mmol) together for 2 hours at 195° C. in the oil bath. Following cooling it is taken up in dichloromethane, drawn through a fritted glass filter with suction, and the residue washed with dichloromethane. The filtrate is evaporated in the rotary evaporator and the residue purified by flash chromatography (CH₂Cl₂-MeOH: 95-5 with 5% HCOOH) and then (CH₂Cl₂-MeOH: 98-2 with 5% HCOOH)

Yield: 845 mg (52%)

MS m/z 162 (M⁺). ¹HNMR (CDCl₃, 360 MHz) ∂ (ppm): 7.38-7.45 (m, 2H, H-5, H-6), 7.55-7.61 (m, 1H, H-7), 8.10-8.16 (m, 1H, H-4), 8.39 (s, 1H, H-2).

### 6-cyanoindole-2-carboxylic acid

The 6-cyanoindole-2-carboxylic acid methyl ester (0.05 g (0.24 mmol)) produced according to the literature (Dann, O.; Wolff, H. P.; Schlee, R.; Ruff, J. Liebigs Annalen der Chemie (“J. Liebig's History of Chemistry”), 1986, 2164-2178) is dissolved in 5 ml methanol. Then 2.5 ml 2N NaOH are added and agitation is performed for 16 hours at ambient temperature. The reaction solution is concentrated in the rotary evaporator and diluted with water, then washed with hexane, adjusted with HCl to pH 3-4 and taken up in diethyl ether. Following drying with MgSO₄ the solvent is evaporated.

Yield: 0.04 g (87%).

MS: m/z 187 ((M+H)⁺).

### 5-cyanobenzo[b]thiophene-2-carboxylic acid

The 5-cyanobenzo[b]thiophene-2-carboxylic acid methyl ester (0.08 g, 0.36 mmol) produced according to the literature (Bridges, A. J.; Lee, A.; Maduakor, E. C.; Schwartz, C. E. Tetrahedron Letters, 1992, 33, 7499-7502) is dissolved in 8 ml THF and cooled to 0° C. Then methanol (8 ml) and 2N NaOH (4 ml) are added dropwise and agitation is performed for 4 hours at ambient temperature. Then dilution takes place with water and THF, the organic solvent is rotated off and the aqueous phase washed with ethyl acetate. The aqueous phase is adjusted with HCl to pH 2 and extracted several times with diethyl ether. The combined ether phases are dried with magnesium sulphate and evaporated with the rotary evaporator.

Yield: 0.06 g (80%) white solid matter

m/z 203 (M⁺). IR (NaCl) v (cm⁻¹): 3375, 2359, 2226 (CN), 1676 (COOH), 1598, 1385, 1086, 720. ¹H-NMR (CDCl₃, 360 MHz) δ (ppm): 3.68 (br.s, 1H, COOH), 7.86 (dd, J=1.8 Hz, J=8.5 Hz, 1H, H-6), 8.19 (s, 1H, H-3), 8.30 (d, J=8.2 Hz, 1H, H-7), 8.55 (d, J=1.4 Hz, H-4). ¹³C-NMR (CD₃OD, 90 MHz) δ (ppm): 109.9 (C-5), 119.8 (CN), 125.3 (C-7), 129.4 (C-4), 130.9 (C-6), 131.4 (C-3), 134.1 (C-3a), 140.0 (C-2), 147.4 (C-7a).

### 6-ethynylbenzo[b]thiophene-2-carboxylic acid

The 6-iodobenzo[b]thiophene-2-carboxylic acid methyl ester (0.15 g, 0.47 mmol) produced according to the literature (Bridges, A. J.; Lee, A.; Maduakor, E. C.; Schwartz, C. E. Tetrahedron Letters, 1992, 33, 7499-7502) is dissolved in dry THF (5 ml) under N₂-atmosphere, then finely divided CuI (3.6 mg, 0.019 mmol, 4 mol %) and PdCl₂(PPh₃)₂ (6.6 mg, 0.01 mmol, 2 mol %) are added and under agitation NEt₃ (0.10 ml 0.70 mmol) and then trimethylsilylacetylene (0.10 ml, 0.70 mmol) dissolved in 2 ml THF are added dropwise. Following agitation at ambient temperature for 18 hours the solvent is drawn off using the rotary evaporator and the residue purified by flash chromatography (hexane-ethyl acetate: 99-1).

Yield: 0.11 g (81%) white solid matter

M.P.: 110° C. MS m/z 288 (M⁺). IR (NaCl) v (cm⁻¹): 3406 (CCH), 2955, 2898,2154 (CC), 1716 (C═O), 1250, 1250′, 756. ¹H-NMR (CDCl₃, 360 MHz) δ (ppm): 0.27 (s, 9H, Si(CH₃)₃), 3.94 (s, 3H, OCH₃), 7.46 (dd, J=1.4 Hz, J=8.3 Hz, 1H, H-5), 7.78 (dd, J=0.5, J=8.3 Hz, 1H, H-4), 7.97-7.98 (m, 1H, H-3), 8.01 (d, J=0.9 Hz, 1H, H-7). ¹³C-NMR (CDCl₃, 90 MHz) δ (ppm): 0.0, 0.1, 0.2, 52.6, 96.2, 104.6, 121.9, 125.2, 126.6, 128.5, 130.3, 134.7, 138.4, 141.9, 162.9.

The 6-trimethylsilylethynylbenzo[b]thiophene-2-carboxylic acid methyl ester produced in this way (23.0 mg, 0.08 mmol) is dissolved in THF (2 ml), cooled to −15° C. and 1M NH₄Bu₄F-solution (in THF) (0.09 ml, 0.09 mmol) added dropwise under agitation. After 30 minutes the reaction mixture is concentrated in the rotary evaporator at ambient temperature and extracted on silica gel. Purification with flash chromatography (hexane-ethyl acetate: 99-1) produces 6-ethynylbenzo[b]thiophene-2-carboxylic acid methyl ester.

Yield: 0.11 g (79%) white solid matter

M.P.: 147° C. MS m/z 288 (M⁺). IR (NaCl) v (cm⁻¹): 3245 (CCH), 2921 (CH₃), 2154 (CH₃), 1699 (COOCH₃), 1069, 756. ¹H-NMR (CDCl₃, 360 MHz) δ (ppm): 3.19 (s, 1H, CCH), 3.95 (s, 3H, OCH₃), 7.49 (dd, J=1.4 Hz, J=8.4 Hz, 1H, H-5), 7.81 (dd, J=0.5 Hz, J=8.4 Hz, H-4), 8.00 (s, 1H, H-3), 8.02 (d, J=0.7 Hz, 1H, H-7). ¹³C-NMR (CDCl₃, 90 MHz) δ (ppm): 52.6 (OCH₃), 78.7, 83.3, 120.8, 125.3, 126.2, 128.5, 130.2, 134.9, 138.7, 141.9, 162.9.

6-ethynylbenzo[b]thiophene-2-carboxylic acid methyl ester (0.08 g, 0.36 mmol) is dissolved in 8 ml THF and cooled to 0° C. Then methanol (8 ml) and 2N NaOH (4 ml) are added dropwise and agitation is performed for 4 hours at ambient temperature. Then water and THF are added, the organic solvent is evaporated and the remaining, aqueous phase is washed with ethyl acetate. The aqueous phase is adjusted with HCl to pH 2 and extracted several times with diethyl ether. The combined ether phases are dried with magnesium sulphate and evaporated with the rotary evaporator.

Yield: 0.08 g (94%) white solid matter

M.P.: 214° C. MS m/z 202 (M⁺). IR (NaCl) v (cm⁻¹): 3288, 2952, 2816, 2103, 1672, 1516, 1421, 1182, 1045, 813, 758. ¹H-NMR (CD₃OD, 360 MHz) δ (ppm): 3.65 (s, 1H, CCH), 7.51 (dd, J=1.2 Hz, 8.2 Hz, 1H, H-5), 7.92 (d, J=8.2 Hz, 1H, H-4), 8.06 (s, 1H, H-3), 8.07 (s, 1H, H-7). ¹³C-NMR (CDCl₃, 90 MHz) δ (ppm): 80.1, 84.2, 122.3, 126.5), 127.4, 129.5, 131.1, 137.8, 140.3, 143.4), 165.6.

Production of the amines:

The amine components of the compounds according to the invention were produced as described in the following, wherein the grouping of the amines in types “C1” to “C9” took place according to chemical structural characteristics.

Production of type C1 amines:

### 4-(4-(2,3-dihydrobenzofuran-7-yl)piperazin-1-yl)butylamine; 4-(4-(chroman-8-yl)piperazin-1-yl)butylamine

The synthesis of the piperazine-substituted dihydrobenzofuran takes place analogously to the literature (Kerrigan, F. Tetrahedron Lett. 1998, 2219-2222) until 2,3-dihydrobenzofuran-7-yl)piperazine has been obtained with a yield of 54% over 4 reaction steps.

Then the free base is alkylated with a cyanoalkyl halogenide of appropriate chain length as illustrated by way of example in the following reaction diagram:

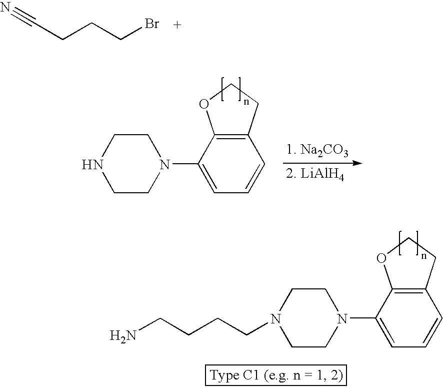

For this 3.7 mmol of appropriately substituted piperazine and 0.8 g (7.5 mmol) Na₂CO₃ are dissolved in 20 ml acetonitrile, 3,1 mmol ω-bromoalkylnitrile are added and heated for 15 hours with recycling, then cooled to ambient temperature and the solution evaporated in the vacuum. The residue is absorbed in water and the aqueous phase extracted with methylene chloride, this is dried (with MgSO₄) and the solvent is evaporated. Purification by flash chromatography (e.g. with CHCl₃-EtOAc:1-1) produces the corresponding ω-(4-phenylpiperazin-1yl)alkylnitrile.

Then 0.5 mmol ω-(4-phenylpiperazin-1yl)alkylnitrile are dissolved in 5 ml dry diethyl ether and cooled to 0° C. Then 1.0 ml LiAlH₄ solution (1 M in diethyl ether) are slowly added dropwise and agitated for 1 hour at ambient temperature. Following cooling again to 0° C. saturated NaHCO₃ solution is added, filtration is performed through a fritted glass filter with Celite/MgSO₄/Celite and washing is performed with methylene chloride. Evaporation of the filtrate results in the desired ω-(4-phenylpiperazin-1yl)alkylamine.

### 4-(4-(2,3-dihydrobenzofuran-7-yl)piperazin-1-yl)butylamine

The synthesis of 4-(4-(2,3-dihydrobenzofuran-7-yl)piperazin-1-yl)butylamine takes place in the manner described above.

Yield: 0.27 g (86% over 2 reaction steps).

MS: m/z 275 (M⁺). IR: (NaCl): 3359, 2939, 2820, 1609, 1487, 1456, 1254, 1190, 1132, 1012, 942, 870, 755, 661. ¹H NMR (CDCl₃, 360 MHz) δ (ppm): 1.43-1.63 (m, 4H,CH₂-CH₂); 2.34-2.40 (m, 2H, H₂N—CH₂); 2.62 (m, 4H, pip); 2.72-2.74 (m, 2H, O—CH₂—CH₂); 3.15-3.21 (m, 6H, pip, CH₂N); 4.56-4.61 (m, 2H, O—CH₂—CH₂); 6.69-6.71 (m, 1H, phenyl); 6.77-6.86 (m, 2H, phenyl).

### 4-(4-(chroman-8-yl)piperazin-1-yl)butylamine

The production of 4-(4-(chroman-8-yl)piperazin-1-yl)butylamine takes place analogously to the conditions described for 4-(4-(2,3-dihydrobenzofuran-7-yl)piperazin-1yl)butylamine.

Yield: 0.058 g (57% over 2 reaction steps).

MS: m/z 289 (M⁺). IR: (NaCl): 3354, 2933, 2870, 2814, 1664, 1479, 1461, 1247, 1196, 1024, 870,737. ¹H NMR (CDCl₃, 360 MHz) δ (ppm): 1.46-1.59 (m, 4H,CH₂—CH₂); 1.96-2.03 (m, 2H, O—CH₂—CH₂—CH₂); 2.39-2.44 (m, 2H, CH₂—N); 2.65 (m, 4H, pip); 2.70-2.74 (m, 2H, O—CH₂—CH₂—CH₂); 2.77-2.80 (m, 2H, CH₂—NH₂); 3.08 (m, 4H, pip); 4.24-4.27 (m, 2H, O—CH₂—CH₂—CH₂); 6.71-6.79 (m, 3H, phenyl).

Production of type C2 amines:

### 4-(4-(2,3,4,5-tetrahydrobenzo[b]oxepin-9-yl)piperazin-1-yl)butylamine

By extension to the synthesis instructions according to type C1, 5-, 6- and 7-ring annulated bicyclical systems can be produced according to the following reaction diagram:

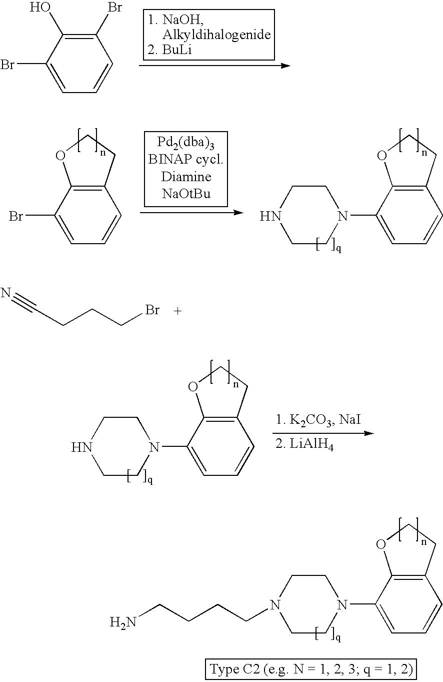

As an example for the diagram shown above the synthesis of 4-(4-(2,3,4,5-tetrahydrobenzo[b]oxepin-9-yl)piperazin-1-yl)butylamine is described: For this to begin with 2,6-dibromophenol (28.8 mmol) is heated for 17 hours under basic conditions (aqueous NaOH) with 1,4-dibromobutane (28.8 mmol) with reflux. The resulting 2,6-dibromophenoxybutylbromide (16.75 mmol) is dissolved in THF/hexane (4/1), cooled to −80° C. and a 2.5 M solution of butyllithium in hexane (17.1 mmol) is slowly added dropwise. The annulated 9-bromo-2,3,4,5-tetrahydrobenzo[b]oxepine (4 mmol) obtained in this way is suspended with NaOtBu (20 mmol), Pd₂(dba)₃ (2 mol %), BINAP (2 mol %) and piperazine (8 mmol) in 5 ml dry toluene and heated for 6 hours at 117° C. Following work up, the 1-(2,3,4,5-tetrahydrobenzo[b]oxepin-9-yl)piperazine (1,5 mmol) obtained and 0.63 g (4.5 mmol) K₂CO₃ are dissolved in 20 ml acetonitrile, 0.15 ml (1.5 mmol) 4-bromobutyronitrile are added and heating performed for 15 hours with reflux, then cooling to ambient temperature takes place and the solution is evaporated in the vacuum. The residue is taken up in water and the aqueous phase extracted with methylene chloride, this is dried (with MgSO₄) and the solvent is evaporated. Purification with flash chromatography (CHCl₃-EtOAc:1-1) produces the corresponding 4-(4-(2,3,4,5-tetrahydrobenzo[b]oxepin-9-yl)piperazin-1yl)butyronitrile. Of this 0.5 mmol are then dissolved in 5 ml dry diethyl ether and cooled to 0° C. Then 1.0 ml LiAlH₄ solution (1 M in diethyl ether) are slowly added dropwise and agitated for 1 hour at ambient temperature. Following cooling again to 0° C. saturated NaHCO₃ solution is added, filtration is performed through a fritted glass filter with Celite/MgSO₄/Celite and washing is performed with methylene chloride. Evaporation of the filtrate produces 4-(4-(2,3,4,5-tetrahydro-benzo[b]oxepin-9-yl)piperazin-1-yl)butylamine.

Yield: 0.52 g (86%).

(APCl) MS: m/z 304 ((M+H)⁺). IR: (NaCl): 2933, 2870, 2814, 1666, 1579, 1475, 1450; 1246, 1192, 1038, 785, 733. ¹H NMR (CDCl₃, 360 MHz) δ (ppm): 1.47-1.63 (m, 4H, CH₂—CH₂); 1.68-1.75 (m, 2H, O—CH₂—CH₂—CH₂—CH₂); 1.93-2.00 (m, 4H, H₂O, O—CH₂—CH₂—CH₂—CH₂); 2.41-2.45 (m, 2H, CH₂—N); 2.61-2.65 (m, 4H, pip); 2.73-2.81 (m, 4H, O—CH₂—CH₂—CH₂—CH₂, CH₂—NH₂); 3.10-3.12 (m, 4H, pip); 3.98-4.00 (m, 2H, O—CH₂—CH₂—CH₂—CH₂); 6.77-6.81 (m, 2H, Phenyl); 6.88-6.93 (m, 1H, Phenyl). ¹³C NMR (CDCl₃, 90 MHz) δ (ppm): 153.5; 144.8; 136.9; 123.9; 123.4; 116.8; 73.3; 58.6; 53.7; 51.0; 42.0; 34.5; 32.5; 31.6; 26.1; 24.3.

Production of type C3 amines:

### 4-(4-(2,3-dihydrobenzofuran-7-yl)-1,4-diazepan-1-yl)butylamine; 4-(4-(chroman-8-yl)-1,4-diazepan-1-yl)butylamine; 4-(4-(2,3,4,5-tetrahydrobenzo[b]oxepin-9-yl)-1,4-diazepan-1-yl)butylamine

In place of the piperazine for the Pd-catalysed substitution described under type C2 other cyclical diamines, such as 1,4-diazepane, can be used for the production of the amine components.

Thus the synthesis of 4-(4-(2,3-dihydrobenzofuran-7-yl)-1,4-diazepan-1-yl)-butylamine takes place analogously to the production of the type C2 amines.

Yield: 0.28 g (96%)

(APCl) MS: m/z 290 ((M+H)⁺). ¹H NMR (CDCl₃, 360 MHz) δ (ppm): 1.44-1.49 (m, 2H, CH₂—CH₂); 1.52-1.57 (m, 2H, CH₂—CH₂); 1.79-1.85 (m, 2H, N(CH₂—CH₂)N(CH₂—CH₂—CH₂)N; 1.95-1.99 (m, 2H, N(CH₂—CH₂)N(CH₂—CH₂—CH₂)N); 2.50-2.53 (m, 2H, CH₂—N); 2.70-2.73 (m, 4H, N(CH₂—CH₂)N(CH₂—CH₂—CH₂)N); 2.80-2.81 (m, 2H, CH₂—NH₂); 3.18 (t, J=8.9 Hz, 2H, O—CH₂—CH₂); 3.43-3.55 (m, 2H, N(CH₂—CH₂)N(CH₂—CH₂—CH₂)N); 3.51-3.53 (m, 2H, N(CH₂—CH₂)N(CH₂—CH₂—CH₂)N); 4.53 (t, J=8.9 Hz, 2H, O—CH₂—CH₂); 6.61-6.62 (m, 1H, H-phenyl); 6.70-6.71 (m, 1H, H-phenyl); 6.75-6.78 (m, 1H, H-phenyl).

The synthesis of 4-(4-(chroman-8-yl)-1,4-diazepan-1-yl)-butylamine takes place by analogy to the manner described for type C2.

Yield: 0.27 g (88%) MS: m/z 303 (M⁺). ¹H NMR (CDCl₃, 360 MHz) δ (ppm): 1.42-1.49 (m, 4H, CH₂—CH₂); 1.62-1.70 (m, 2H, N(CH₂—CH₂)N(CH₂—CH₂—CH₂)N); 1.92-2.03 (m, 2H, O—CH₂—CH₂—CH₂); 2.50-2.54 (m, 2H, CH₂—N); 2.70-2.84 (m, 8H, N(CH₂—CH₂)N(CH₂—CH₂—CH₂)N); 3.28-3.32 (m, 2H, CH₂—NH₂); 4.20-4.23 (m, 2H, O—CH₂—CH₂); 6.61-6.63 (m, 1H, H-phenyl); 6.73-6.75 (m, 2H, H-phenyl).

### 4-(4-(2,3,4,5-tetrahydrobenzo[b]oxepin-9-yl)-1,4-diazepan-1-yl)butylamine is synthesised analogously.

Yield: 0.3 g (94%)

(APCl) MS: m/z 318 ((M+H⁺)).

Production of type C4 amines:

### 4-(4-(chroman-7-yl)piperazin-1-yl)butylamine

Production of the type C4 amines takes place by analogy to the synthesis of the type C2 amines, wherein for the synthesis of 4-(4-(chroman-7-yl)piperazin-1-yl)butylamine 2,5-dibromophenol (Interchim Building Blocks, Montlucon, France; No.: BC708) is converted with 1,3-dibromopropane.

Yield: 0.14 g (92%).

(APCI) MS: m/z 304 (M⁺).

Production of type C5 amines:

### 4-(4-(2,3-dihydrobenzofuran-5-yl)piperazin-1-yl)butylamine; 4-(4-(chroman-6-yl)piperazin-1-yl)butylamine; 4-(4-(2,3,4,5-tetrahydrobenzo[b]oxepin-7-yl)piperazin-1-yl)butylamine

Production of the type C4 amines takes place by analogy to the synthesis of the type C2 amines, wherein for the synthesis of the bicylically annulated bromoheteroarene system the 2,4-dibromophenol obtainable commercially from Aldrich, Taufkirchen (No.: 25,816-4) is converted with 1,ω-dibromalkanes.

### 4-(4-(2,3-dihydrobenzofuran-5-yl)piperazin-1-yl)butylamine

For the production of 4-(4-(2,3-dihydrobenzofuran-5-yl)piperazin-1-yl)butylamine 2,4-dibromophenol is converted with 1,2-dibromomethane.

Yield: 0.28 g (92%).

(APCl) MS: m/z 276 (M⁺). ¹H NMR (CDCl₃, 360 MHz) δ (ppm): 1.46-1.61 (m, 4H, CH₂—CH₂); 2.40 (t, J=7.5 Hz, 2H, H₂N—CH₂); 2.59-2.62 (m, 4H, pip); 2.72 (t, J=7.0 Hz, 2H, CH₂N); 3.06-3.09 (m, 4H, pip); 3.16 (t, J=8.6 Hz, 2H, O—CH₂—CH₂); 4.51 (t, J=8.6 Hz, 2H, O—CH₂—CH₂); 6.69 (d, J=8.4 Hz, 1H, phenyl); 6.72 (dd, J=8.4 Hz, J=2.2 Hz, 1H, H-phenyl); 6.86 (d, J=2.2 Hz, 1H, H-phenyl).

### 4-(4-(chroman-6-yl)piperazin-1-yl)butylamine

For the synthesis of 4-(4-(chroman-6-yl)piperazin-1-yl)butylamine 1,3-dibromopropane is used.

Yield: 0.2 g (97%).

(APCl) MS: m/z 290 ((M+H)⁺).

### 4-(4-(2,3,4,5-tetrahydrobenzo[b]oxepin-7-yl)piperazin-1-yl)butylamine

The synthesis of 4-(4-(2,3,4,5-tetrahydrobenzo[b]oxepin-7-yl)piperazin-1-yl)butylamine takes place by conversion of the 2,4-dibromophenol with 1,4-dibromobutane.

Yield: 0.549 g (90%).

(APCI) MS: m/z 304 ((M+H)⁺).

Production of type C6 amines:

### 4-((4-benzo[1,3]dioxol-4-yl)piperazin-1-yl)butylamine

Type C6 amines are synthesised analogously to the production instructions for type C2, wherein for the synthesis of 4-((4-benzo[1,3]dioxol-4-yl)piperazin-1-yl)butylamine the 4-bromo-1,3-benzodioxol purchased from Maybridge, Tintangel, UK (No: CC01710) was used for the Pd-coupled amine substitution. The subsequent alkylation and reduction produces 4-((4-benzo[1,3]dioxol-4-yl)piperazin-1yl)butylamine.

Yield: 0.53 g (96%).

(APCl) MS: m/z 278 ((M+H)⁺).

Production of type C7 amines:

### 4-((4-benzo[1,3]dioxol-5-yl)piperazin-1yl)butylamine; 4-(4-(2,3-dihydrobenzo[1,4]dioxin-6-yl)piperazin-1-yl)butylamine; 4-(4-(3,4-dihydro-2H-benzo[b][1,4]dioxepin-7-yl)piperazin-1-yl)butylamine

Type C7 amines are produced by analogy to the synthesis of the type C2 amines through the use of appropriately substituted bicyclically annulated bromoarenes.

### 4-((4-benzo[1,3]dioxol-5-yl)piperazin-1yl)butylamine

Conversion of 5-boromo-1,3-benzodioxol (Aldrich, Taufkirchen; No.: 28,831-4) led to the synthesis of 4-(4-benzo[1,3]dioxol-5-yl)piperazin-1yl-butylamine.

Yield: 0.24 g (96%)

(APCI) MS: m/z 278 ((M+H)⁺). ¹H NMR (CDCl₃, 360 MHz) δ (ppm): 1.44-1.61 (m, 4H, CH₂—CH₂); 1.64-1.69 (brs, 2H NH₂); 2.40 (t, J=7.0 Hz, 2H, H₂N—CH₂); 2.58-2.61 (m, 4H, pip); 2.73 (t, J=7.0 Hz, 2H, CH₂N); 3.06-3.09 (m, 4H, pip); 5.88 (s, 2H, O—CH₂—O); 6.36 (d, J=8.4, Hz, J=2.4 Hz, 1H, H-phenyl); 6.56 (d, J=2.4 Hz, 1H, H-phenyl); 6.71 (d, J=8.4 Hz, 1H, H-phenyl).

### 4-(4-(2,3-dihydrobenzo[1,4]dioxin-6-yl)piperazin-1-yl)butylamine

The use of 6-bromo-2,3-dihydrobenzo[1,4]dioxin (Lancester, Frankfurt; No.: 6207) led to the production of 4-(4-(2,3-dihydrobenzo[1,4]dioxin-6-yl)piperazin-1-yl)butylamine.

Yield: 0.12 g (59%).

(APCl) MS: m/z 292 ((M+H)⁺). IR: (NaCl): 2937, 2875, 2817, 1587, 1508, 1454, 1284, 1219, 1070, 752. ¹H NMR (CDCl₃, 360 MHz) δ (ppm): 1.46-1.67 (m, 4H, CH₂—CH₂); 1.88-1.98 (brs, 2H, NH₂); 2.40 (t, J=7.0 Hz, 2H, CH₂—N); 2.57-2.61 (m, 4H, pip); 2.74 (t, J=7.0 Hz, 2H, CH₂—NH₂); 3.06-3.11 (m, 4H, pip); 4.18-4.25 (m, 4H, O—CH₂—CH₂—O); 6.44-6.48 (m, 2H, H-phenyl); 6.75-6.78 (m, 1H, H-phenyl).

### 4-(4-(3,4-dihydro-2H-benzo[b][1,4]dioxepin-7-yl)piperazin-1-yl)butylamine

The production of 4-(4-(3,4-dihydro-2H-benzo[b][1,4]dioxepin-7-yl)piperazin-1-yl)butylamine takes place on the basis of 7-bromo-3,4-dihydro-2H-1,5-benzodioxepine purchased from Maybridge, Tintangel, UK (No.: CC 13210) analogously to the conditions described for the synthesis of the production of the type C2 amines.

Yield: 0.58 g (95%)

(APCl) MS: m/z 306 (M+H)⁺).

Production of type C8 amines:

### 4-(4-(2,3-dihydrobenzo[1,4]dioxin-6-yl)-1,4-diazepan-1-yl)butylamine

The synthesis of the type C8 amines takes place analogously to the instructions for the production of the type C3 amines.

For the production of 4-(4-(2,3-dihydrobenzo[1,4]dioxin-6-yl)-1,4-diazepan-1-yl)butylamine, 6-bromo-2,3-dihydrobenzo[1,4]dioxin (Lancester, Frankfurt; No.: 6207) was used.

Yield: 0.6 g (98%)

(APCl) MS: m/z 306 ((M+H)⁺). ¹H NMR (CDCl₃, 360 MHz) δ (ppm): 1.53-1.59 (m, 4H, CH₂—CH₂); 1.92-1.99 (m, 2H, O—CH₂—CH₂—CH₂—O); 2.48-2.52 (m, 2H, CH₂—N); 2.62-2.65 (m, 2H, N(CH₂—CH₂)N(CH₂—CH₂—CH₂)N); 2.73-2.79 (m, 4H, N(CH₂—CH₂)N(CH₂—CH₂—CH₂)N, CH₂—NH₂); 3.21-3.50 (m, 6H, N(CH₂—CH₂)N(CH₂—CH₂—CH₂)N); 4.16-4.19 (m, 2H, O—CH₂—CH₂—CH₂—O); 4.22-4.24 (m, 2H, O—CH₂—CH₂—CH₂—O); 6.18-6.21 (m, 2H, H-phenyl); 6.71-6.74 (m, 1H, H-phenyl).

Production of type C9 amines:

### 8-(4-(4-aminobutyl)piperazin-1-yl)-6-chloro-3,4-dihydro-2H-benzo[1,4]oxazin-3-one

Type C9 amines are synthesised according to the following reaction diagram on the basis of 2-amino-4-chloro-6-nitrophenol:

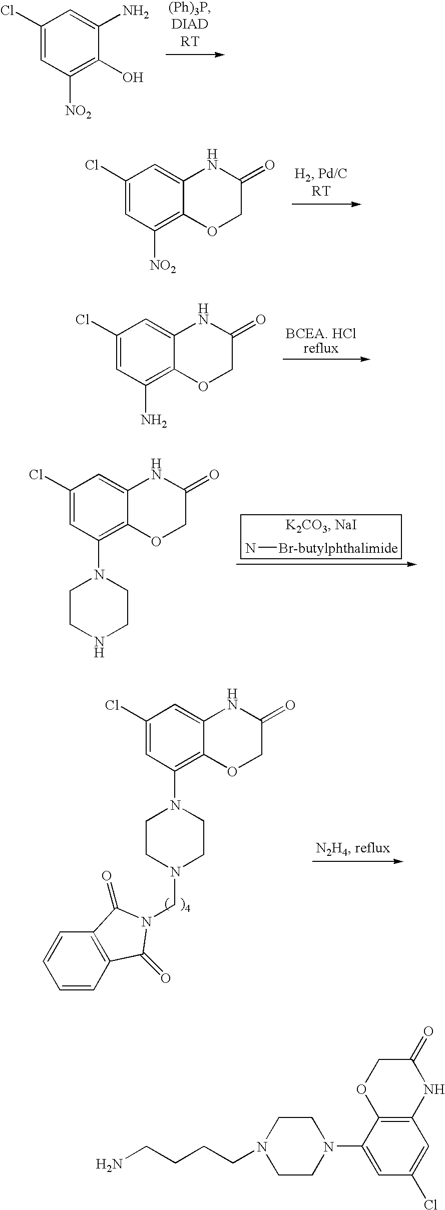

A solution of diisopropylazodicarboxylate (DIAD; 5.7 ml, 29.5 mmol) in dry THF (10 ml) is slowly added dropwise to a cooled solution (0° C.) of 5 g (26.5 mmol) 2-amino4-chloro-6-nitrophenol (Aldrich, Taufkirchen; No. 530387I), 2.4 g (26.5 mmol) methylglycolate and 7.77 g (29.2 mmol) triphenylphosphine in dry THF (200 ml). The reaction solution is agitated for 4 days at ambient temperature, and then the solvent is evaporated off in the vacuum and the resultant oil suspended in ethanol. In doing so 6-chloro-8-nitro-3,4-dihydro-2H-benzo[1,4]oxazin-3-one is precipitated as a dark green solid matter.

Yield: 3.6 g (59%).

IR (NaCl) v (cm⁻¹): 3390; 2923; 2854; 1707; 1631; 1473; 1342; 1028; 893. ¹H NMR (CDCl₃, 600 MHz) δ (ppm): 4.86 (s, 2H, O—CH₂—CONH); 7.65 (d, J=2.5 Hz, 1H, H-5); 8.02 (d, J=2.5 Hz, 1H, H-7).

0.8 g Pd/C are added to a solution of 2.5 g (10.9 mmol) 6-Chloro-8-nitro-3,4-dihydro-2H-benzo[1,4]oxazin-3-one in 50 ml EtOH/EtOAc (1/1; v/v) and then agitated for 24 hours under an H₂ atmosphere. The reaction mixture is filtered through Celite and the filtrate concentrated in the rotary evaporator. The residue is purified by flash chromatography (CH₂Cl₂-MeOH: 90-10) and provides 8-amino-6-chloro-3,4-dihydro-2H-benzo[1,4]oxazin-3-one.

Yield: 0.93 g (43%).

(APCl) MS: m/z 199 ((M+H)⁺). IR (NaCl) v (cm⁻¹): 3365; 2925; 2854; 1704; 1631; 1414; 771. ¹H NMR (CD₃OD, 600 MHz) δ (ppm): 4.51 (s, 2H, O—CH₂—CONH); 6.22 (d, J=2.5 Hz, 1H, H-7); 6.40 (d, J=2.5 Hz, 1H, H-5).

Bischloroethyleneamine hydrochloride (BCEA; 0.87 g, 4.9 mmol) are added to a solution of 0.93 g (4.7 mmol) 8-amino-6-chloro-3,4-dihydro-2H-benzo[1,4]oxazin-3-one in 50 ml chlorobenzol and heated for 80 hours with recycling. Concentration of the reaction mixture in the rotary evaporator and purification of the resultant residue by flash chromatography (CH₂Cl₂-MeOH-Et₃N: 80-18-2) provides 6-chloro-8-piperazin-1-yl-3,4-dihydro-2H-benzo[1,4]oxazin-3-one.

Yield: 0.50 g (45%).

(APCl) MS: m/z 268 ((M+H)⁺). IR (NaCl) v (cm⁻¹): 3392; 2848; 1689; 1620; 1591; 1396; 1228; 1036. ¹H NMR (CD₃OD, 600 MHz) δ (ppm): 2.96-2.98 (m, 4H, pip); 3.04-3.06 (m, 4H, pip). 4.57 (s, 2H, O—CH₂—CONH); 6.44 (d, J=2.9 Hz, 1H, H-5); 6.64 (d, J=2.9 Hz, 1H, H-7).

6-chloro-8-piperazin-1-yl-3,4-dihydro-2H-benzo[1,4]oxazin-3-one (0.5 g; 1.9 mmol), K₂CO₃ (0.8 g; 5.8 mmol) and NaI (0.6 g, 0.4 mmol) are dissolved in 10 ml dry acetonitrile. Then N-(4-bromobutyl)phthalimide (0.63 g; 2.2 mmol) is added and heated for 15 hours with reflux. Following cooling to ambient temperature the solvent is evaporated in the vacuum and the residue is purified by flash chromatography (CH₂Cl₂-MeOH: 95-5), which leads to 2-(4-(4-(4-chloro-3-oxo-3,4-dihydro-2H-benzo[1,4]oxazin-8-yl)piperazin-1-yl)butyl)isoindole-1,3-dione.

Yield: 0.2 g (22%).

(APCl) MS: m/z 469 ((M+H)⁺). IR (NaCl) v (cm⁻¹): 1768; 1707; 1620; 1518; 1398; 1223; 1047; 908. ¹H NMR (CDCl₃, 360 MHz) δ (ppm): 1.52-1.61 (m, 2H, CH₂—CH₂); 1.70-1.78 (m, 2H, CH₂—CH₂); 2.42 (t, J=7.5 Hz, 2H, CH₂—N); 2.54-2.58 (m, 4H, pip); 3.06-3.10 (m, 4H, pip). 3.72 (t, J=7.0 Hz, 2H, CH₂N(CO)₂); 4.64 (s, 2H, O—CH₂—CONH); 6.26 (d, J=2.7 Hz, 1H, H-5); 6.58 (d, J=2.7 Hz, 1H, H-7); 7.70-7.73 (m, 2H, isoindole); 7.83-7.86 (m, 2H, isoindole); 8.47 (brs, 1H, NHCO).

A solution of hydrazine hydrate 80% (0.2 ml; 5.5 mmol) in ethanol (5 ml) is carefully added dropwise to a suspension of 2-(4-(4-(6-chloro-3-oxo-3,4-dihydro-2H-benzo[1,4]oxazin-8-yl)piperazin-1-yl)butyl)isoindol-1,3-dione (0.2 g; 0.43 mmol) in 10 ml ethanol. Heating then takes place for 30 minutes with reflux followed by cooling to ambient temperature and evaporation of the solvent in the rotary evaporator. The residue is purified by flash chromatography (CH₂Cl₂-MeOH-Et₃N: 80-18-2) and produces 8-(4-(4-aminobutyl)piperazin-1-yl)-6-chloro-3,4-dihydro-2H-benzo[1,4]oxazin-3-one.

Yield: 0.12 g (80%).

(APCl) MS: m/z 339 ((M+H)⁺). IR (NaCl) v (cm⁻¹): 2821; 1699; 1653; 1475; 1296; 1230; 750. ¹H NMR (CD₃OD, 360 MHz) δ (ppm): 1.64-1.69 (m, 4H, CH₂—CH₂); 2.45-2.49 (m, 2H, CH₂N); 2.64-2.69 (m, 4H, pip); 2.87-2.91 (m, 2H, CH₂—NH₂); 3.12-3.15 (m, 4H, pip); 4.60 (s, 2H, O—CH₂—CONH); 6.48 (d, J=2.7 Hz, 1H, H-5); 6.66 (d, J=2.7 Hz, 1H, H-7).

### SYNTHESIS OF THE EXAMPLE COMPOUNDS

### EXAMPLE 1

### N-(4-(4-(2,3-dihydrobenzofuran-7-yl)piperazin-1-yl)butyl)benzo[b]thiophen-2-ylcarbamide

Benzo[b]thiophene-2-carboxylic acid (21 mg, 0.12 mmol) is dissolved in dry methylene chloride (4 ml) and DIEA (0.07 ml, 0.42 mmol) added, followed by cooling to 0° C. The TBTU (42 mg, 0.13 mmol) dissolved in 0.3 ml DMF is added and then the 4-(4-(2,3-dihydrobenzofuran-7-yl)piperazin-1-yl)butylamine (36 mg, 0.13 mmol) dissolved in 5 ml methylene chloride is added dropwise. The reaction mixture is agitated for 0.5 hour at ambient temperature. Then the deposit is shaken out several times with saturated sodium hydrogen carbonate solution and the combined aqueous phases are extracted again with methylene chloride. The collected organic phases are washed with saturated sodium chloride solution, dried with magnesium sulphate and concentrated in the rotary evaporator. The residue is purified by flash chromatography (CH₂Cl₂-MeOH: 98-2).

Yield: 51 mg (97%) white solid matter.

M.P.: 139° C. MS m/z 435 (M⁺). IR (NaCl) v (cm⁻¹): 3317; 2935; 2816; 1630; 1543; 1252; 1155; 756. ¹H NMR (CDCl₃, 360 MHz) δ (ppm): 1.65-1.73 (m, 4H, CH₂—CH₂); 2.48 (t, J=6.7 Hz, 2H, CH₂N(CH₂—CH₂)₂N); 2.61-2.66 (m, 4H, N(CH₂—CH₂)₂N); 3.17-3.22 (m, 6H, N(CH₂—CH₂)₂N, OCH₂CH₂); 3.48-3.53 (m, 2H, CH₂NHCO); 4.58 (t, J=8.3 Hz, 2H, OCH₂CH₂); 6.63 (d, J=7.5 Hz, 1H, H-phenyl); 6.71 (br t J=5.1 Hz, 1H, NHCO); 6.76-6.80 (m, 1H, H-phenyl); 6.86 (d, J=7.1 Hz, 1H, H-phenyl); 7.36-7.43 (m, 2H, H-5, H-6); 7.77 (s, 1H, H-3); 7.81 (d, J=7.5 Hz, 1H, H-4); 7.85 (d, J=7.3 Hz, 1H, H-7). ¹³C-NMR (CDCl₃, 90 MHz) δ (ppm): 24.2; 27.4; 30.0; 39.9; 49.4; 53.3; 57.9; 70.9; 115.6; 118.2; 121.0; 122.7; 124.8, 124.9, 125.0; 126.2; 127.5; 136.2 138.7; 139.1; 140.7; 151.1; 162.4.

C H N (%): C₂₅H₂₉N₃O₂S×0.25 H₂0

Calculated: C, 68.23; H, 6.76; N, 9.55; S, 7.28. Actual: C, 68.26; H, 6.64; N, 9.48; S, 7.36.

### EXAMPLE 2

### N-(4-(4-(2,3-dihydrobenzofuran-7-yl)piperazin-1-yl)butyl)indol-2-ylcarbamide

The synthesis take place analogously to example 1.

Yield: 40 mg (78%) white solid matter

M.P.: 154° C. MS m/z 418 (M⁺). IR (NaCl) v (cm⁻¹): 3415; 2927; 2854; 2817; 1635; 1556; 1250; 1070; 752. ¹H NMR (CDCl₃, 360 MHz) δ (ppm): 1.65-1.71 (m, 4H, CH₂—CH₂); 2.52 (t, J=6.9 Hz, 2H, CH₂N(CH₂—CH₂)₂N); 2.70-2.73 (m, 4H, N(CH₂—CH₂)₂N); 3.17-3.22 (m, 6H, O—CH₂—CH₂, N(CH₂—CH₂)₂N); 3.50-3.55 (m, 2H, CH₂NHCO); 4.59 (t, J=8.7 Hz, 2H, O—CH₂—CH₂); 6.67-6.70 (m, 2H, Phenyl, NHCO); 6.77-6.82 (m, 1H, Phenyl,); 6.86-6.87 (m, 2H, Phenyl, H-3); 7.11-7.15 (m, 1H, H-5); 7.25-7.30 (m, 1H, H-6); 7.44 (dd, J=8.3 Hz, J=0.7 Hz, 1H, H-7); 7.64 (d, J=8.0 Hz, 1H, H-4); 9.57 (brs, 1H, NH). ¹³C-NMR (CDCl₃, 90 MHz) δ (ppm): 24.2; 27.4; 30.0; 39.4; 49.2; 53.2; 57.9; 71.0; 102.0; 111.9; 115.7; 118.3; 120.6; 121.1, 121.9; 124.3; 127.5, 127.6; 130.9; 136.0, 136.2; 151.1; 161.7.

C H N (%): C₂₅H₃₀N₄O₂×0.1H₂O

Calculated: C, 71.40; H, 7.24; N, 13.32. Actual: C, 71.38; H, 7.22; N, 13.33.

### EXAMPLE 3

### N-(4-(4-(chroman-8-yl)piperazin-1-yl)butyl)benzo[b]thiophen-2-ylcarbamide

Benzo[b]thiophene-2-carboxylic acid (21 mg, 0.12 mmol) is dissolved in dry methylene chloride (4 ml) and 0.07 ml DIEA (0.42 mmol) added, followed by cooling to 0° C. The TBTU (42 mg, 0.13 mmol) dissolved in 0.5 ml DMF is added and then the 4-(4-(chroman-8-yl)piperazin-1-yl)butylamine (39 mg, 0.13 mmol) dissolved in 5 ml methylene chloride is added dropwise. The reaction mixture is agitated for 1 hour at ambient temperature, and then shaken out several times with saturated sodium hydrogen carbonate solution and the combined aqueous phases are extracted again with methylene chloride. The collected organic phases are washed with saturated sodium chloride solution, dried with magnesium sulphate and concentrated in the rotary evaporator. The residue is purified by means of flash chromatography (CH₂Cl₂-MeOH: 98-2).

Yield: 53 mg (98%) white solid matter.

M.P.: 134° C. MS: m/z 449 (M⁺). IR (NaCl) v (cm⁻¹): 3325; 2930; 2853; 2817; 1631; 1544; 1248; 1217; 754. ¹H NMR (CDCl₃, 360 MHz) δ (ppm): 1.69-1.74 (m, 4H, CH₂—CH₂); 1.96-2.03 (m, 2H, O—CH₂—CH₂—CH₂); 2.55 (t, J=6.9 Hz, 2H, CH₂N(CH₂—CH₂)₂N); 2.74-2.80 (m, 6H, N(CH₂—CH₂)₂N, O—CH₂—CH₂—CH₂); 3.09-3.12 (m, 4H, N(CH₂—CH₂)₂N); 3.48-3.53 (m, 2H, CH₂NHCO); 4.24 (t, J=5.1 Hz, 2H, O—CH₂—CH₂—CH₂); 6.69 (dd, 1H, J=7.1 Hz, J=2.5 Hz, H-phenyl); 6.72-6.80 (m, 2H, H-phenyl); 6.83 (brt, J=4.9 Hz, 1H, NHCO); 7.36-7.44 (m, 2H, H-5, H-6); 7.79 (brs, 1H, H-3); 7.81-7.86 (m, 2H, H-4, H-7). ¹³C NMR (CDCl₃, 90 MHz) δ (ppm): 22.1; 23.9; 25.1; 27.2; 39.8; 50.2; 53.4; 57.9; 66.5; 115.9; 119.9; 122.6; 122.7; 124.1; 124.8; 125.0; 125.1; 126.2; 139.7; 139.1; 140.4; 140.7; 147.6; 162.5.

C H N (%): C₂₆H₃₁N₃O₂S×0.45 H₂O

Calculated: C, 68.21; H, 7.03; N, 9.18; S, 7.00. Actual: C, 68.09; H, 6.87; N, 8.95; S, 7.05.

### EXAMPLE 4

### N-(4-(4-(chroman-8-yl)piperazin-1-yl)butyl)indol-2-ylcarbamide

The synthesis take place analogously to example 3.

Yield: 31 mg (61%) white solid matter

M.P.: 141° C. MS: m/z432 (M⁺). IR (NaCl) v (cm⁻¹): 3265; 2929; 2853; 2822; 1635; 1554; 1248; 1217. ¹H NMR (CDCl₃, 360 MHz) δ (ppm): 1.66-1.74 (m, 4H, CH₂—CH₂); 1.96-2.03 (m, 2H, O—CH₂—CH₂—CH₂); 2.50 (t, J=6.9 Hz, 2H, CH₂N(CH₂—CH₂)₂N); 2.63-3.72 (m, 4H, N(CH₂—CH₂)₂N); 2.78 (t, J=6.4 Hz, 2H, O—CH₂—CH₂—CH₂); 3.08-3.14 (m, 4H, N(CH₂—CH₂)₂N); 3.50-3.55 (m, 2H, CH₂NHCO); 4.25 (t, J=5.1 Hz, 2H, O—CH₂—CH₂—CH₂); 6.65 (brt, J=5.3 Hz, 1H, NHCO); 6.73 (dd, J=2.3 Hz, J=6.9 Hz, 1H, H-phenyl); 6.73-6.80 (m, 2H, H-phenyl); 6.86 (dd, J=2.2 Hz, J=0.7 Hz, 1H, H-3); 7.14 (dd J=7.5 Hz, J=1.2 Hz, 1H, H-5); 7.29 (dd, J=8.3 Hz, J=1.2 Hz, 1H, H-6); 7.44 (dd, J=8.3 Hz, J=0.8 Hz, 1H, H-7); 7.64 (dd, J=7.5 Hz, J=0.8 Hz, 1H, H-4); 9.46 (s, 1H, NH). ¹³C NMR (CDCl₃, 90 MHz) δ (ppm): 22.1; 24.2; 25.1; 27.4; 39.5; 50.4; 53.4; 57.9; 66.5; 101.9; 111.9; 115.9; 119.9; 120.6; 121.9; 122.7; 124.0, 124.4; 127.6; 130.9; 136.2; 140.7; 147.6; 161.7.

C H N (%): C₂₆H₃₂N₄O₂×0.6 H₂O

Calculated: C, 70.24; H, 7.56; N, 12.60. Actual: C, 70.58; H, 7.42; N, 12.22.

### EXAMPLE 5

### N-(4-(4-(2,3-dihydrobenzofuran-7-yl)piperazin-1-yl)butyl)benzo[b]thiophen-3-ylcarbamide

The synthesis take place analogously to example 1.

Yield: 51 mg (97%) white solid matter.

M.P.: 159° C. MS m/z 435 (M⁺). IR (NaCl) v (cm⁻¹): 3319; 2939; 2817; 1633; 1539; 1254; 1147; 764. ¹H NMR (CDCl₃, 360 MHz) δ (ppm): 1.66-1.75 (m, 4H, CH₂—CH₂); 2.46 (t, J=6.8 Hz, 2H, CH₂N(CH₂—CH₂)₂N); 2.59-2.61 (m, 4H, N(CH₂—CH₂)₂N); 3.03-3.03 (m, 6H, N(CH₂—CH₂)₂N); 3.19 (t, J=8.7 Hz, 2H, OCH₂CH₂); 3.49-3.55 (m, 2H, CH₂NHCO); 4.58 (t, J=8.9 Hz, 2H, OCH₂CH₂); 6.59 (d, J=7.5 Hz, 1H, H-phenyl); 6.76-6.83 (m, 2H, H-phenyl, NHCO); 6.85 (dd, J=7.5 Hz, J=1.1 Hz, 1H, H-phenyl); 7.36-7.46 (m, 2H, H-5, H-6); 7.83 (s, 1H, H-2); 7.84-7.87 (m, 1H, H-4); 8.34 (d, J=7.5 Hz, 1H, H-7).

### EXAMPLE 6

### N-(4-(4-(2,3-dihydrobenzofuran-7-yl)piperazin-1-yl)butyl)benzofuran-3-ylcarbamide

The synthesis take place analogously to example 1.

Yield: 41 mg (81%) colorless oil

(APCl) MS m/z 420 ((M+H)⁺). IR (NaCl) v (cm⁻¹): 3315; 2941; 2819; 1637; 1566; 1452; 1254; 1012; 752. ¹H NMR (CDCl₃, 360 MHz) δ (ppm): 1.65-1.75 (m, 4H, CH₂—CH₂); 2.48 (t, J=6.9 Hz, 2H, CH₂N(CH₂—CH₂)₂N); 2.62-2.66 (m, 4H, N(CH₂—CH₂)₂N); 3.09-3.13 (m, 4H, N(CH₂—CH₂)₂N); 3.19 (t, J=8.7 Hz, 2H, O—CH₂—CH₂); 3.50-3.54 (m, 2H, CH₂NHCO); 4.58 (t, J=8.9 Hz, 2H, O—CH₂—CH₂); 6.47 (brt, J=4.9 Hz, 1H, NHCO); 6.64 (dd, J=7.4 Hz, J=0.7 Hz, 1H, H-phenyl,); 6.76-6.80 (m, 1H, H-phenyl); 7.85 (dd, J=7.4 Hz, J=0.7 Hz, 1H, H-phenyl); 7.32-7.38 (m, 2H, H-5, H-6); 7.51-7.54 (m, 1H, H-7); 7.90-7.94 (m, 1H, H-4); 8.09 (s, 1H, H-2). ¹³C-NMR (CDCl₃, 90 MHz) δ (ppm): 24.3; 27.6; 30.1; 39.5; 49.4; 53.3; 58.1; 71.0; 111.9; 115.7; 118.1; 118.2; 121.0, 121.9; 123.9; 124.6; 125.2, 127.5; 136.2, 146.7; 151.1; 155.5; 163.0.

### EXAMPLE 7

### N-(4-(4-(2,3-dihydrobenzofuran-7-yl)piperazin-1-yl)butyl)indol-3-ylcarbamide

The synthesis take place analogously to example 1.

Yield: 39 mg (77%) colorless oil

MS m/z 418 (M⁺). IR (NaCl) v (cm⁻¹): 2927; 2856; 2817; 1631; 1547; 1251; 1007; 752. ¹H NMR (CDCl₃, 360 MHz) δ (ppm): 1.66-1.74 (m, 4H, CH₂—CH₂); 2.47 (t, J=6.9 Hz, 2H, CH₂N(CH₂—CH₂)₂N); 2.62-2.65 (m, 4H, N(CH₂—CH₂)₂N); 3.10-3.14 (m, 4H, N(CH₂—CH₂)₂N); 3.19 (t, J=8.7 Hz, 2H, O—CH₂—CH₂); 3.51-3.56 (m, 2H, CH₂NHCO); 4.58 (t, J=8.9 Hz, 2H, O—CH₂—CH₂); 6.27 (brt, J=5.0 Hz, 1H, NHCO); 6.65 (d, J=7.4 Hz, 1H, H-phenyl); 6.77-6.81 (m, 1H, H-phenyl); 6.85 (dd, J=7.4 Hz, J=0.9 Hz, 1H, H-phenyl); 7.22-7.26 (m, 2H, H-5, H-2); 7.41-7.46 (m, 1H, H-6); 7.74 (d, J=3.0 Hz, 1H, H-7 od. H-4); 7.94-7.98 (m, 1H, H-7 od. H-4); 8.94 (brs, 1H, NH).

### EXAMPLE 8

### N-(4-(4-(2,3-dihydrobenzofuran-7-yl)-1,4-diazepan-1-yl)butyl)benzo[b]thiophen-2-ylcarbamide

Benzo[b]thiophene-2-carboxylic acid (21 mg, 0.12 mmol) is converted with 4-(4-(2,3-dihydrobenzofuran-7-yl)-1,4-diazepan-1-yl)-butylamine (38 mg, 0.13 mmol) as described for example 1. Purification is by flash chromatography (CH₂Cl₂-MeOH: 95-5).

Yield: 42 mg (78%) colorless oil.

MS m/z 449 (M⁺). IR (NaCl) v (cm⁻¹): 2925; 2852; 1631; 1547; 1240; 756. ¹H NMR (CDCl₃, 360 MHz) δ (ppm): 1.70-1.78 (m, 4H, CH₂—CH₂); 2.10-2.16 (m, 2H, N(CH₂—CH₂)N(CH₂—CH₂—CH₂)N); 2.73 (t, J=6.9 Hz, 2H, CH₂N(CH₂—CH₂)N(CH₂—CH₂—CH₂)N); 2.92-2.95 (m, 2H, N(CH₂—CH₂)N(CH₂—CH₂—CH₂)N); 3.00-3.03 (m, 2H, N(CH₂—CH₂)N(CH₂—CH₂—CH₂)N); 3.17 (t, J=8.7 Hz, 2H, O—CH₂—CH₂); 3.39 (t J=6.5 Hz, 2H, N(CH₂—CH₂)N(CH₂—CH₂)N); 3.49-3.56 (m, 4H, CH₂NHCO, N(CH₂—CH₂)N(CH₂—CH₂—CH₂)N); 4.51 (t, J=8.7 Hz, 2H, OCH₂CH₂); 6.59 (dd, J=7.5 Hz, J=1.8 Hz, 1H, H-phenyl); 6.71-6.78 (m, 2H, H-phenyl); 7.08 (br t J=3.6 Hz, 1H, NHCO); 7.35-7.43 (m, 2H, H-5, H-6); 7.81-7.86 (m, 2H, H-4, H-7); 7.89 (brs, 1H, H-3). ¹³C-NMR (CDCl₃, 90 MHz) δ (ppm): 24.0; 26.8; 26.9; 30.2; 39.4; 49.5; 49.7; 53.8; 56.6; 56.8; 70.7; 114.5; 115.8; 121.1; 122.6; 124.7, 125.0; 125.1; 126.0; 127.5; 136.3 138.9; 139.2; 140.8; 149.2; 162.5.

### EXAMPLE 9

### N-(4-(4-(chroman-8-yl)-1,4-diazepan-1-yl)butyl)benzo[b]thiophen-3-ylcarbamide

Benzo[b]thiophene-3-carboxylic acid (21 mg, 0.12 mmol) is converted with 4-(4-(chroman-8-yl)-1,4-diazepan-1-yl)-butylamine (40 mg, 0.13 mmol) as described in example 1.

Purification is by flash chromatography (CH₂Cl₂-MeOH: 95-5).

Yield: 43 mg (77%) colorless oil.

(APCl)MS: m/z 464 ((M+H)⁺). IR (NaCl) v (cm⁻¹): 3292; 2929; 2859; 2817; 1635; 1539; 1217; 752. ¹H NMR (CDCl₃, 360 MHz) δ (ppm): 1.69-1.75 (m, 4H, CH₂—CH₂); 1.92-2.01 (m, 4H, O—CH₂—CH₂—CH₂, N(CH₂—CH₂)N(CH₂—CH₂—CH₂)N); 2.63 (t, J=6.7 Hz, 2H, CH₂N(CH₂—CH₂)N(CH₂—CH₂—CH₂)N); 2.77 (t, J=6.7 Hz, 2H, N(CH₂—CH₂)N(CH₂—CH₂—CH₂)N); 2.79-2.82 (m, 2H, N(CH₂—CH₂)N(CH₂—CH₂—CH₂)N); 2.86-2.89 (m, 2H, N(CH₂—CH₂)N(CH₂—CH₂—CH₂)N); 3.22-3.25 (m, 2H, O—CH₂—CH₂—CH₂); 3.50-3.54 (m, 2H, CH₂NHCO); 3.29-3.32 (m, 2H, N(CH₂—CH₂)N(CH₂—CH₂—CH₂)N); 4.17-4.20 (m, 2H, O—CH₂—CH₂—CH₂); 6.63 (dd, 1H, J=7.0 Hz, J=2.0 Hz, H-phenyl); 6.68-6.72 (m, 2H, H-phenyl); 7.08 (brt, J=5.0 Hz, 1H, NHCO); 7.35-7.46 (m, 2H, H-5, H-6); 7.83-7.86 (m, 1H, H-7); 7.91 (brs, 1H, H-2); 8.39-8.41 (m, 1H, H-4).

### EXAMPLE 10

### N-(4-(4-(chroman-8-yl)-1,4-diazepan-1-yl)butyl)benzo[b]thiophen-2-ylcarbamide

Benzo[b]thiophene-2-carboxylic acid (43 mg, 0.24 mmol) is converted with 4-(4-(chroman-8-yl)-1,4-diazepan-1-yl)-butylamine (82 mg, 0.26 mmol) as described for example 1.

Purification takes place by flash chromatography (CH₂Cl₂-MeOH: 95-5).

Yield: 101 mg (89%) colorless oil.

(APCl)MS: m/z 464 ((M+H)⁺). IR (NaCl) v (cm⁻¹): 3319; 2925; 2852; 2817; 1631; 1545; 1242; 1215; 752. ¹H NMR (CDCl₃, 360 MHz) δ (ppm): 1.68-1.72 (m, 4H, CH₂—CH₂); 1.94-2.06 (m, 4H, N(CH₂—CH₂)N(CH₂—CH₅—CH₂)N, CH₂N(CH₂—CH₂)N(CH₂—CH₂—CH₂)N); 2.67-2.72 (m, 2H, N(CH₂—CH₂)N(CH₂—CH₂—CH₂)N); 2.78 (t, J=6.2 Hz, 2H, O—CH₂—CH₂—CH₂); 2.89-2.92 (m, 2H, N(CH₂—CH₂)N(CH₂—CH₂—CH₂)N); 2.96-2.99 (m, 2H, N(CH₂—CH₂)N(CH₂—CH₂—CH₂)N); 3.27 (t, J=6.2 Hz, 2H, O—CH₂—CH₂); 3.29-3.32 (m, 2H, N(CH₂—CH₂)N(CH₂—CH₂—CH₂)N); 3.46-3.52 (m, 2H, CH₂NHCO); 4.16-4.19 (m, 2H, O—CH₂—CH₂—CH₂); 6.63 (dd, 1H, J=6.6 Hz, J=2.7 Hz, H-phenyl); 6.70-6.76 (m, 2H, H-phenyl); 7.13 (brt, J=4.7 Hz, 1H, NHCO); 7.33-7.41 (m, 2H, H-5, H-6); 7.73-7.75 (m, 1H, H-4); 7.78 (brs, 1H, H-3); 7.81-7.84 (m, 1H, H-7).

### EXAMPLE 11

### KS 478

### N-(4-(4-(chroman-8-yl)-1,4-diazepan-1-yl)butyl)benzofuran-2-ylcarbamide

Benzofuran-2-carboxylic acid (38 mg, 0.24 mmol) is converted with 4-(4-(chroman-8-yl)-1,4-diazepan-1-yl)-butylamine (79 mg, 0.26 mmol) as described for example 1. Purification takes place by flash chromatography (CH₂Cl₂-MeOH: 95-5).

Yield: 78 mg (72%) colorless oil.

(APCl)MS: m/z 448 ((M+H)⁺). IR (NaCl) v (cm⁻¹): 3305; 2939; 1657; 1595; 1520; 1219; 750. ¹H NMR (CDCl₃, 360 MHz) δ (ppm): 1.68-1.77 (m, 4H, CH₂—CH₂); 1.95-2.01 (m, 2H, O—CH₂—CH₂—CH₂,); 2.09-2.15 (m, 2H, N(CH₂—CH₂)N(CH₂—CH₂—CH₂)N); 2.78 (t, J=6.6 Hz, 2H, CH₂N(CH₂—CH₂)₂N); 2.89-2.92 (m, 2H, N(CH₂—CH₂)N(CH₂—CH₂—CH₂); 2.99-3.10 (m, 4H, N(CH₂—CH₂)N(CH₂—CH₂—CH₂)N); 3.29 (t, J=6.4 Hz, 2H, O—CH₂—CH₂—CH₂); 3.34-3.39 (m, 2H, N(CH₂—CH₂)N(CH₂—CH₂—CH₂)N); 3.48-3.53 (m, 2H, CH₂NHCO); 4.19 (t, J=5.2 Hz, 2H, O—CH₂—CH₂—CH₂); 6.64 (dd, 1H, J=6.1 Hz, J=3.1 Hz, H-phenyl); 6.72-6.74 (m, 2H, H-phenyl); 7.19-7.29 (m, 2H, NHCO, H-5); 7.38-7.40 (m, 1H, H-6); 7.42-7.44 (m, 2H, H-7, H-3); 7.63-7.66 (m, 1H, H-4).

### EXAMPLE 12

### N-(4-(4-(2,3,4,5-tetrahydrobenzo[b]oxepin-9-yl)piperazin-1-yl)butyl)benzo[b]thiophen-2-ylcarbamide

Benzo[b]thiophene-2-carboxylic acid (43 mg, 0.24 mmol) is converted with 4-(4-(2,3,4,5-tetrahydro-benzo[b]oxepin-9-yl)piperazin-1-yl)butylamine (90 mg, 0.30 mmol) as described for example 1. Purification takes place by flash chromatography (CH₂Cl₂-MeOH: 97-3).

Yield: 91 mg (82%) white solid matter.

M.P.: 124° C. MS: m/z 463 (M⁺). IR (NaCl) v (cm⁻¹): 3319; 2935; 2817; 1631; 1543; 1248; 1221; 754. ¹H NMR (CDCl₃, 360 MHz) δ (ppm): 1.70-1.80 (m, 6H, CH₂—CH₂, O—CH₂—CH₂—CH₂—CH₂); 1.96-2.01 (m, 2H, O—CH₂—CH₂—CH₂—CH₂); 2.66 (t, J=6.8 Hz, 2H, CH₂N(CH₂—CH₂)₂N); 2.80-2.85 (m, 6H, N(CH₂—CH₂)₂N, O—CH₂—CH₂—CH₂—CH₂); 3.17-3.20 (m, 4H, N(CH₂—CH₂)₂N); 3.53-3.58 (m, 2H, CH₂NHCO); 3.99-4.01 (m, 2H, O—CH₂—CH₂—CH₂—CH₂—CH₂); 6.76 (dd, 1H, J=7.8 Hz, J=1.7 Hz, H-phenyl); 6.83 (dd, 1H, J=7.5 Hz, J=1.6 Hz, H-phenyl); 6.87-6.94 (m 2H, H-phenyl, NHCO); 7.39-7.47 (m, 2H, H-5, H-6); 7.85 (brs, 1H, H-3); 7.86-7.89 (m, 2H, H-4, H-7). ¹³C NMR (CDCl₃, 90 MHz) δ (ppm): 23.6; 26.0; 27.1; 32.4; 34.4; 39.7; 50.3; 53.7; 57.9; 73.4; 116.9; 122.7; 123.5; 124.4; 124.8; 125.0; 125.1; 126.2; 137.1; 138.7; 139.2; 140.8; 144.1; 153.6; 162.6.

### EXAMPLE 13

### N-(4-(4-(2,3,4,5-tetrahydrobenzo[b]oxepin-9-yl)piperazin-1-yl)butyl)-5-cyanobenzo[b]thiophen-2-ylcarbamide

5-cyanobenzo[b]thiophene-2-carboxylic acid (21 mg, 0.12 mmol) is converted with 4-(4-(2,3,4,5-tetrahydrobenzo[b]oxepin-9-yl)piperazin-1-yl)butylamine (39 mg, 0.15 mmol) as described for example 1. Purification takes place by flash chromatography (CH₂Cl₂-MeOH: 97-3).

Yield: 26 mg (44%) colorless oil.

(APCl) MS: m/z 489 ((M+H)⁺). IR (NaCl) v (cm⁻¹): 3325; 2939; 2816; 2227; 1635; 1558; 1541; 1248; 752. ¹H NMR (CDCl₃, 360 MHz) δ (ppm): 1.70-1.80 (m, 6H, CH₂—CH₂, O—CH₂—CH₂—CH₂—CH₂); 1.96-2.02 (m, 2H, O—CH₂—CH₂—CH₂—CH₂); 2.51 (t, J=6.7 Hz, 2H, CH₂N(CH₂—CH₂)₂N); 2.69-2.71 (m, 4H, N(CH₂—CH₂)₂N); 2.80-2.83 (m, 2H, O—CH₂—CH₂CH₂—CH₂); 3.11-3.13 (m, 4H, N(CH₂—CH₂)₂N); 3.53-3.59 (m, 2H, CH₂NHCO); 3.98-4.01 (m, 2H, O—CH₂—CH₂—CH₂—CH₂); 6.68 (dd, 1H, J=7.8 Hz, J=1.7 Hz, H-phenyl); 6.79 (dd, 1H, J=7.5 Hz, J=1.6 Hz, H-phenyl); 6.87-6.94 (m 1H, H-phenyl); 7.05(brt, J=5.6 Hz, NHCO); 7.60 (dd, J=8.3 Hz, J=1.5 Hz, 1H, H-5); 7.82 (brs, 1H, H-3); 7.89 (dd, J=8.3 Hz, J=0.7 Hz, 1H, H-7); 8.18-8.19 (m, 1H, H-4).

### EXAMPLE 14

### N-(4-(4-(2,3,4,5-tetrahydrobenzo[b]oxepin-9-yl)piperazin-1-yl)butyl)benzofuran-2-ylcarbamide

Benzofuran-2-carboxylic acid (38 mg, 0.24 mmol) is converted with 4-(4-(2,3,4,5-tetrahydrobenzo[b]oxepin-9-yl)piperazin-1-yl)butylamine (90 mg, 0.30 mmol) as described for example 1. Purification takes place by flash chromatography (CH₂Cl₂-MeOH: 97-3).

Yield: 98 mg (91%) colorless oil.

(APCl) MS: m/z 448 ((M+H)⁺). IR (NaCl) v (cm⁻¹): 3313; 2935; 2816; 1655; 1595; 1520; 1250; 750. ¹H NMR (CDCl₃, 360 MHz) δ (ppm): 1.67-1.75 (m, 6H, CH₂—CH₂, O—CH₂—CH₂—CH₂—CH₂); 1.94-1.98 (m, 2H, O—CH₂—CH₂—CH₂—CH₂); 2.50 (t, J=7.0 Hz, 2H, CH₂N(CH₂—CH₂)₂N); 2.66-2.70 (m, 4H, N(CH₂—CH₂)₂N); 2.78-2.80 (m, 2H, O—CH₂—CH₂—CH₂—CH₂); 3.12-3.15 (m, 4H, N(CH₂—CH₂)₂N); 3.51-3.54 (m, 2H, CH₂NHCO); 3.97-3.99 (m, 2H, O—CH₂—CH₂—CH₂—CH₂); 6.77-6.79 (m, 2H, H-phenyl); 6.88-6.91 (m, 1H, H-phenyl); 7.05 (brt, J=5.3 Hz, 1H, NHCO); 7.27-7.29 (m, 1H, H-5); 7.38-7.41 (m, 1H, H-6); 7.46 (brs, 1H, H-3); 7.47-7.49 (m, 1H, H-4); 7.66-7.67 (m, 1H, H-7).

### EXAMPLE 15

### N-(4-(4-(2,3,4,5-tetrahydrobenzo[b]oxepin-9-yl)piperazin-1-yl)butyl)-5-bromobenzofuran-2-ylcarbamide

5-bromobenzo[b]furane-2-carboxylic acid (29 mg, 0.12 mmol) is converted with 4-(4-(2,3,4,5-tetrahydrobenzo[b]oxepin-9-yl)piperazin-1-yl)butylamine (39 mg, 0.15 mmol) as described for example 1. Purification takes place by flash chromatography (CH₂Cl₂-MeOH: 97-3).

Yield: 47 mg (74%) colorless oil.

(APCl) MS: m/z 527 (M+H⁺). IR (NaCl) v (cm⁻¹): 3319; 2931; 2816; 1653; 1595; 1518; 1248; 754. ¹H NMR (CDCl₃, 360 MHz) δ (ppm): 1.69-1.78 (m, 6H, CH₂—CH₂, O—CH₂—CH₂—CH₂—CH₂); 1.97-2.03 (m, 2H, O—CH₂—CH₉—CH₂—CH₂); 2.52 (t, J=6.3 Hz, 2H, CH₂N(CH₂—CH₂)₂N); 2.68-2.71 (m, 4H, N(CH₂—CH₂)₂N); 2.80-2.84 (m, 2H, O—CH₂—CH₂—CH₂—CH₂); 3.15-3.18 (m, 4H, N(CH₂—CH₂)₂N); 3.53-3.59 (m, 2H, CH₂NHCO); 4.00-4.03 (m, 2H, O—CH₂—CH₂—CH₂—CH₂); 6.75-6.78 (m, 2H, H-phenyl); 6.87-6.91 (m, 1H, H-phenyl); 7.10 (brt, J=6.2 Hz, 1H, NHCO); 7.35 (brd, J=8.9 Hz, 1H, H-6); 7.39 (d, J=0.9 Hz, 1H, H-3); 7.48 (d, J=1.8 Hz, J=8.9 Hz, 1H, H-7); 7.80 (d, J=1.8 Hz, 1H, H-4).

### EXAMPLE 16

### N-(4-(4-(2,3,4,5-tetrahydrobenzo[b]oxepin-9-yl)piperazin-1-yl)butyl)indol-2-ylcarbamide

Indole-2-carboxylic acid (19 mg, 0.12 mmol) is converted with 4-(4-(2,3,4,5-tetrahydrobenzo[b]oxepin-9-yl)piperazin-1-yl)butylamine (39 mg, 0.13 mmol) as described for example 1. Purification takes place by flash chromatography (CH₂Cl₂-MeOH: 95-5).

Yield: 43 mg (80%) colorless oil.

(APCl) MS: m/z 447 ((M+H)⁺). IR (NaCl) v (cm⁻¹): 3257; 2935; 2817; 1633; 1556; 1248; 733. ¹H NMR (CDCl₃, 360 MHz) δ (ppm): 1.71-1.82 (m, 6H, CH₂—CH₂, O—CH₂—CH₂—CH₂—CH₂); 1.97-2.03 (m, 2H, O—CH₂—CH₂—CH₂—CH₂); 2.63 (t, J=7.0 Hz, 2H, CH₂N(CH₂—CH₂)₂N); 2.78-2.84 (m, 6H, N(CH₂—CH₂)₂N, O—CH₂—CH₂—CH₂—CH₂); 3.21-3.23 (m, 4H, N(CH₂—CH₂)₂N); 3.55-3.61 (m, 2H, CH₂NHCO); 3.99-4.02 (m, 2H, O—CH₂—CH₂—CH₂—CH₂); 6.80 (dd, 1H, J=7.9 Hz, J=1.8 Hz, H-phenyl); 6.84 (dd, 1H, J=7.6 Hz, J=1.7 Hz, H-phenyl); 6.92-6.96 (m, 1H, H-phenyl); 6.97 (m, 1H, NHCO);6.99 (dd, J=0.9 Hz, J=2.0 Hz, H-3); 7.16 (ddd, J=8.0 Hz, J=7.0 Hz, J=1.1 Hz, 1H, H-5); 7.31 (ddd, J=8.0 Hz, J=7.0 Hz, J=1.1 Hz, 1H, H-6); 7.47 (dd, J=8.1 Hz, J=0.9 Hz, 1H. H-/); 7.68 (dd, J=8.1 Hz, J=0.9 Hz, 1H, H-4); 9.56 (brs, 1H, NH).

### EXAMPLE 17

### N-(4-(4-(2,3,4,5-tetrahydrobenzo[b]oxepin-9-yl)piperazin-1-yl)butyl)-6-cyanoindol-2-ylcarbamide

6-cyanoindole-2-carboxylic acid (22 mg, 0.12 mmol) is converted with 4-(4-(2,3,4,5-tetrahydrobenzo[b]oxepin-9-yl)piperazin-1-yl)butylamine (39 mg, 0.13 mmol) as described for example 1. Purification takes place by flash chromatography (CH₂Cl₂-MeOH: 95-5).

Yield: 36 mg (64%) colorless oil

(APCl) MS: m/z 472 ((M+H)⁺). IR (NaCl) v (cm⁻¹): 3234; 2935; 2817; 2220; 1641; 1552; 1323; 1248; 752. ¹H NMR (CDCl₃, 360 MHz) δ (ppm): 1.69-1.80 (m, 6H, CH₂—CH₂, O—CH₂—CH₂—CH₂—CH₂); 1.93-1.98 (m, 2H, O—CH₂—CH₂—CH₂—CH₂); 2.54 (t, J=6.7 Hz, 2H, CH₂N(CH₂—CH₂)₂N); 2.69-2.72 (m, 4H, N(CH₂—CH₂)₂N); 2.77-2.80 (m, 2H, O—CH₂—CH₂—CH₂CH₂); 3.12-3.15 (m, 4H, N(CH₂—CH₂)₂N); 3.58-3.63 (m, 2H, CH₂NHCO); 3.95-3.99 (m, 2H, O—CH₂—CH₂—CH₂—CH₂); 6.74 (dd, 1H, J=7.6 Hz, J=1.6 Hz, H-phenyl); 6.79 (dd, 1H, J=7.6 Hz, J=1.6 Hz, H-phenyl); 6.87-6.91 (m, 1H, H-phenyl); 6.96 (brs, 1H, H-3); 7.09 (brt, J=5.4 Hz, 1H, NHCO); 7.34 (dd, J=8.3 Hz, J=1.4 Hz, H-5); 7.70 (d, J=8.3 Hz, 1H, H-4); 7.83 (brs, 1H, H-7); 10.66 (brs, 1H, NH). ¹³C NMR (CDCl₃, 90 MHz) δ (ppm): 24.1; 26.1; 27.2; 32.5; 34.5; 39.6; 50.7; 53.7; 57.8; 73.4; 102.4; 106.7; 116.9; 117.3; 120.1; 122.8; 123.0; 123.5; 124.2; 130.6; 134.4; 135.1; 137.1; 144.4; 153.5; 161.1.

### EXAMPLE 18

### N-(4-(4-(2,3,4,5-tetrahydrobenzo[b]oxepin-9-yl)-1,4-diazepan-1-yl)butyl)benzo[b]thiophen-2-ylcarbamide

Benzo[b]thiophene-2-carboxylic acid (43 mg, 0.24 mmol) is converted with 4-(4-(2,3,4,5-tetrahydrobenzo[b]oxepin-9-yl)-1,4-diazepan-1-yl)butylamine (83 mg, 0.26 mmol) as described for example 1. Purification takes place by flash chromatography (CH₂Cl₂-MeOH: 95-5).

Yield: 101 mg (88%) colorless oil.

(APCl) MS: m/z 478 ((M+H)⁺). IR (NaCl) v (cm⁻¹): 3250; 2939; 2839; 1643; 1543; 750. ¹H NMR (CDCl₃, 360 MHz) δ (ppm): 1.69-1.73 (m, 2H, O—CH₂—CH₂—CH₂—CH₂); 1.78-1.82 (m 2H, CH₂—CH₂); 1.93-1.96 (m, 2H, O—CH₂—CH₂—CH₂—CH₂); 2.02-2.06 (m, 2H, CH₂—CH₂); 2.43-2.46 (m, 2H, N(CH₂—CH₂)N(CH₂—CH₂—CH₂)N); 2.77-2.79 (m, 2H, O—CH₂—CH₂—CH₂—CH₂); 3.09 (t, J=7.6 Hz, 2H, CH₂N(CH₂—CH₂)N(CH₂—CH₂—CH₂)N); 3.25-3.28 (m, 2H, N(CH₂—CH₂)N(CH₂—CH₂—CH₂)N); 3.36-3.41 (m, 4H, N(CH₂—CH₂)N(CH₂—CH₂—CH₂)N); 3.46-3.48 (m, 2H, N(CH₂—CH₂)N(CH₂—CH₂—CH₂)N); 3.55-3.59 (m, 2H, OCNH—CH₂); 3.88-3.91 (m, 2H, O—CH₂—CH₂—CH₂—CH₂); 6.74 (d, 1H, J=8.0 Hz, H-phenyl); 6.76 (d, 1H, J=8.0 Hz, H-phenyl); 6.87-6.90 (m, 1H, H-phenyl); 7.33-7.39 (m, 2H, H-5, H-6); 7.83 (d, J=7.9 Hz, 1H, H-7); 7.85 (d, J=7.6 Hz, 1H, H-4); 8.09 (brt, J=6.0 Hz, 1H, NHCO); 8.21 (s, 1H, H-3). ¹³C NMR (CDCl₃, 90 MHz) δ (ppm): 21.6; 24.4; 25.9; 26.0; 32.3; 34.5; 38.0; 49.3; 49.5; 52.6; 56.5; 73.5; 116.5; 122.5; 123.5; 123.7; 124.6; 125.3; 125.6; 126.0; 137.2; 139.3; 139.6; 141.1; 144.5; 152.3; 162.9.

### EXAMPLE 19

### N-(4-(4-(2,3,4,5-tetrahydrobenzo[b]oxepin-9-yl)-1,4-diazepan-1-yl)butyl)benzofuran-2-ylcarbamide

Benzofuran-2-carboxylic acid (39 mg, 0.24 mmol) is converted with 4-(4-(2,3,4,5-tetrahydrobenzo[b]oxepin-9-yl)-1,4-diazepan-1-yl)butylamine (83 mg, 0.26 mmol) as described for example 1. Purification takes place by flash chromatography (CH₂Cl₂-MeOH: 95-5).

Yield: 100 mg (90%) colorless oil.

(APCl) MS: m/z 462 ((M+H)⁺). IR (NaCl) v (cm⁻¹): 3271; 2931; 2864; 1653; 1593; 1470; 750. ¹H NMR (CDCl₃, 360 MHz) δ (ppm): 1.69-1.73 (m, 2H, CH₂—CH₂); 1.78-1.82 (m 2H, CH₂—CH₂); 1.93-1.96 (m, 2H, O—CH₂—CH₂—CH₂—CH₂); 2.02-2.06 (m, 2H, O—CH₂—CH₂—CH₂—CH₂); 2.43-2.46 (m, 2H, N(CH₂—CH₂)N(CH₂—CH₂—CH₂)N); 2.77-2.79 (m, 2H, O—CH₂—CH₂—CH₂—CH₂); 3.09 (t, J=7.6 Hz, 2H, CH₂—N(CH₂—CH₂)N(CH₂—CH₂—CH₂)N); 3.25-3.28 (m, 2H, N(CH₂—CH₂)N(CH₂—CH₂—CH₂)N); 3.36-3.41 (m, 4H, N(CH₂—CH₂)N(CH₂—CH₂—CH₂)N); 3.46-3.48 (m, 2H, N(CH₂—CH₂)N(CH₂—CH₂—CH₂)N); 3.55-3.59 (m, 2H, CH₂NHCO); 3.88-3.91 (m, 2H, O—CH₂—CH₂—CH₂—CH₂); 6.74 (d, 1H, J=8.3 Hz, H-phenyl); 6.76 (d, 1H, J=7.6 Hz, H-phenyl); 6.87-6.90 (m, 1H, H-phenyl); 7.33-7.39 (m, 2H, H-5, H-6); 7.83 (d, J=7.9 Hz, 1H, H-4); 7.85 (d, J=7.6 Hz, 1H, H-7); 8.09 (brt, J=6.0 Hz, 1H, NHCO); 8.21 (s, 1H, H-3). ¹³C NMR (CDCl₃, 90 MHz) δ (ppm): 21.7; 24.4; 26.0; 26.8; 32.3; 34.5; 38.0; 49.4; 49.6; 52.6; 56.9; 57.2; 73.4; 110.2; 111.9; 116.5; 122.6; 123.5; 123.5; 123.6; 126.8; 127.6; 137.2; 144.6; 148.6; 152.3; 154.8; 159.3.

### EXAMPLE 20

### N-(4-(4-(chroman-7-yl)piperazin-1-yl)butyl)benzo[b]thiophen-2-ylcarbamide

Benzo[b]thiophene-2-carboxylic acid (21 mg, 0.12 mmol) is converted with 4-(4-(chroman-7-yl)piperazin-1-yl)butylamine (38 mg, 0.13 mmol) as described for example 1. Purification takes place by flash chromatography (CH₂Cl₂-MeOH: 97-3).

Yield: 40 mg (74%) colorless oil.

(APCl) MS: m/z 450 ((M+H)⁺). IR (NaCl) v (cm⁻¹): 3284; 2929; 2856; 2817; 1624; 1512; 1294; 750. ¹H NMR (CDCl₃, 360 MHz) δ (ppm): 1.64-1.74 (m, 4H, CH₂—CH₂); 1.95-1.99 (m, 2H, O—CH₂—CH₂—CH₂); 2.46 (t, J=7.0 Hz, 2H, CH₂N(CH₂—CH₂)₂N); 2.59-2.62 (m, 4H, N(CH₂—CH₂)₂N); 2.70 (t, J=6.4, 2H, O—CH₂—CH₂—CH₂); 3.14-3.16 (m, 4H, N(CH₂—CH₂)₂N); 3.49-3.53 (m, 2H, CH₂NHCO); 4.14-4.16 (m, 2H, O—CH₂—CH₂—CH₂); 6.33 (d, J=2.4 Hz, 1H, H-phenyl); 6.44 (dd, J=8.3 Hz, J=2.4 Hz, 1H, H-phenyl); 6.56 (brt, J=4.0 Hz, 1H, NHCO); 6.90 (d, J=8.3 Hz, 1H, H-phenyl); 7.37-7.43 (m, 2H, H-5, H-6); 7.76 (brs, 1H, H-3); 7.81 (d, J=7.2 Hz, 1H, H-4); 7.84 (d, J=8.3 Hz, 1H, H-7). ¹³C NMR (CDCl₃, 90 MHz) δ (ppm): 22.6; 24.1; 24.6; 27.4; 40.0; 49.1; 53.2; 57.9; 66.5; 103.9; 109.0; 113.6; 122.7; 124.9; 125.0; 125.1; 126.2; 130.1; 138.6; 139.1; 140.7; 150.8; 155.3; 162.3.

### EXAMPLE 21

### N-(4-(4-(2,3-dihydrobenzofuran-5-yl)piperazin-1-yl)butyl)benzo[b]thiophen-2-ylcarbamide

Benzo[b]thiophene-2-carboxylic acid (43 mg, 0.24 mmol) is converted with 4-(4-(2,3-dihydrobenzofuran-5-yl)piperazin-1-yl)-butylamine (80 mg, 0.27 mmol) as described for example 1. Purification takes place by flash chromatography (CH₂Cl₂-MeOH: 97-3).

Yield: 100 mg (96%) colorless oil.

(APCl) MS m/z 436 ((M+H)⁺). IR (NaCl) v (cm⁻¹): 3070; 2937; 2814; 1626; 1543; 1491; 1217; 752. ¹H NMR (CDCl₃, 360 MHz) δ (ppm): 1.64-1.74 (m, 4H, CH₂—CH₂); 2.47 (t, J=7.2 Hz, 2H, CH₂N(CH₂—CH₂)₂N); 2.61-2.63 (m, 4H, N(CH₂—CH₂)₂N); 3.05-3.07 (m, 4H, N(CH₂—CH₂)₂N); 3.16 (t, J=8.3 Hz, OCH₂CH₂); 3.49-3.53 (m, 2H, CH₂NHCO); 4.52 (t, J=8.3 Hz, 2H, OCH₂CH₂); 6.62 (br t J=5.1 Hz, 1H, NHCO); 6.67-6.68 (m, 2H, H-phenyl); 6.82-6.83 (m, 1H, H-phenyl); 7.37-7.43 (m, 2H, H-5, H-6); 7.76 (brs, 1H, H-3); 7.81 (d, J=7.9 Hz, 1H, H-4); 7.84 (d, J=7.9 Hz, 1H, H-7). ¹³C-NMR (CDCl₃, 90 MHz) δ (ppm): 24.4; 27.5; 30.2; 40.0; 49.4; 51.2; 53.5; 57.9; 71.1; 109.1; 114.9; 117.1; 122.7; 124.8, 124.9, 125.0; 126.2; 127.6; 138.7; 139.1; 140.7; 146.0; 154.5; 162.4.

### EXAMPLE 22

### N-(4-(4-(chroman-6-yl)piperazin-1-yl)butyl)benzo[b]thiophen-2-ylcarbamide

Benzo[b]thiophene-2-carboxylic acid (42 mg, 0.24 mmol) is converted with 4-(4-(chroman-6-yl)piperazin-1-yl)butylamine (80 mg, 0.28 mmol) as described in example 1. Purification takes place by flash chromatography (CH₂Cl₂-MeOH: 97-3).

Yield: 100 mg (93%) colorless oil.

(APCl) MS: m/z 450 ((M+H)⁺). IR (NaCl) v (cm⁻¹): 3315; 2937; 2816; 1630; 1543; 1502; 1227; 754. ¹H NMR (CDCl₃, 360 MHz) δ (ppm): 1.65-1.74 (m, 4H, CH₂—CH₂); 1.96-1.99 (m, 2H, O—CH₂—CH₂—CH₂); 2.48 (t, J=7.0 Hz, 2H, CH₂N(CH₂—CH₂)₂N); 2.62-2.64 (m, 4H, N(CH₂—CH₂)₂N); 2.74 (t, J=6.4, 2H, O—CH₂—CH₂—CH₂); 3.07-3.09 (m, 4H, N(CH₂—CH₂)₂N); 3.49-3.53 (m, 2H, CH₂NHCO); 4.13 (t, J=5.1 Hz, 2H, O—CH₂—CH₂—CH₂); 6.59-6.60 (m, 1H, H-phenyl); 6.64 (brt, J=6.0 Hz, 1H, NHCO); 6.70-6.71 (m, 2H, H-phenyl); 7.37-7.43 (m, 2H, H-5, H-6); 7.76 (brs, 1H, H-3); 7.81 (d, J=7.6 Hz, 1H, H-4); 7.84 (d, J=7.9 Hz, 1H, H-7). ¹³C NMR (CDCl₃, 90 MHz) δ (ppm): 22.6; 24.3; 25.2; 27.4; 39.9; 50.5; 53.4; 57.9; 66.3; 116.8; 117.0; 118.2; 122.4; 122.7; 124.8; 124.9; 125.1; 126.2; 138.7; 139.1; 140.7; 145.0; 149.2; 162.4.

### EXAMPLE 23

### N-(4-(4-(2,3,4,5-tetrahydrobenzo[b]oxepin-7-yl)piperazin-1-yl)butyl)benzo[b]thiophen-2-ylcarbamide

Benzo[b]thiophene-2-carboxylic acid (42 mg, 0.24 mmol) is converted with 4-(4-(2,3,4,5-tetrahydrobenzo[b]oxepin-7-yl)piperazin-1-yl)butylamine (80 mg, 0.25 mmol) as described for example 1. Purification takes place by flash chromatography (CH₂Cl₂-MeOH: 97-3).

Yield: 54 mg (49%) colorless oil.

(APCl) MS: m/z 464 ((M+H)⁺). IR (NaCl) v (cm⁻¹): 3321; 2931; 2817; 1630; 1544; 1502; 1232; 771; 729. ¹H NMR (CDCl₃, 360 MHz) δ (ppm): 1.64-1.75 (m, 6H, CH₂—CH₂, O—CH₂—CH₂—CH₂—CH₂); 1.90-1.96 (m, 2H, O—CH₂—CH₂—CH₂—CH₂); 2.46 (t, J=6.9 Hz, 2H, CH₂N(CH₂—CH₂)₂N); 2.59-2.62 (m, 4H, N(CH₂—CH₂)₂N); 2.74-2.77 (m, 2H, O—CH₂—CH₂—CH₂—CH₂); 3.11-3.14 (m, 4H, N(CH₂—CH₂)₂N); 3.48-3.53 (m, 2H, CH₂NHCO); 3.92-3.95 (m, 2H, O—CH₂—CH₂—CH₂—CH₂); 6.61 (brt, J=5.1 Hz, 1H, NHCO); 6.64 (dd, 1H, J=8.5 Hz, J=3.0 Hz, H-phenyl); 6.68 (d, 1H, J=3.0 Hz, H-phenyl); 6.88 (d, J=8.5 Hz, 1H, H-phenyl); 7.36-7.44 (m, 2H, H-5, H-6); 7.76 (d, J=0.7 Hz, 1H, H-3); 7.79-7.85 (m, 2H, H-4, H-7).

### EXAMPLE 24

### N-(4-(4-(benzo[1,3]dioxol-4-yl)piperazin-1-yl)butyl)benzo[b]thiophen-2-ylcarbamide

Benzo[b]thiophene-2-carboxylic acid (42 mg, 0.24 mmol) is converted with 4-(4-(benzo[1,3]dioxol-4-yl)piperazin-1-yl)butylamine (70 mg, 0.25 mmol) as described for example 1. Purification takes place by flash chromatography (CH₂Cl₂-MeOH: 97-3).

Yield: 105 mg (99%) white solid matter.

M.P.: 184° C. (APCl) MS: m/z 438 ((M+H)⁺). IR (NaCl) v (cm⁻¹): 3315; 2935; 2883; 2814; 1630; 1539; 1452; 1255; 759. ¹H NMR (CDCl₃, 360 MHz) δ (ppm): 1.63-1.68 (m, 2H, CH₂—CH₂); 1.69-1.73 (m, 2H, CH₂—CH₂); 2.46 (t, J=7.2 Hz, 2H, CH₂N(CH₂—CH₂)₂N); 2.61-2.62 (m, 4H, N(CH₂—CH₂)₂N); 3.19-3.20 (m, 4H, N(CH₂—CH₂)₂N); 3.49-3.52 (m, 2H, CH₂NHCO); 5.92 (s, 2H, O—CH₂—O); 6.40 (dd, J=8.3 Hz, J=0.8 Hz, 1H, H-phenyl); 6.52 (dd, J=8.3 Hz, J=0.8 Hz, 1H, H-phenyl); 6.59 (brt, J=5.9 Hz, 1H, NHCO); 6.74-6.77 (m, 1H, H-phenyl); 7.37-7.43 (m, 2H, H-5, H-6); 7.76 (brs, 1H, H-3); 7.81 (d, J=7.9 Hz, 1H, H-4); 7.84 (d, J=7.9 Hz, 1H, H-7).

### EXAMPLE 25

### N-(4-(4-(benzo[1,3]dioxol-5-yl)piperazin-1-yl)butyl)benzo[b]thiophen-2-ylcarbamide

Benzo[b]thiophene-2-carboxylic acid (42 mg, 0.24 mmol) is converted with 4-(4-(benzo[1,3]dioxol-5-yl)piperazin-1-yl)butylamine (80 mg, 0.27 mmol) as described for example 1. Purification takes place by flash chromatography (CH₂Cl₂-MeOH: 97-3).

Yield: 96 mg (92%) white solid matter.

M.P.: 182-184° C. MS: m/z 437 (M⁺). IR (NaCl) v (cm⁻¹): 3313; 2939; 2877; 2819; 2773; 1620; 1549; 1502; 1221; 754. ¹H NMR (CDCl₃, 360 MHz) δ (ppm): 1.63-1.68 (m, 2H, CH₂—CH₂); 1.69-1.73 (m, 2H, CH₂—CH₂); 2.45 (t, J=7.0 Hz, 2H, CH₂N(CH₂—CH₂)₂N); 2.59-2.61 (m, 4H, N(CH₂—CH₂)₂N); 3.06-3.07 (m, 4H, N(CH₂—CH₂)₂N); 3.49-3.52 (m, 2H, CH₂NHCO); 5.89 (s, 2H, O—CH₂—O); 6.32 (dd, J=8.6 Hz, J=2.4 Hz, 1H, H-phenyl); 6.53 (d, J=2.4 Hz, 1H, H-phenyl); 6.60 (brt, J=4.5 Hz, 1H, NHCO); 6.70 (d, J=8.6 Hz, 1H, H-phenyl); 7.37-7.42 (m, 2H, H-5, H-6); 7.75 (brs, 1H, H-3); 7.80 (d, J=7.6 Hz, 1H, H-4); 7.84 (d, J=8.3 Hz, 1H, H-7). ¹³C NMR (CDCl₃, 90 MHz) δ (ppm): 24.4; 27.5; 40.1; 50.7; 53.3; 57.9; 99.9; 100.8; 108.1; 109.0; 122.7; 124.9; 124.93; 125.1; 126.2; 138.6; 139.1; 140.7; 141.6; 147.4; 148.2; 162.4.

### EXAMPLE 26

### N-(4-(4-(2,3-dihydrobenzo[1,4]dioxin-6-yl)piperazin-1-yl)butyl)benzo[b]thiophen-2-ylcarbamide

Benzo[b]thiophene-2-carboxylic acid (21 mg, 0.12 mmol) is converted with 4-(4-(2,3-dihydrobenzo[1,4]dioxin-6-yl)piperazin-1-yl)butylamine (50 mg, 0.17 mmol) as described for example 1. Purification takes place by flash chromatography (CH₂Cl₂-MeOH: 97-3).

Yield: 30 mg (56%) white solid matter.

M.P.: 141-143° C. (APCl) MS: m/z 452 ((M+H)⁺). IR (NaCl) v (cm⁻¹): 3278; 2966; 2935; 2871; 2823; 1618; 1564; 1508; 1224; 750. ¹H NMR (CDCl₃, 360 MHz) δ (ppm): 1.63-1.71 (m, 4H, CH₂—CH₂); 2.45 (t, J=6.8 Hz, 2H, CH₂N(CH₂—CH₂)₂N); 2.56-2.61 (m, 4H, N(CH₂—CH₂)₂N); 3.06-3.09 (m, 4H, N(CH₂—CH₂)₂N); 3.48-3.53 (m, 2H, CH₂NHCO); 4.19-4.25 (m, 4H, O—CH₂—CH₂—O); 6.42-4.44 (m, 2H, H-phenyl); 6.57 (brt, J=4.5 Hz, 1H, NHCO); 6.75-6.77 (m, 1H, H-phenyl); 7.36-7.44 (m, 2H, H-5, H-6); 7.75 (brs, 1H, H-3); 7.79-7.85 (m, 2H, H-4, H-7). ¹³C NMR (CDCl₃, 90 MHz) δ (ppm): 24.3; 27.4; 40.0; 50.1; 53.3; 57.9; 64.2; 64.6; 105.7; 110.3; 117.3; 122.7; 124.8; 124.9; 125.1; 126.2; 137.4; 138.6; 139.1; 140.7; 143.6; 146.4; 162.3.

### EXAMPLE 27

### N-(4-(4-(2,3-dihydrobenzo[1,4]dioxin-6-yl)-1,4-diazepan-1-yl)butyl)benzo[b]thiophen-2-ylcarbamide

Benzo[b]thiophene-2-carboxylic acid (18 mg, 0.10 mmol) is converted with 4-(4-(2,3-dihydrobenzo[1,4]dioxin-6-yl)-1,4-diazepan-1-yl)butylamine (39 mg, 0.13 mmol) as described for example 1. Purification takes place by flash chromatography (CH₂Cl₂-MeOH: 97-3).

Yield: 35 mg (75%) colorless oil.

MS: m/z 465 ((M+H)⁺). IR (NaCl) v (cm⁻): 2933; 2868; 2824; 1628; 1543; 1510; 1288; 754. ¹H NMR (CDCl₃, 360 MHz) δ (ppm): 1.59-1.71 (m, 4H, CH₂—CH₂); 1.94-2.00 (m, 2H, N(CH₂—CH₂)N(CH₂—CH₂—CH₂)N); 2.58 (t, J=6.2 Hz, 2H, CH₂N(CH₂—CH₂)N(CH₂—CH₂—CH₂)N); 2.67-2.70 (m, 2H, N(CH₂—CH₂)N(CH₂—CH₂—CH₂)N); 2.80-2.83 (m, 2H, N(CH₂—CH₂)N(CH₂—CH₂—CH₂)N); 3.39 (t, J=6.2 Hz, 2H, CH₂NHCO); 3.45-3.50 (m, 4H, N(CH₂—CH₂)N(CH₂—CH₂—CH₂)N); 4.16-4.24 (m, 4H, O—CH₂—CH₂—O); 6.18-6.21(m, 2H, H-phenyl); 6.64-6.75 (m, 2H, NHCO, H-phenyl); 7.36-7.44 (m, 2H, H-5, H-6); 7.78 (brs, 1H, H-3); 7.80-7.86 (m, 2H, H-4, H-7).

### EXAMPLE 28

### N-(4-(4-(3,4-dihydro-2H-benzo[b][1,4]dioxepin-7-yl)piperazin-1-yl)butyl)benzo[b]thiophen-2-ylcarbamide

Benzo[b]thiophene-2-carboxylic acid (43 mg, 0.24 mmol) is converted with 4-(4-(3,4-dihydro-2H-benzo[b][1,4]dioxepin-7-yl)piperazin-1-yl)butylamine (80 mg, 0.26 mmol) as described for example 1. Purification takes place by flash chromatography (CH₂Cl₂-MeOH: 97-3).

Yield: 105 mg (94%) white solid matter.

M.P.: 138° C. (APCl) MS: m/z 466 ((M+H)⁺). IR (NaCl) v (cm⁻¹): 3284; 2964; 2937; 2845; 1616; 1564; 1504; 1298; 1226; 750. ¹H NMR (CDCl₃, 360 MHz) δ (ppm): 1.66-1.73 (m, 4H, CH₂—CH₂); 2.13-2.17 (m, 2H, (O—CH₂—CH₂—CH₂—O); 2.52 (t, J=7.0 Hz, 2H, CH₂N(CH₂—CH₂)₂N); 2.65-2.67 (m, 4H, N(CH₂—CH₂)₂N); 3.13-3.14 (m, 4H, N(CH₂—CH₂)₂N); 3.49-3.52 (m, 2H, CH₂NHCO); 4.12 (t, J=5.8 Hz, 2H, O—CH₂—CH₂—CH₂—O); 4.17 (t, J=5.8 Hz, O—CH₂—CH₂—CH₂—O); 6.48 (dd, J=8.8 Hz, J=2.9 Hz, 1H, H-phenyl); 6.55 (d, J=2.9 Hz, 1H, H-phenyl); 6.68 (brt, J=5.3 Hz, 1H, NHCO); 6.88 (d, J=8.8 Hz, 1H, H-phenyl); 7.37-7.42 (m, 2H, H-5, H-6); 7.76 (brs, 1H, H-3); 7.80 (brd, J=7.2 Hz, 1H, H-4); 7.84 (brd, J=7.9 Hz, 1H, H-7). ¹³C NMR (CDCl₃, 90 MHz) δ (ppm): 23.8; 27.2; 32.3; 39.8; 49.3; 49.6; 53.1; 57.8; 70.7; 70.8; 109.7; 111.4; 121.9; 122.7; 124.9; 125.0; 125.1; 126.2; 138.7; 139.1; 140.8; 145.0; 147.4; 151.7; 162.4.

### EXAMPLE 136

### N-(4-(4-(2,3,4,5-tetrahydrobenzo[b]oxepin-9-yl)piperazin-1-yl)butyl)-3-chlorobenzo[b]thiophen-2-ylcarbamide

0.07 ml DIPEA (0.42 mmol) are added to a solution of 3-chlorobenzo[b]thiophene-2-carboxylic acid chloride (28 mg, 0.12 mmol) in dry methylene chloride (5 ml) and cooled to 0° C. Then 4-(4-(2,3,4,5-tetrahydrobenzo[b]oxepin-9-yl)piperazin-1-yl)butylamine (39 mg, 0.13 mmol) dissolved in 5 ml methylene chloride are added dropwise, the reaction mixture is agitated for 1 hour at ambient temperature, and then shaken out several times with saturated sodium hydrogen carbonate solution and the combined aqueous phases again extracted with methylene chloride. The collected organic phases are washed with saturated sodium chloride solution, dried with magnesium sulphate and concentrated in the rotary evaporator. The residue is purified by means of flash chromatography (CH₂Cl₂-MeOH: 90-10).

Yield: 40 mg (67%) colorless oil.

(APCl) MS: m/z 499 ((M+H)⁺). IR (NaCl) v (cm⁻¹): 3428; 2935; 2816; 1647; 1533; 1246; 752. ¹H NMR (CDCl₃, 600 MHz) δ (ppm): 1.67-1.77 (m, 6H, CH₂—CH₂, O—CH₂—CH₂CH₂—CH₂); 1.94-1.97 (m, 2H, O—CH₂—CH₂—CH₂—CH₂); 2.48-2.50 (m, 2H, CH₂N(CH₂—CH₂)₂N); 2.63-2.66 (m, 4H, N(CH₂—CH₂)₂N); 2.78-2.79 (m, 2H, O—CH₂—CH₂—CH₂CH₂); 3.06-3.09 (m, 4H, N(CH₂—CH₂)₂N); 3.55-3.58 (m, 2H, CH₂NHCO); 3.96-3.98 (m, 2H, O—CH₂CH₂—CH₂—CH₂); 6.71 (dd, 1H, J=7.8 Hz, J=1.3 Hz, H-phenyl); 6.77 (dd, 1H, J=7.8 Hz, J=1.3 Hz, H-phenyl); 6.86-6.89 (m 1H, H-phenyl); 7.34 (brt, J=4.5 Hz, NHCO); 7.48-7.50 (m, 2H, H-5, H-6); 7.86-7.88 (m, 1H, H-4).

### EXAMPLE 137

### N-(4-(4-(2,3,4,5-tetrahydrobenzo[b]oxepin-9-yl)piperazin-1-yl)butyl)-6-ethynylbenzo[b]thiophen-2-ylcarbamide

6-ethynylbenzo[b]thiophene-2-carboxylic acid (20 mg, 0.09 mmol) is converted with 4-(4-(2,3,4,5-tetrahydrobenzo[b]oxepin-9-yl)piperazin-1-yl)butylamine (33 mg, 0.10 mmol) as described for example 1. Purification takes place by flash chromatography (CH₂Cl₂-MeOH: 90-10).

Yield: 19 mg (43%) colorless oil.

(APCl) MS: m/z 488 ((M+H)⁺). IR (NaCl) v (cm⁻¹): 3291; 2927; 2856; 1633; 1562; 1277; 752. ¹H NMR (CDCl₃, 600 MHz) δ (ppm): 1.69-1.75 (m, 6H, CH₂—CH₂, O—CH₂—CH₂—CH₂—CH₂); 1.94-1.98 (m, 2H, O—CH₂—CH₂—CH₂—CH₂); 2.57-2.61 (m, 2H, CH₂N(CH₂—CH₂)₂N); 2.74-2.76 (m, 4H, N(CH₂—CH₂)₂N); 2.77-2.79 (m, 2H, O—CH₂—CH₂—CH₂—CH₂); 2.80 (s, 1H, CCH); 3.12-3.15 (m, 4H, N(CH₂—CH₂)₂N); 3.50-3.53 (m, 2H, CH₂NHCO); 3.96-3.98 (m, 2H, O—CH₂—CH₂—CH₂—CH₂); 6.71 (dd, 1H, J=7.7 Hz, J=1.5 Hz, H-phenyl); 6.80 (dd, 1H, J=7.7 Hz, J=1.5 Hz, H-phenyl); 6.88-6.92 (m 2H, H-phenyl, NHCO); 7.47 (dd, J=8.3 Hz, J=1.1 Hz, 1H, H-5); 7.74-7.78 (m, 2H, H-4, H-3), 7.99 (brs, 1H, H-7).

### EXAMPLE 158

### N-(4-(4-(6-chloro-3-oxo-3,4-dihydro-2H-benzo[1,4]oxazin-8-yl)piperazin-1-yl)butyl)benzo[b]thiophen-2-ylcarbamide

Benzo[b]thiophene-2-carboxylic acid (21 mg, 0.12 mmol) is converted with 8-(4-(4-aminobutyl)piperazin-1-yl)-6-chloro-3,4-dihydro-2H-benzo[1,4]oxazin-3-one (44 mg, 0.13 mmol) as described for example 1. Purification takes place by flash chromatography (CH₂Cl₂-MeOH: 95-5).

Yield: 47 mg (79%) colorless oil.

(APCl) MS: m/z 499 ((M+H)⁺). IR (NaCl) v (cm⁻¹): 2987; 2945; 1699; 1624; 1475; 1296; 1230; 750. ¹H NMR (CDCl₃, 360 MHz) δ (ppm): 1.66-1.76 (m, 4H, CH₂—CH₂); 2.49 (t, J=7.0 Hz, 2H, CH₂N); 2.60-2.63 (m, 4H, pip); 3.09-3.12 (m. 4H, pip); 3.51-3.56 (m, 2H, CH₂NHCO); 4.63 (s, 2H, O—CH₂—CONH); 6.53 (d, J=2.8 Hz, 1H, H-5′); 6.55 (d, J=2.8 Hz, 1H, H-7′); 6.82-6.85 (m, 1H, CH₂—NHCO); 7.39-7.45 (m, 2H, H-5, H-6); 7.81-7.87 (m, 3H, H-4, H-3, H-7); 8.99 (brs, 1H, NHCO—CH₂—O)

### Synthesis of Other Possible Embodiments

Further embodiments can be synthesised by coupling a heteroarene carboxylic acid of type A1 such as benzo[b]thiophene-2-carboxylic acid; 5-bromobenzo[b]thiophene-2-carboxylic acid; benzo[b]thiophene-3-carboxylic acid; benzofuran-2-carboxylic acid; indole-2-carboxylic acid; indole-3-carboxylic acid or of type A2 such as benzofuran-3-carboxylic acid; 6-cyanoindole-2-carboxylic acid; 5-cyanobenzo[b]thiophene-2-carboxylic acid or 6-ethynylbenzo[b]thiophene-2-carboxylic acid with the various examples for amine components, as they are described in detail above for types C1 to C8. The synthesis of the respective embodiments can take place here analogously to the instructions for the production of example 1. If embodiments such as the compounds of examples 138, 140, 142, 144, 146, 148, 150, 152, 154 or 156 are produced on the basis of 3-chlorobenzo[b]thiophene-2-carboxylic acid chloride and the amines of types C1 to C8, then the synthesis of the corresponding embodiments can take place analogously to the instructions of example 136.

Specific possible embodiments are:

### EXAMPLE 29

### N-(4-(4-(2,3-dihydrobenzofuran-7-yl)piperazin-1-yl)butyl)-5-cyanobenzo[b]thiophen-2-ylcarbamide

### EXAMPLE 30

### N-(4-(4-(2,3-dihydrobenzofuran-7-yl)piperazin-1-yl)butyl)benzofuran-2-ylcarbamide

### EXAMPLE 31

### N-(4-(4-(2,3-dihydrobenzofuran-7-yl)piperazin-1-yl)butyl)-5-bromobenzofuran-2-ylcarbamide

### EXAMPLE 32

### N-(4-(4-(2,3-dihydrobenzofuran-7-yl)piperazin-1-yl)butyl)-6-cyanoindol-2-ylcarbamide

### EXAMPLE 33

### N-(4-(4-(chroman-8-yl)piperazin-1-yl)butyl)-5-cyanobenzo[b]thiophen-2-ylcarbamide

### EXAMPLE 34

### N-(4-(4-(chroman-8-yl)piperazin-1-yl)butyl)benzofuran-2-ylcarbamide

### EXAMPLE 35

### N-(4-(4-(chroman-8-yl)piperazin-1-yl)butyl)-5-bromobenzofuran-2-ylcarbamide

### EXAMPLE 36

### N-(4-(4-(chroman-8-yl)piperazin-1-yl)butyl)benzofuran-3-ylcarbamide

### EXAMPLE 37

### N-(4-(4-(chroman-8-yl)piperazin-1-yl)butyl)-6-cyanoindol-2-ylcarbamide

### EXAMPLE 38

### N-(4-(4-(chroman-8-yl)piperazin-1-yl)butyl)indol-3-ylcarbamide

### EXAMPLE 39

### N-(4-(4-(2,3,4,5-tetrahydrobenzo[b]oxepin-9-yl)piperazin-1-yl)butyl)benzo[b]thiophen-3-ylcarbamide

### EXAMPLE 40

### N-(4-(4-(2,3,4,5-tetrahydrobenzo[b]oxepin-9-yl)piperazin-1-yl)butyl)benzofuran-3-ylcarbamide

### EXAMPLE 41

### N-(4-(4-(2,3,4,5-tetrahydrobenzo[b]oxepin-9-yl)piperazin-1-yl)butyl)indol-3-ylcarbamide

### EXAMPLE 42

### N-(4-(4-(2,3-dihydrobenzofuran-7-yl)-1,4-diazepan-1-yl)butyl)-5-cyanobenzo[b]thiophen-2-ylcarbamide

### EXAMPLE 43

### N-(4-(4-(2,3-dihydrobenzofuran-7-yl)-1,4-diazepan-1-yl)butyl)benzo[b]thiophen-3-ylcarbamide

### EXAMPLE 44

### N-(4-(4-(2,3-dihydrobenzofuran-7-yl)-1,4-diazepan-1-yl)butyl)benzofuran-2-ylcarbamide

### EXAMPLE 45

### N-(4-(4-(2,3-dihydrobenzofuran-7-yl)-1,4-diazepan-1-yl)butyl)-5-bromobenzofuran-2-ylcarbamide

### EXAMPLE 46

### N-(4-(4-(2,3-dihydrobenzofuran-7-yl)-1,4-diazepan-1-yl)butyl)benzofuran-3-ylcarbamide

### EXAMPLE 47

### N-(4-(4-(2,3-dihydrobenzofuran-7-yl)-1,4-diazepan-1-yl)butyl)indol-2-ylcarbamide

### EXAMPLE 48

### N-(4-(4-(2,3-dihydrobenzofuran-7-yl)-1,4-diazepan-1-yl)butyl)-6-cyanoindol-2-ylcarbamide

### EXAMPLE 49

### N-(4-(4-(2,3-dihydrobenzofuran-7-yl)-1,4-diazepan-1-yl)butyl)indol-3-ylcarbamide

### EXAMPLE 50

### N-(4-(4-(chroman-8-yl)-1,4-diazepan-1-yl)butyl)-5-cyanobenzo[b]thiophen-2-ylcarbamide

### EXAMPLE 51

### N-(4-(4-(chroman-8-yl)piperazin-1-yl)butyl)benzo[b]thiophen-3-ylcarbamide

### EXAMPLE 52

### N-(4-(4-(chroman-8-yl)-1,4-diazepan-1-yl)butyl)-5-bromobenzofuran-2-ylcarbamide

### EXAMPLE 53

### N-(4-(4-(chroman-8-yl)-1,4-diazepan-1-yl)butyl)benzofuran-3-ylcarbamide

### EXAMPLE 54

### N-(4-(4-(chroman-8-yl)-1,4-diazepan-1-yl)butyl)indol-2-ylcarbamide

### EXAMPLE 55

### N-(4-(4-(chroman-8-yl)-1,4-diazepan-1-yl)butyl)-6-cyanoindol-2-ylcarbamide

### EXAMPLE 56

### N-(4-(4-(chroman-8-yl)-1,4-diazepan-1-yl)butyl)indol-3-ylcarbamide

### EXAMPLE 57

### N-(4-(4-(2,3,4,5-tetrahydrobenzo[b]oxepin-9-yl)-1,4-diazepan-1-yl)butyl)-5-cyanobenzo[b]thiophen-2-ylcarbamide

### EXAMPLE 58

### N-(4-(4-(2,3,4,5-tetrahydrobenzo[b]oxepin-9-yl)-1,4-diazepan-1-yl)butyl)benzo[b]thiophen-3-ylcarbamide

### EXAMPLE 59

### N-(4-(4-(2,3,4,5-tetrahydrobenzo[b]oxepin-9-yl)-1,4-diazepan-1-yl)butyl)-5-bromobenzofuran-2-ylcarbamide

### EXAMPLE 60

### N-(4-(4-(2,3,4,5-tetrahydrobenzo[b]oxepin-9-yl)-1,4-diazepan-1-yl)butyl)benzofuran-3-ylcarbamide

### EXAMPLE 61

### N-(4-(4-(2,3,4,5-tetrahydrobenzo[b]oxepin-9-yl)-1,4-diazepan-1-yl)butyl)indol-2-ylcarbamide

### EXAMPLE 62

### N-(4-(4-(2,3,4,5-tetrahydrobenzo[b]oxepin-9-yl)-1,4-diazepan-1-yl)butyl)-6-cyanoindol-2-ylcarbamide

### EXAMPLE 63

### N-(4-(4-(2,3,4,5-tetrahydrobenzo[b]oxepin-9-yl)-1,4-diazepan-1-yl)butyl)indol-3-ylcarbamide

### EXAMPLE 64

### N-(4-(4-(benzo[1,3]dioxol-4-yl)piperazin-1-yl)butyl)-5-cyanobenzo[b]thiophen-2-ylcarbamide

### EXAMPLE 65

### N-(4-(4-(benzo[1,3]dioxol-4-yl)piperazin-1-yl)butyl)benzo[b]thiophen-3-ylcarbamide

### EXAMPLE 66

### N-(4-(4-(benzo[1,3]dioxol-4-yl)piperazin-1-yl)butyl)benzofuran-2-ylcarbamide

### EXAMPLE 67

### N-(4-(4-(benzo[1,3]dioxol-4-yl)piperazin-1-yl)butyl)-5-bromobenzofuran-2-ylcarbamide

### EXAMPLE 68

### N-(4-(4-(benzo[1,3]dioxol-4-yl)piperazin-1-yl)butyl)benzofuran-3-ylcarbamide

### EXAMPLE 69

### N-(4-(4-(benzo[1,3]dioxol-4-yl)piperazin-1-yl)butyl)indol-2-ylcarbamide

### EXAMPLE 70

### N-(4-(4-(benzo[1,3]dioxol-4-yl)piperazin-1-yl)butyl)-6-cyanoindol-2-ylcarbamide

### EXAMPLE 71

### N-(4-(4-(benzo[1,3]dioxol-4-yl)piperazin-1-yl)butyl)indol-3-ylcarbamide

### EXAMPLE 72

### N-(4-(4-(chroman-7-yl)piperazin-1-yl)butyl)-5-cyanobenzo[b]thiophen-2-ylcarbamide

### EXAMPLE 73

### N-(4-(4-(chroman-7-yl)piperazin-1-yl)butyl)benzo[b]thiophen-3-ylcarbamide

### EXAMPLE 74

### N-(4-(4-(chroman-7-yl)piperazin-1-yl)butyl)benzofuran-2-ylcarbamide

### EXAMPLE 75

### N-(4-(4-(chroman-7-yl)piperazin-1-yl)butyl)-5-bromobenzofuran-2-ylcarbamide

### EXAMPLE 76

### N-(4-(4-(chroman-7-yl)piperazin-1-yl)butyl)benzofuran-3-ylcarbamide

### EXAMPLE 77

### N-(4-(4-(chroman-7-yl)piperazin-1-yl)butyl)indol-2-ylcarbamide

### EXAMPLE 78

### N-(4-(4-(chroman-7-yl)piperazin-1-yl)butyl)-6-cyanoindol-2-ylcarbamide

### EXAMPLE 79

### N-(4-(4-(chroman-7-yl)piperazin-1-yl)butyl)indol-3-ylcarbamide

### EXAMPLE 80

### N-(4-(4-(benzo[1,3]dioxol-5-yl)piperazin-1-yl)butyl)-5-cyanobenzo[b]thiophen-2-ylcarbamide

### EXAMPLE 81

### N-(4-(4-(benzo[1,3]dioxol-5-yl)piperazin-1-yl)butyl)benzo[b]thiophen-3-ylcarbamide

### EXAMPLE 82

### N-(4-(4-(benzo[1,3]dioxol-5-yl)piperazin-1-yl)butyl)benzofuran-2-ylcarbamide

### EXAMPLE 83

### N-(4-(4-(benzo[1,3]dioxol-5-yl)piperazin-1-yl)butyl)-5-bromobenzofuran-2-ylcarbamide

### EXAMPLE 84

### N-(4-(4-(benzo[1,3]dioxol-5-yl)piperazin-1-yl)butyl)benzofuran-3-ylcarbamide

### EXAMPLE 85

### N-(4-(4-(benzo[1,3]dioxol-5-yl)piperazin-1-yl)butyl)indol-2-ylcarbamide

### EXAMPLE 86

### N-(4-(4-(benzo[1,3]dioxol-5-yl)piperazin-1-yl)butyl)-6-cyanoindol-2-ylcarbamide

### EXAMPLE 87

### N-(4-(4-(benzo[1,3]dioxol-5-yl)piperazin-1-yl)butyl)indol-3-ylcarbamide

### EXAMPLE 88

### N-(4-(4-(2,3-dihydrobenzo[1,4]dioxin-6-yl)piperazin-1-yl)butyl)-5-cyanobenzo[b]thiophen-2-ylcarbamide

### EXAMPLE 89

### N-(4-(4-(2,3-dihydrobenzo[1,4]dioxin-6-yl)piperazin-1-yl)butyl)benzo[b]thiophen-3-ylcarbamide

### EXAMPLE 90

### N-(4-(4-(2,3-dihydrobenzo[1,4]dioxin-6-yl)piperazin-1-yl)butyl)benzofuran-2-ylcarbamide

### EXAMPLE 91

### N-(4-(4-(2,3-dihydrobenzo[1,4]dioxin-6-yl)piperazin-1-yl)butyl)-5-bromobenzofuran-2-ylcarbamide

### EXAMPLE 92

### N-(4-(4-(2,3-dihydrobenzo[1,4]dioxin-6-yl)piperazin-1-yl)butyl)benzofuran-3-ylcarbamide

### EXAMPLE 93

### N-(4-(4-(2,3-dihydrobenzo[1,4]dioxin-6-yl)piperazin-1-yl)butyl)indol-2-ylcarbamide

### EXAMPLE 94

### N-(4-(4-(2,3-dihydrobenzo[1,4]dioxin-6-yl)piperazin-1-yl)butyl)-6-cyanoindol-2-ylcarbamide

### EXAMPLE 95

### N-(4-(4-(2,3-dihydrobenzo[1,4]dioxin-6-yl)piperazin-1-yl)butyl)indol-3-ylcarbamide

### EXAMPLE 96

### N-(4-(4-(3,4-dihydro-2H-benzo[b][1,4]dioxepin-7-yl)piperazin-1-yl)butyl)-5-cyanobenzo[b]thiophen-2-ylcarbamide

### EXAMPLE 97

### N-(4-(4-(3,4-dihydro-2H-benzo[b][1,4]dioxepin-7-yl)piperazin-1-yl)butyl)benzo[b]thiophen-3-ylcarbamide

### EXAMPLE 98

### N-(4-(4-(3,4-dihydro-2H-benzo[b][1,4]dioxepin-7-yl)piperazin-1-yl)butyl)benzofuran-2-ylcarbamid

### EXAMPLE 99

### N-(4-(4-(3,4-dihydro-2H-benzo[b][1,4]dioxepin-7-yl)piperazin-1-yl)butyl)-5-bromobenzofuran-2-ylcarbamide

### EXAMPLE 100

### N-(4-(4-(3,4-dihydro-2H-benzo[b][1,4]dioxepin-7-yl)piperazin-1-yl)butyl)benzofuran-3-ylcarbamid

### EXAMPLE 101

### N-(4-(4-(3,4-dihydro-2H-benzo[b][1,4]dioxepin-7-yl)piperazin-1-yl)butyl)indol-2-ylcarbamide

### EXAMPLE 102

### N-(4-(4-(3,4-dihydro-2H-benzo[b][1,4]dioxepin-7-yl)piperazin-1-yl)butyl)-6-cyanoindol-2-ylcarbamide

### EXAMPLE 103

### N-(4-(4-(3,4-dihydro-2H-benzo[b][1,4]dioxepin-7-yl)piperazin-1-yl)butyl)indol-3-ylcarbamide

### EXAMPLE 104

### N-(4-(4-(2,3-dihydrobenzo[1,4]dioxin-6-yl)-1,4-diazepan-1-yl)butyl)-5-cyanobenzo[b]thiophen-2-ylcarbamide

### EXAMPLE 105

### N-(4-(4-(2,3-dihydrobenzo[1,4]dioxin-6-yl)-1,4-diazepan-1-yl)butyl)benzo[b]thiophen-3-ylcarbamide

### EXAMPLE 106

### N-(4-(4-(2,3-dihydrobenzo[1,4]dioxin-6-yl)-1,4-diazepan-1-yl)butyl)benzofuran-2-ylcarbamide

### EXAMPLE 107

### N-(4-(4-(2,3-dihydrobenzo[1,4]dioxin-6-yl)-1,4-diazepan-1-yl)butyl)-5-bromobenzofuran-2-ylcarbamide

### EXAMPLE 108

### N-(4-(4-(2,3-dihydrobenzo[1,4]dioxin-6-yl)-1,4-diazepan-1-yl)butyl)benzofuran-3-ylcarbamide

### EXAMPLE 109

### N-(4-(4-(2,3-dihydrobenzo[1,4]dioxin-6-yl)-1,4-diazepan-1-yl)butyl)indol-2-ylcarbamide

### EXAMPLE 110

### N-(4-(4-(2,3-dihydrobenzo[1,4]dioxin-6-yl)-1,4-diazepan-1-yl)butyl)-6-cyanoindol-2-ylcarbamide

### EXAMPLE 111

### N-(4-(4-(2,3-dihydrobenzo[1,4]dioxin-6-yl)-1,4-diazepan-1-yl)butyl)indol-3-ylcarbamide

### EXAMPLE 112

### N-(4-(4-(2,3-dihydrobenzofuran-5-yl)piperazin-1-yl)butyl)-5-cyanobenzo[b]thiophen-2-ylcarbamide

### EXAMPLE 113

### N-(4-(4-(2,3-dihydrobenzofuran-5-yl)piperazin-1-yl)butyl)benzo[b]thiophen-3-ylcarbamide

### EXAMPLE 114

### N-(4-(4-(2,3-dihydrobenzofuran-5-yl)piperazin-1-yl)butyl)benzofuran-2-ylcarbamide

### EXAMPLE 115

### N-(4-(4-(2,3-dihydrobenzofuran-5-yl)piperazin-1-yl)butyl)-5-bromobenzofuran-2-ylcarbamide

### EXAMPLE 116

### N-(4-(4-(2,3-dihydrobenzofuran-5-yl)piperazin-1-yl)butyl)benzofuran-3-ylcarbamide

### EXAMPLE 117

### N-(4-(4-(2,3-dihydrobenzofuran-5-yl)piperazin-1-yl)butyl)indol-2-ylcarbamide

### EXAMPLE 118

### N-(4-(4-(2,3-dihydrobenzofuran-5-yl)piperazin-1-yl)butyl)-6-cyanoindol-2-ylcarbamide

### EXAMPLE 119

### N-(4-(4-(2,3-dihydrobenzofuran-5-yl)piperazin-1-yl)butyl)indol-3-ylcarbamide

### EXAMPLE 120

### N-(4-(4-(chroman-6-yl)piperazin-1-yl)butyl)-5-cyanobenzo[b]thiophen-2-ylcarbamide

### EXAMPLE 121

### N-(4-(4-(chroman-6-yl)piperazin-1-yl)butyl)benzo[b]thiophen-3-ylcarbamide

### EXAMPLE 122

### N-(4-(4-(chroman-6-yl)piperazin-1-yl)butyl)benzofuran-2-ylcarbamide

### EXAMPLE 123

### N-(4-(4-(chroman-6-yl)piperazin-1-yl)butyl)-5-bromobenzofuran-2-ylcarbamide

### EXAMPLE 124

### N-(4-(4-(chroman-6-yl)piperazin-1-yl)butyl)benzofuran-3-ylcarbamide

### EXAMPLE 125

### N-(4-(4-(chroman-6-yl)piperazin-1-yl)butylindol-2-ylcarbamide

### EXAMPLE 126

### N-(4-(4-(chroman-6-yl)piperazin-1-yl)butyl)-6-cyanoindol-2-ylcarbamide

### EXAMPLE 127

### N-(4-(4-(chroman-6-yl)piperazin-1-yl)butyl)indol-3-ylcarbamide

### EXAMPLE 128

### N-(4-(4-(2,3,4,5-tetrahydrobenzo[b]oxepin-7-yl)piperazin-1-yl)butyl)-5-cyanobenzo[b]thiophen-2-ylcarbamide

### EXAMPLE 129

### N-(4-(4-(2,3,4,5-tetrahydrobenzo[b]oxepin-7-yl)piperazin-1-yl)butyl)benzo[b]thiophen-3-ylcarbamide

### EXAMPLE 130

### N-(4-(4-(2,3,4,5-tetrahydrobenzo[b]oxepin-7-yl)piperazin-1-yl)butyl)benzofuran-2-ylcarbamide

### EXAMPLE 131

### N-(4-(4-(2,3,4,5-tetrahydrobenzo[b]oxepin-7-yl)piperazin-1-yl)butyl)-5-bromobenzofuran-2-ylcarbamide

### EXAMPLE 132

### N-(4-(4-(2,3,4,5-tetrahydrobenzo[b]oxepin-7-yl)piperazin-1-yl)butyl)benzofuran-3-ylcarbamide

### EXAMPLE 133

### N-(4-(4-(2,3,4,5-tetrahydrobenzo[b]oxepin-7-yl)piperazin-1-yl)butyl)indol-2-ylcarbamide

### EXAMPLE 134

### N-(4-(4-(2,3,4,5-tetrahydrobenzo[b]oxepin-7-yl)piperazin-1-yl)butyl)-6-cyanoindol-2-ylcarbamide

### EXAMPLE 135

### N-(4-(4-(2,3,4,5-tetrahydrobenzo[b]oxepin-7-yl)piperazin-1-yl)butyl)indol-3-ylcarbamide

### EXAMPLE 138

### N-(4-(4-(2,3-dihydrobenzofuran-7-yl)piperazin-1-yl)butyl)-3-chlorobenzo[b]thiophen-2-ylcarbamide

### EXAMPLE 139

### N-(4-(4-(2,3-dihydrobenzofuran-7-yl)piperazin-1-yl)butyl)-6-ethynylbenzo[b]thiophen-2-ylcarbamide

### EXAMPLE 140

### N-(4-(4-(chroman-8-yl)piperazin-1-yl)butyl)-3-chlorobenzo[b]thiophen-2-ylcarbamide

### EXAMPLE 141

### N-(4-(4-(chroman-8-yl)piperazin-1-yl)butyl)-6-ethynylbenzo[b]thiophen-2-ylcarbamide

### EXAMPLE 142

### N-(4-(4-(2,3-dihydrobenzofuran-7-yl)-1,4-diazepan-1-yl)butyl)-3-chlorobenzo[b]thiophen-2-ylcarbamide

### EXAMPLE 143

### N-(4-(4-(2,3-dihydrobenzofuran-7-yl)-1,4-diazepan-1-yl)butyl)-6-ethynylbenzo[b]thiophen-2-ylcarbamide

### EXAMPLE 144

### N-(4-(4-(chroman-8-yl)-1,4-diazepan-1-yl)butyl)-3-chlorobenzo[b]thiophen-2-ylcarbamide

### EXAMPLE 145

### N-(4-(4-(chroman-8-yl)-1,4-diazepan-1-yl)butyl)-6-ethynylbenzo[b]thiophen-2-ylcarbamide

### EXAMPLE 146

### N-(4-(4-(2,3,4,5-tetrahydrobenzo[b]oxepin-9-yl)-1,4-diazepan-1-yl)butyl)-3-chlorobenzo[b]thiophen-2-ylcarbamide

### EXAMPLE 147

### N-(4-(4-(2,3,4,5-tetrahydrobenzo[b]oxepin-9-yl)-1,4-diazepan-1-yl)butyl)-6-ethynylbenzo[b]thiophen-2-ylcarbamide

### EXAMPLE 148

### N-(4-(4-(benzo[1,3]dioxol-4-yl)piperazin-1-yl)butyl)-3-chlorobenzo[b]thiophen-2-ylcarbamide

### EXAMPLE 149

### N-(4-(4-(benzo[1,3]dioxol-4-yl)piperazin-1-yl)butyl)-6-ethynylbenzo[b]thiophen-2-ylcarbamide

### EXAMPLE 150

### N-(4-(4-(chroman-7-yl)piperazin-1-yl)butyl)-3-chlorobenzo[b]thiophen-2-ylcarbamide

### EXAMPLE 151

### N-(4-(4-(chroman-7-yl)piperazin-1-yl)butyl)-6-ethynylbenzo[b]thiophen-2-ylcarbamide

### EXAMPLE 152

### N-(4-(4-(benzo[1,3]dioxol-5-yl)piperazin-1-yl)butyl)-3-chlorobenzo[b]thiophen-2-ylcarbamide

### EXAMPLE 153

### N-(4-(4-(benzo[1,3]dioxol-5-yl)piperazin-1-yl)butyl)-6-ethynylbenzo[b]thiophen-2-ylcarbamide

### EXAMPLE 154

### N-(4-(4-(2,3-dihydrobenzo[1,4]dioxin-6-yl)piperazin-1-yl)butyl)-3-chlorobenzo[b]thiophen-2-ylcarbamide

### EXAMPLE 155

### N-(4-(4-(2,3-dihydrobenzo[1,4]dioxin-6-yl)piperazin-1-yl)butyl)-6-ethynylbenzo[b]thiophen-2-ylcarbamide

### EXAMPLE 156

### N-(4-(4-(3,4-dihydro-2H-benzo[b][1,4]dioxepin-7-yl)piperazin-1-yl)butyl)-3-chlorobenzo[b]thiophen-2-ylcarbamide

### EXAMPLE 157

### N-(4-(4-(3,4-dihydro-2H-benzo[b][1,4]dioxepin-7-yl)piperazin-1-yl)butyl)-6-ethynylbenzo[b]thiophen-2-ylcarbamide

### Biological Activity

The biological activities of the compounds according to the invention were determined in radioligand binding investigations. All radioligand experiments were performed according to methods described by us (Hübner, H. et al. J. Med. Chem. 2000, 43, 756-762). For the measurement of the affinities to the receptors of the D2-family membrane homogenates of Chinese hamster ovary cells (CHO cells) were used, which stably express the human D2long-, the human D2short- (Hayes, G. et al. Mol. Endocrinol. 1992, 6, 920-926), the human D3- (Sokoloff, P. et al. Eur. J. Pharmacol. 1992, 225, 331-337) or the human D4.4-receptor sub-type, (Asghari, V. J. Neurochem. 1995, 65, 1157-1165) respectively. Basically the binding assays took place by incubation of the receptor homogenates with the radioligand [³H]spiperone and the compounds under investigation in various concentrations. Determination of the affinities to the D1-receptor took place with native membrane homogenates, obtained from porcine striatum, and the D1-selective radioligands [³H]SCH 23390.

Measurement of the binding strengths of the compounds to the serotonin-receptor subtypes 5-HT1a and 5-HT2 was carried out according to methods described by us (Heindl, C. et al. Tetrahedron: Asymmetry 2003, 14, 3141-3152). For this we incubated porcine cortex-membrane preparations with the radioligands [³H]8-OH-DPAT (for 5-HT1a) or [³H]ketanserin (5-HT2) and the compounds in various concentrations. In the same way the affinity of the test compounds to the porcine α1-receptor was investigated, wherein porcine cortex-membrane preparations and the α1-selective radioligand [³H]prazosin were used.

All compounds investigated in the dopamine receptor-binding assay demonstrated good to very good affinities to the dopamine receptors with a clear binding preference to D2 and D3 subtypes. There is always a clear selectivity to the D3 receptor here, which for all the compounds 1-28 tested was in the picomolar or lower nanomloar range (<20 nM). (Table 1)

TABLE 1
Binding data for the embodiments according to formulae I and II
for the dopamine receptors porcineD1, humanD2long,
humanD2short, humanD3 and humanD4.4 and the porcine serotonin
receptors 5-HT1a and 5-HT2 and the adreno receptor alpha1^{a}

| | Receptor binding (Ki values in [nM]) | | | | | | | |
|---|---|---|---|---|---|---|---|---|
| No. | D1 | D2long | D2short | D3 | D4 | 5-HT1 | 5-HT2 | alpha1 |
| 1 | 260 | 73 | 50 | 0.31 | 65 | 18 | 100 | 4.2 |
| 2 | 300 | 45 | 27 | 0.16 | 22 | 30 | 110 | 2 |
| 3 | 300 | 110 | 83 | 0.35 | 50 | 17 | 77 | 4.6 |
| 4 | 290 | 42 | 22 | 0.17 | 34 | 20 | 110 | 2.3 |

^{a}determined as the average value from 2-6 individual experiments in each case performed in triplicate

A comparison of the compounds known from the prior art (WO 2004/004729) demonstrates the superior D3-bonding of the compounds according to the invention (Table 1b):

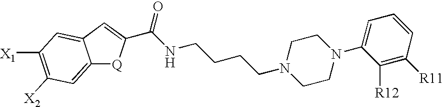

TABLE 1a
Q = NH; X1 = H

| | | | Substance according to the | |
|---|---|---|---|---|
| | Comparative | D3-bonding | invention; R11 + R12 form ring | D3-bonding |
| X2 | substance | (Ki in [nM]) | system | (Ki in [nM]) |
| CN | R11, R12 = chlorine | 0.35 | Tetrahydrobenzoxepine | 0.075 |
| CN | R11 = H; R12 = OCH3 | 0.25 | | |
| H | R11, R12 = chlorine | 0.56 | Dihydrobenzofuran | 0.35 |
| H | | | Chromane | 0.17 |
| H | | | Tetrahydrobenzoxepine | 0.052 |

TABLE 1b
Q = O; X2 = H

| | | | Substance according to the | |
|---|---|---|---|---|
| | Comparative | D3-bonding | invention; R11 + R12 form ring | D3-bonding |
| X1 | substance | (Ki in [nM]) | system | (Ki in [nM]) |
| Br | R11, R12 = chlorine | 3.4 | Tetrahydrobenzoxepine | 0.11 |
| Br | R11 = H; R12 = OCH3 | 0.69 | | |
| H | R11, R12 = chlorine | 1.2-1.5 | Tetrahydrobenzoxepine | 0.089 |
| H | R11 = H; R12 = OCH3 | 1.1 | | |

TABLE 1c
Q = S; X2 = H

| | | | Substance according to the | |
|---|---|---|---|---|
| | Comparative | D3-bonding | invention; R11 + R12 form ring | D3-bonding |
| X1 | substance | (Ki in [nM]) | system | (Ki in [nM]) |
| CN | R11, R12 = chlorine | 0.25 | Tetrahydrobenzoxepine | 0.097 |
| CN | R11 = H; R12 = OCH3 | 0.46 | | |
| H | R11, R12 = chlorine | 0.50 | Dihydrobenzofuran | 0.31 |
| H | R11 = H; R12 = OCH3 | 0.23 | Chromane | 0.16 |
| H | | | Tetrahydrobenzoxepine | 0.098 |

Investigations to determine the intrinsic activity of the example compounds were carried out in a mitogenesis assay in accordance with the literature (Hübner, H. et al. J. Med. Chem. 2000, 43, 4563-4569; Bettinetti, L. et al. J. Med. Chem. 2002, 45, 4594-4597). Here various concentrations of the compounds under investigation were incubated with D3 receptor-expressing cells and then the receptor-mediated stimulation of the mitogenesis rate was measured by incorporation of the radioactive marker [³H]thymidine. Agonistic, partial agonistic or antagonistic effects were determined in comparison with the effect of the full agonist quinpirol. (Table 2)

TABLE 2
Results of the mitogenesis experiments with the embodiments
at the dopamine D3 receptor to determine the intrinsic activity^{a}

| Compounds | EC₅₀-Wert [nM]^{b} | Agonistic activity [%]^{c} |
|---|---|---|
| Example 1 | 3.2 | 42 |
| Example 2 | — | 0 |
| Example 3 | 1.8 | 38 |
| Example 4 | — | 0 |
| Quinpirol | 3.2 | 100 |

^{a}dose-dependent incorporation of the radiomarker [³H]thymidine as a measure of the stimulation of the mitogenesis rate measured at seven different concentrations in quadruplicate
^{b}EC₅₀-value of the dose-effect curve derived from the average values of all individual trials
^{c}agonistic activity in [%] with reference to the maximum effect of the full agonist quinpirol

1. A compound of general formula I, 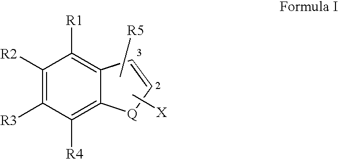
a physiologically acceptable salt thereof, or an enantiomer or diastereomer thereof, wherein
Q is selected from S, O and NR;
R is selected from hydrogen, alkyl, phenyl, alkylcarbonyl, phenylalkylcarbonyl, phenylcarbonyl, phenylalkyl and phenylsulfonyl;
R1, R2, R3 and R4 are independently selected from the group consisting of hydrogen, hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino;
R5 is a group bonded to position 2 or 3 of the bicyclic heteroaryl, selected from the group consisting of hydrogen, alkyl, halogen, alkoxy and amino;
X is a group of general formula X1 bonded at position 2 or 3 of the bicyclic heteroaryl 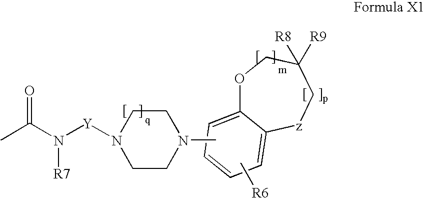
wherein:
R6 is selected from the group consisting of hydrogen, hydroxy, alkyl, alkyloxy, alkylthio, alkenyl, alkynyl, phenyl, phenylalkyl, phenoxy, halogen, trifluoromethyl, alkylcarbonyl, phenylcarbonyl, phenylalkylcarbonyl, alkyloxycarbonyl, phenylalkyloxycarbonyl, cyano, nitro, amino, carboxy, sulfo, sulfamoyl, sulfonylamino, alkylaminosulfonyl and alkylsulfonylamino;
R7 is hydrogen, alkyl or phenylalkyl;
Y is an unbranched, saturated or unsaturated hydrocarbon chain with 2-5 carbon atoms;
m and p are and independently 0, 1, or 2, wherein the sum of m and p is at most 2;
q is 1 or 2;
Z is CH₂, NH or O; and
R8 and R9 are independently selected from the group consisting of hydrogen, alkyl and phenyl or R8 and R9 together form an oxo-group;

2. The compound according to claim 1, wherein X is of general formula X2

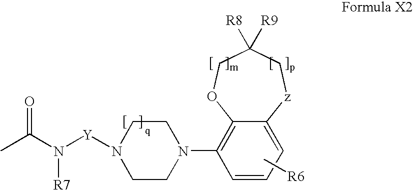

3. The compound according to claim 1, wherein R8 and R9 are hydrogen.

4. The compound according to claim 1, wherein R6 and R7 are hydrogen.

5. The compound according to claim 1, wherein Z is a —CH₂— or O.

6. The compound according to claim 1, wherein Z is NH.

7. The compound according to claim 1, wherein the sum of m and p is 1 or 2.

8. The compound according to claim 1, wherein R1, R4, R5, R6 and R7 in are hydrogen, and Y is a saturated, unbranched carbon chain with 4 or 5 carbons.

9. The compound according to claim 1 wherein q is 1.

10. The compound according to claim 1, wherein
Q is S, O or NH;
R1 and R4 are H;
R5 is H or halogen;
R2 and R3 are independently selected from the group consisting of H; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups and the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups and the C1-C6 alkyl is unsubstituted or hydroxy substituted; C1-C6 alkylaminosulfonyl, methylaminosulfonyl and C1-C6 alkylsulfonylamino; methanesulfonylamino; and
X is a group of formula X1 or X2, wherein
R6 is hydrogen, C1-C6 alkyl, C1-C6 alkoxy, or halogen;
R7 is hydrogen or C1-C6 alkyl;
R8 and R9 are hydrogen;
Z is CH₂ or O;
the sum of m and p is 0, 1 or 2;
q is 1 or 2; and
Y is an unbranched, saturated hydrocarbon chain with 3, 4 or 5 C-atoms.

11. The compound according to claim 1 having general formula II 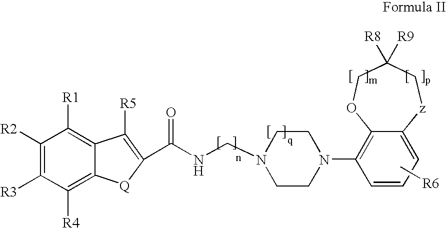
wherein n has the value of 3, 4 or 5.

12. The compound according to claim 1, having general formula III, IV, V, VI or VII 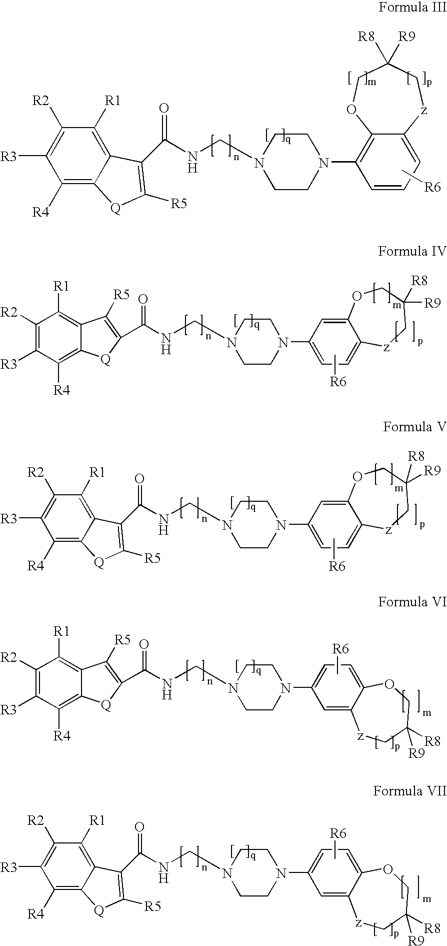
wherein n is 3, 4 or 5.

13. The compound according to claim 11, wherein Z is CH₂ or O.

14. The compound according to claim 11, wherein the sum of m and p is 1 or 2.

15. The compound according to claim 11, wherein R1, R4 and R6 are hydrogen.

16. The compound according to claim 11, wherein R8 and R9 are hydrogen.

17. The compound according to claim 11, wherein q is 1.

18. The compound according to claim 11, wherein:
R1, R4, R6, R8 and R9 are hydrogen;
R2 and R3 are independently selected from the group consisting of hydrogen; hydroxy; fluorine; chlorine; bromine; trifluoromethyl; cyano; amino; carboxy; sulfo; sulfamoyl; unsubstituted or hydroxy substituted C1-C6 alkyl; unsubstituted or hydroxy substituted C1-C6 alkyloxy; unsubstituted or hydroxy substituted C1-C6 alkylthio; unsubstituted C2-C6 alkynyl; unsubstituted phenyl or phenyl substituted with fluorine, chlorine or bromine and/or with one or more methoxy groups; phenyl(C1-C6)alkyl, wherein the phenyl is unsubstituted or substituted with fluorine, chlorine or bromine and/or with one or more methoxy groups and the C1-C6 alkyl is unsubstituted or hydroxy substituted; unsubstituted phenoxy or phenoxy substituted with fluorine, chlorine or bromine and/or with one or more methoxy groups; —C(O)—(C1-C6)alkyl, wherein the alkyl is unsubstituted or hydroxy substituted; —C(O)-phenyl, wherein the phenyl is unsubstituted or substituted with fluorine, chlorine or bromine and/or with one or more methoxy groups; —C(O)—(C1-C6)alkyl-phenyl, wherein the phenyl is unsubstituted or substituted with fluorine, chlorine or bromine and/or with one or more methoxy groups and the C1-C6 alkyl is unsubstituted or hydroxy substituted; C1-C6 alkyloxycarbonyl, wherein the alkyl is unsubstituted or hydroxy substituted; phenyl(C1-C6)alkyloxycarbonyl, wherein the phenyl is unsubstituted or substituted with fluorine, chlorine or bromine and/or with one or more methoxy groups and the C1-C6 alkyl is unsubstituted or hydroxy substituted; C1-C6 alkylaminosulfonyl, methylaminosulfonyl, C1-C6 alkylsulfonylamino; and methanesulfonylamino;
R5 is hydrogen or halogen;
n is 4 or 5;
q is 1 or 2; and
Z is CH₂ or O.

19. The compound according to claim 11, selected from the group consisting of
N-(4-(4-(2,3-dihydrobenzofuran-7-yl)piperazine-1-yl)butyl)benzo[b]thiophene-2-ylcarbamide,
N-(4-(4-(2,3-dihydrobenzofuran-7-yl)piperazine-1-yl)butyl)-3-chlorobenzo[b]thiophene-2-ylcarbamide,
N-(4-(4-(2,3-dihydrobenzofuran-7-yl)piperazine-1-yl)butyl)-5-cyanobenzo[b]thiophene-2-ylcarbamide,
N-(4-(4-(2,3-dihydrobenzofuran-7-yl)piperazine-1-yl)butyl)-6-ethynylbenzo[b]thiophene-2-ylcarbamide,
N-(4-(4-(2,3-dihydrobenzofuran-7-yl)piperazine-1-yl)butyl)benzofuran-2-ylcarbamide,
N-(4-(4-(2,3-dihydrobenzofuran-7-yl)piperazine-1-yl)butyl)-5-bromobenzofuran-2-ylcarbamide,
N-(4-(4-(2,3-dihydrobenzofuran-7-yl)piperazine-1-yl)butyl)indol-2-ylcarbamide,
N-(4-(4-(2,3-dihydrobenzofuran-7-yl)piperazine-1-yl)butyl)-6-cyanoindol-2-ylcarbamide,
N-(4-(4-(2,3-dihydrobenzofuran-7-yl)-1,4-diazepane-1-yl)butyl)benzo[b]thiophene-2-ylcarbamide,
N-(4-(4-(2,3-dihydrobenzofuran-7-yl)-1,4-diazepane-1-yl)butyl)-3-chlorobenzo[b]thiophene-2-ylcarbamide,
N-(4-(4-(2,3-dihydrobenzofuran-7-yl)-1,4-diazepane-1-yl)butyl)-5-cyanobenzo[b]thiophene-2-ylcarbamide,
N-(4-(4-(2,3-dihydrobenzofuran-7-yl)-1,4-diazepane-1-yl)butyl)-6-ethynylbenzo[b]thiophene-2-ylcarbamide,
N-(4-(4-(2,3-dihydrobenzofuran-7-yl)-1,4-diazepane-1-yl)butyl)benzofuran-2-ylcarbamide,
N-(4-(4-(2,3-dihydrobenzofuran-7-yl)-1,4-diazepane-1-yl)butyl)-5-bromobenzofuran-2-ylcarbamide,
N-(4-(4-(2,3-dihydrobenzofuran-7-yl)-1,4-diazepane-1-yl)butyl)indol-2-ylcarbamide,
N-(4-(4-(2,3-dihydrobenzofuran-7-yl)-1,4-diazepane-1-yl)butyl)-6-cyanoindol-2-ylcarbamide,
N-(4-(4-(chroman-8-yl)piperazin-1-yl)butyl)benzo[b]thiophene-2-ylcarbamide,
N-(4-(4-(chroman-8-yl)piperazin-1-yl)butyl)-3-chlorobenzo[b]thiophene-2-ylcarbamide,
N-(4-(4-(chroman-8-yl)piperazin-1-yl)butyl)-5-cyanobenzo[b]thiophene-2-ylcarbamide,
N-(4-(4-(chroman-8-yl)piperazin-1-yl)butyl)-6-ethynylbenzo[b]thiophene-2-ylcarbamide,
N-(4-(4-(chroman-8-yl)piperazin-1-yl)butyl)benzofuran-2-ylcarbamide,
N-(4-(4-(chroman-8-yl)piperazin-1-yl)butyl)-5-bromobenzofuran-2-ylcarbamide,
N-(4-(4-(chroman-8-yl)piperazin-1-yl)butyl)indol-2-ylcarbamide,
N-(4-(4-(chroman-8-yl)piperazin-1-yl)butyl)-6-cyanoindol-2-ylcarbamide,
N-(4-(4-(chroman-8-yl)-1,4-diazepan-1-yl)butyl)benzo[b]thiophene-2-ylcarbamide,
N-(4-(4-(chroman-8-yl)-1,4-diazepan-1-yl)butyl)-3-chlorobenzo[b]thiophene-2-ylcarbamide,
N-(4-(4-(chroman-8-yl)-1,4-diazepan-1-yl)butyl)-5-cyanobenzo[b]thiophene-2-ylcarbamide,
N-(4-(4-(chroman-8-yl)-1,4-diazepan-1-yl)butyl)-6-ethynylbenzo[b]thiophene-2-ylcarbamide,
N-(4-(4-(chroman-8-yl)-1,4-diazepan-1-yl)butyl)benzofuran-2-ylcarbamide,
N-(4-(4-(chroman-8-yl)-1,4-diazepan-1-yl)butyl)-5-bromobenzofuran-2-ylcarbamide,
N-(4-(4-(chroman-8-yl)-1,4-diazepan-1-yl)butyl)indol-2-ylcarbamide,
N-(4-(4-(chroman-8-yl)-1,4-diazepan-1-yl)butyl)-6-cyanoindol-2-ylcarbamide,
N-(4-(4-(2,3,4,5-tetrahydrobenzo[b]oxepin-9-yl)piperazine-1-yl)butyl)benzo[b]thiophene-2-ylcarbamide,
N-(4-(4-(2,3,4,5-tetrahydrobenzo[b]oxepin-9-yl)piperazin-1-yl)butyl)-3-chlorobenzo[b]thiophene-2-ylcarbamide,
N-(4-(4-(2,3,4,5-tetrahydrobenzo[b]oxepin-9-yl)piperazin-1-yl)butyl)-5-cyanobenzo[b]thiophene-2-ylcarbamide,
N-(4-(4-(2,3,4,5-tetrahydrobenzo[b]oxepin-9-yl)piperazin-1-yl)butyl)-6-ethinylbenzo[b]thiophene-2-ylcarbamide,
N-(4-(4-(2,3,4,5-tetrahydrobenzo[b]oxepin-9-yl)piperazin-1-yl)butyl)benzofuran-2-ylcarbamide,
N-(4-(4-(2,3,4,5-tetrahydrobenzo[b]oxepin-9-yl)piperazin-1-yl)butyl)-5-bromobenzofuran-2-ylcarbamide,
N-(4-(4-(2,3,4,5-tetrahydrobenzo[b]oxepin-9-yl)piperazin-1-yl)butyl)indol-2-ylcarbamide,
N-(4-(4-(2,3,4,5-tetrahydrobenzo[b]oxepin-9-yl)piperazin-1-yl)butyl)-6-cyanoindol-2-ylcarbamide,
N-(4-(4-(2,3,4,5-tetrahydrobenzo[b]oxepin-9-yl)-1,4-diazepan-1-yl)butyl)benzo[b]thiophene-2-ylcarbamide,
N-(4-(4-(2,3,4,5-tetrahydrobenzo[b]oxepin-9-yl)-1,4-diazepan-1-yl)butyl)-3-chlorobenzo[b]thiophene-2-ylcarbamide,
N-(4-(4-(2,3,4,5-tetrahydrobenzo[b]oxepin-9-yl)-1,4-diazepan-1-yl)butyl)-5-cyanobenzo[b]thiophene-2-ylcarbamide,
N-(4-(4-(2,3,4,5-tetrahydrobenzo[b]oxepin-9-yl)-1,4-diazepan-1-yl)butyl)-6-ethynylbenzo[b]thiophene-2-ylcarbamide,
N-(4-(4-(2,3,4,5-tetrahydrobenzo[b]oxepin-9-yl)-1,4-diazepan-1-yl)butyl)benzofuran-2-ylcarbamide,
N-(4-(4-(2,3,4,5-tetrahydrobenzo[b]oxepin-9-yl)-1,4-diazepan-1-yl)butyl)-5-bromobenzofuran-2-ylcarbamide,
N-(4-(4-(2,3,4,5-tetrahydrobenzo[b]oxepin-9-yl)-1,4-diazepan-1-yl)butyl)indol-2-ylcarbamide,
N-(4-(4-(2,3,4,5-tetrahydrobenzo[b]oxepin-9-yl)-1,4-diazepan-1-yl)butyl)-6-cyanoindol-2-ylcarbamide,
N-(4-(4-(benzo[1,3]dioxol-4-yl)piperazin-1-yl)butyl)benzo[b]thiophene-2-ylcarbamide,
N-(4-(4-(benzo[1,3]dioxol-4-yl)piperazin-1-yl)butyl)-3-chlorobenzo[b]thiophene-2-ylcarbamide,
N-(4-(4-(benzo[1,3]dioxol-4-yl)piperazin-1-yl)butyl)-5-cyanobenzo[b]thiophene-2-ylcarbamide,
N-(4-(4-(benzo[1,3]dioxol-4-yl)piperazin-1-yl)butyl)-6-ethynylbenzo[b]thiophene-2-ylcarbamide,
N-(4-(4-(benzo[1,3]dioxol-4-yl)piperazin-1-yl)butyl)benzofuran-2-ylcarbamide,
N-(4-(4-(benzo[1,3]dioxol-4-yl)piperazin-1-yl)butyl)-5-bromobenzofuran-2-ylcarbamide,
N-(4-(4-(benzo[1,3]dioxol-4-yl)piperazin-1-yl)butyl)indol-2-ylcarbamide.
N-(4-(4-(benzo[1,3]dioxol-4-yl)piperazin-1-yl)butyl)-6-cyanoindol-2-ylcarbamide,
N-(4-(4-(6-chloro-3-oxo-3,4-dihydro-2H-benzo[1,4]oxazin-8-yl)piperazin-1-yl)butyl)benzo[b]thiophene-2-ylcarbamide, and
physiologically acceptable salts thereof.

20. The compound according to claim 12, selected from the group consisting of
(a) a compound of formula III selected from the group consisting of
N-(4-(4-(2,3-dihydrobenzofuran-7-yl)piperazin-1-yl)butyl)benzo[b]thiophen-3-ylcarbamide,
N-(4-(4-(2,3-dihydrobenzofuran-7-yl)piperazin-1-yl)butyl)benzofuran-3-ylcarbamide,
N-(4-(4-(2,3-dihydrobenzofuran-7-yl)piperazin-1-yl)butyl)indol-3-ylcarbamide,
N-(4-(4-(2,3-dihydrobenzofuran-7-yl)-1,4-diazepane-1-yl)butyl)benzo[b]thiophen-3-ylcarbamide,
N-(4-(4-(2,3-dihydrobenzofuran-7-yl)-1,4-diazepan-1-yl)butyl)benzofuran-3-ylcarbamide,
N-(4-(4-(2,3-dihydrobenzofuran-7-yl)-1,4-diazepan-1-yl)butyl)indol-3-ylcarbamide,
N-(4-(4-(chroman-8-yl)piperazin-1-yl)butyl)benzo[b]thiophen-3-ylcarbamide,
N-(4-(4-(chroman-8-yl)piperazin-1-yl)butyl)benzofuran-3-ylcarbamide,
N-(4-(4-(chroman-8-yl)piperazin-1-yl)butyl)indol-3-ylcarbamide,
N-(4-(4-(chroman-8-yl)-1,4-diazepan-1-yl)butyl)benzo[b]thiophen-3-ylcarbamide,
N-(4-(4-(chroman-8-yl)-1,4-diazepan-1-yl)butyl)benzofuran-3-ylcarbamide,
N-(4-(4-(chroman-8-yl)-1,4-diazepan-1-yl)butyl)indol-3-ylcarbamide,
N-(4-(4-(2,3,4,5-tetrahydrobenzo[b]oxepin-9-yl)piperazin-1-yl)butyl)benzo[b]thiophen-3-ylcarbamide,
N-(4-(4-(2,3,4,5-tetrahydrobenzo[b]oxepin-9-yl)piperazin-1-yl)butyl)benzofuran-3-ylcarbamide,
N-(4-(4-(2,3,4,5-tetrahydrobenzo[b]oxepin-9-yl)piperazin-1-yl)butyl)indol-3-ylcarbamide,
N-(4-(4-(2,3,4,5-tetrahydrobenzo[b]oxepin-9-yl)-1,4-diazepan-1-yl)butyl)benzo[b]thiophen-3-ylcarbamide,
N-(4-(4-(2,3,4,5-tetrahydrobenzo[b]oxepin-9-yl)-1,4-diazepan-1-yl)butyl)benzofuran-3-ylcarbamide,
N-(4-(4-(2,3,4,5-tetrahydrobenzo[b]oxepin-9-yl)-1,4-diazepan-1-yl)butyl)indol-3-ylcarbamide,
N-(4-(4-(benzo[1,3]dioxol-4-yl)piperazin-1-yl)butyl)benzo[b]thiophen-3-ylcarbamide,
N-(4-(4-(benzo[1,3]dioxol-4-yl)piperazin-1-yl)butyl)benzofuran-3-ylcarbamide,
N-(4-(4-(benzo[1,3]dioxol-4-yl)piperazin-1-yl)butyl)indol-3-ylcarbamide, and
physiologically acceptable salts thereof;
(b) a compound of formula IV selected from the group consisting of
N-(4-(4-(chroman-7-yl)piperazin-1-yl)butyl)benzo[b]thiophen-2-ylcarbamide,
N-(4-(4-(chroman-7-yl)piperazin-1-yl)butyl)-3-chlorobenzo[b]thiophen-2-ylcarbamide,
N-(4-(4-(chroman-7-yl)piperazin-1-yl)butyl)-5-cyanobenzo[b]thiophen-2-ylcarbamide,
N-(4-(4-(chroman-7-yl)piperazin-1-yl)butyl)-6-ethynylbenzo[b]thiophen-2-ylcarbamide,
N-(4-(4-(chroman-7-yl)piperazin-1-yl)butyl)benzofuran-2-ylcarbamide,
N-(4-(4-(chroman-7-yl)piperazin-1-yl)butyl)-5-bromobenzofuran-2-ylcarbamide,
N-(4-(4-(chroman-7-yl)piperazin-1-yl)butyl)indol-2-ylcarbamide,
N-(4-(4-(chroman-7-yl)piperazin-1-yl)butyl)-6-cyanoindol-2-ylcarbamide,
N-(4-(4-(benzo[1,3]dioxol-5-yl)piperazin-1-yl)butyl)benzo[b]thiophen-2-ylcarbamide,
N-(4-(4-(benzo[1,3]dioxol-5-yl)piperazin-1-yl)butyl)-3-chlorobenzo[b]thiophen-2-ylcarbamide,
N-(4-(4-(benzo[1,3]dioxol-5-yl)piperazin-1-yl)butyl)-5-cyanobenzo[b]thiophen-2-ylcarbamide,
N-(4-(4-(benzo[1,3]dioxol-5-yl)piperazin-1-yl)butyl)-6-ethynylbenzo[b]thiophen-2-ylcarbamide,
N-(4-(4-(benzo[1,3]dioxol-5-yl)piperazin-1-yl)butyl)benzofuran-2-ylcarbamide,
N-(4-(4-(benzo[1,3]dioxol-5-yl)piperazin-1-yl)butyl)-5-bromobenzofuran-2-ylcarbamide,
N-(4-(4-(benzo[1,3]dioxol-5-yl)piperazin-1-yl)butyl)indol-2-ylcarbamide,
N-(4-(4-(benzo[1,3]dioxol-5-yl)piperazin-1-yl)butyl)-6-cyanoindol-2-ylcarbamide,
N-(4-(4-(2,3-dihydrobenzo[1,4]dioxin-6-yl)piperazin-1-yl)butyl)benzo[b]thiophen-2-ylcarbamide,
N-(4-(4-(2,3-dihydrobenzo[1,4]dioxin-6-yl)piperazin-1-yl)butyl)-3-chlorobenzo[b]thiophen-2-ylcarbamide,
N-(4-(4-(2,3-dihydrobenzo[1,4]dioxin-6-yl)piperazin-1-yl)butyl)-5-cyanobenzo[b]thiophen-2-ylcarbamide,
N-(4-(4-(2,3-dihydrobenzo[1,4]dioxin-6-yl)piperazin-1-yl)butyl)-6-ethynylbenzo[b]thiophen-2-ylcarbamide,
N-(4-(4-(2,3-dihydrobenzo[1,4]dioxin-6-yl)piperazin-1-yl)butyl)benzofuran-2-ylcarbamide,
N-(4-(4-(2,3-dihydrobenzo[1,4]dioxin-6-yl)piperazin-1-yl)butyl)-5-bromobenzofuran-2-ylcarbamide,
N-(4-(4-(2,3-dihydrobenzo[1,4]dioxin-6-yl)piperazin-1-yl)butyl)indol-2-ylcarbamide,
N-(4-(4-(2,3-dihydrobenzo[1,4]dioxin-6-yl)piperazin-1-yl)butyl)-6-cyanoindol-2-ylcarbamide,
N-(4-(4-(2,3-dihydrobenzo[1,4]dioxin-6-yl)-1,4-diazepan-1-yl)butyl)benzo[b]thiophen-2-ylcarbamide,
N-(4-(4-(2,3-dihydrobenzo[1,4]dioxin-6-yl)-1,4-diazepan-1-yl)butyl)-5-cyanobenzo[b]thiophen-2-ylcarbamide,
N-(4-(4-(2,3-dihydrobenzo[1,4]dioxin-6-yl)-1,4-diazepan-1-yl)butyl)benzofuran-2-ylcarbamide,
N-(4-(4-(2,3-dihydrobenzo[1,4]dioxin-6-yl)-1,4-diazepan-1-yl)butyl)-5-bromobenzofuran-2-ylcarbamide,
N-(4-(4-(2,3-dihydrobenzo[1,4]dioxin-6-yl)-1,4-diazepan-1-yl)butyl)indol-2-ylcarbamide,
N-(4-(4-(2,3-dihydrobenzo[1,4]dioxin-6-yl)-1,4-diazepan-1-yl)butyl)-6-cyanoindol-2-ylcarbamide,
N-(4-(4-(3,4-dihydro-2H-benzo[b][1,4]dioxepin-7-yl)piperazin-1-yl)butyl)benzo[b]thiophen-2-ylcarbamide,
N-(4-(4-(3,4-dihydro-2H-benzo[b][1,4]dioxepin-7-yl)piperazin-1-yl)butyl)-3-chlorobenzo[b]thiophen-2-ylcarbamide,
N-(4-(4-(3,4-dihydro-2H-benzo[b][1,4]dioxepin-7-yl)piperazin-1-yl)butyl)-5-cyanobenzo[b]thiophen-2-ylcarbamide,
N-(4-(4-(3,4-dihydro-2H-benzo[b][1,4]dioxepin-7-yl)piperazin-1-yl)butyl)-6-ethynylbenzo[b]thiophen-2-ylcarbamide,
N-(4-(4-(3,4-dihydro-2H-benzo[b][1,4]dioxepin-7-yl)piperazin-1-yl)butyl)benzofuran-2-ylcarbamide,
N-(4-(4-(3,4-dihydro-2H-benzo[b][1,4]dioxepin-7-yl)piperazin-1-yl)butyl)-5-bromobenzofuran-2-ylcarbamide,
N-(4-(4-(3,4-dihydro-2H-benzo[b][1,4]dioxepin-7-yl)piperazin-1-yl)butyl)indol-2-ylcarbamide,
N-(4-(4-(3,4-dihydro-2H-benzo[b][1,4]dioxepin-7-yl)piperazin-1-yl)butyl)-6-cyanoindol-2-ylcarbamide, and
physiologically acceptable salts thereof;
(c) a compound of formula V selected from the group consisting of
N-(4-(4-(chroman-7-yl)piperazin-1-yl)butyl)benzo[b]thiophen-3-ylcarbamide,
N-(4-(4-(chroman-7-yl)piperazin-1-yl)butyl)benzofuran-3-ylcarbamide,
N-(4-(4-(chroman-7-yl)piperazin-1-yl)butyl)indol-3-ylcarbamide,
N-(4-(4-(benzo[1,3]dioxol-5-yl)piperazin-1-yl)butyl)benzo[b]thiophen-3-ylcarbamide,
N-(4-(4-(benzo[1,3]dioxol-5-yl)piperazin-1-yl)butyl)benzofuran-3-ylcarbamide,
N-(4-(4-(benzo[1,3]dioxol-5-yl)piperazin-1-yl)butyl)indol-3-ylcarbamide,
N-(4-(4-(2,3-dihydrobenzo[1,4]dioxin-6-yl)piperazin-1-yl)butyl)benzo[b]thiophen-3-ylcarbamide,
N-(4-(4-(2,3-dihydrobenzo[1,4]dioxin-6-yl)piperazin-1-yl)butyl)benzofuran-3-ylcarbamide,
N-(4-(4-(2,3-dihydrobenzo[1,4]dioxin-6-yl)piperazin-1-yl)butyl)indol-3-ylcarbamide,
N-(4-(4-(2,3-dihydrobenzo[1,4]dioxin-6-yl)-1,4-diazepan-1-yl)butyl)benzo[b]thiophen-3-ylcarbamide,
N-(4-(4-(2,3-dihydrobenzo[1,4]dioxin-6-yl)-1,4-diazepan-1-yl)butyl)benzofuran-3-ylcarbamide,
N-(4-(4-(2,3-dihydrobenzo[1,4]dioxin-6-yl)-1,4-diazepan-1-yl)butyl)indol-3-ylcarbamide,
N-(4-(4-(3,4-dihydro-2H-benzo[b][1,4]dioxepin-7-yl)piperazin-1-yl)butyl)benzo[b]thiophen-3-ylcarbamide,
N-(4-(4-(3,4-dihydro-2H-benzo[b][1,4]dioxepin-7-yl)piperazin-1-yl)butyl)benzofuran-3-ylcarbamide,
N-(4-(4-(3,4-dihydro-2H-benzo[b][1,4]dioxepin-7-yl)piperazin-1-yl)butyl)indol-3-ylcarbamide, and
physiologically acceptable salts thereof:
(d) a compound of formula VI selected from the group consisting of
N-(4-(4-(2,3-dihydrobenzofuran-5-yl)piperazin-1-yl)butyl)benzo[b]thiophen-2-ylcarbamide,
N-(4-(4-(2,3-dihydrobenzofuran-5-yl)piperazin-1-yl)butyl)-5-cyanobenzo[b]thiophen-2-ylcarbamide,
N-(4-(4-(2,3-dihydrobenzofuran-5-yl)piperazin-1-yl)butyl)benzofuran-2-ylcarbamide,
N-(4-(4-(2,3-dihydrobenzofuran-5-yl)piperazin-1-yl)butyl)-5-bromobenzofuran-2-ylcarbamide,
N-(4-(4-(2,3-dihydrobenzofuran-5-yl)piperazin-1-yl)butyl)indol-2-ylcarbamide,
N-(4-(4-(2,3-dihydrobenzofuran-5-yl)piperazin-1-yl)butyl)-6-cyanoindol-2-ylcarbamide,
N-(4-(4-(chroman-6-yl)piperazin-1-yl)butyl)benzo[b]thiophen-2-ylcarbamide,
N-(4-(4-(chroman-6-yl)piperazin-1-yl)butyl)-5-cyanobenzo[b]thiophen-2-ylcarbamide,
N-(4-(4-(chroman-6-yl)piperazin-1-yl)butyl)benzofuran-2-ylcarbamide,
N-(4-(4-(chroman-6-yl)piperazin-1-yl)butyl)-5-bromobenzofuran-2-ylcarbamide,
N-(4-(4-(chroman-6-yl)piperazin-1-yl)butylindol-2-ylcarbamide,
N-(4-(4-(chroman-6-yl)piperazin-1-yl)butyl)-6-cyanoindol-2-ylcarbamide,
N-(4-(4-(2,3,4,5-tetrahydrobenzo[b]oxepin-7-yl)piperazin-1-yl)butyl)benzo[b]thiophen-2-ylcarbamide,
N-(4-(4-(2,3,4,5-tetrahydrobenzo[b]oxepin-7-yl)piperazin-1-yl)butyl)-5-cyanobenzo[b]thiophen-2-ylcarbamide,
N-(4-(4-(2,3,4,5-tetrahydrobenzo[b]oxepin-7-yl)piperazin-1-yl)butyl)benzofuran-2-ylcarbamide,
N-(4-(4-(2,3,4,5-tetrahydrobenzo[b]oxepin-7-yl)piperazin-1-yl)butyl)-5-bromobenzofuran-2-ylcarbamide,
N-(4-(4-(2,3,4,5-tetrahydrobenzo[b]oxepin-7-yl)piperazin-1-yl)butyl)indol-2-ylcarbamide,
N-(4-(4-(2,3,4,5-tetrahydrobenzo[b]oxepin-7-yl)piperazin-1-yl)butyl)-6-cyanoindol-2-ylcarbamide, and
physiologically acceptable salts thereof: and
(e) a compound of formula VII selected from the group consisting of
N-(4-(4-(2,3-dihydrobenzofuran-5-yl)piperazin-1-yl)butyl)benzo[b]thiophen-3-ylcarbamide,
N-(4-(4-(2,3-dihydrobenzofuran-5-yl)piperazin-1-yl)butyl)benzofuran-3-ylcarbamide,
N-(4-(4-(2,3-dihydrobenzofuran-5-yl)piperazin-1-yl)butyl)indol-3-ylcarbamide,
N-(4-(4-(chroman-6-yl)piperazin-1-yl)butyl)benzo[b]thiophen-3-ylcarbamide,
N-(4-(4-(chroman-6-yl)piperazin-1-yl)butyl)benzofuran-3-ylcarbamide,
N-(4-(4-(chroman-6-yl)piperazin-1-yl)butyl)indol-3-ylcarbamide,
N-(4-(4-(2,3,4,5-tetrahydrobenzo[b]oxepin-7-yl)piperazin-1-yl)butyl)benzo[b]thiophen-3-ylcarbamide,
N-(4-(4-(2,3,4,5-tetrahydrobenzo[b]oxepin-7-yl)piperazin-1-yl)butyl)benzofuran-3-ylcarbamide,
N-(4-(4-(2,3,4,5-tetrahydrobenzo[b]oxepin-7-yl)piperazin-1-yl)butyl)indol-3-ylcarbamide, and
physiologically acceptable salts thereof.

21. A pharmaceutical composition comprising a compound according to claim 1 and at least one pharmaceutically acceptable adjuvant.

22. A pharmaceutical composition comprising a compound according to claim 11 and at least one pharmaceutically acceptable adjuvant.

23. A pharmaceutical composition comprising a compound according to claim 12 and at least one pharmaceutically acceptable adjuvant.

24. A pharmaceutical composition comprising a compound according to claim 19 and at least one pharmaceutically acceptable adjuvant.

25. A pharmaceutical composition comprising a compound according to claim 20 and at least one pharmaceutically acceptable adjuvant.

26. A method of treating a disease state in whose pathogenesis dopaminergic and/or serotoninergic processes are involved, the method comprising administering to a subject in need of such treatment a compound according to claim 1.

27. A method for the treatment of pain or a disorder of the urinary tract in a subject, comprising administering to the subject a compound according to claim 1.

28. A method for treating a central nervous system disease or a urinary tract disorder in a mammal comprising administering to the mammal a compound according to claim 1.

29. The method according to claim 28, wherein the or disorder is selected from psychoses, schizophrenia, anxiety disorders, compulsive disorders, drug dependency, depressive disorders, drug-induced extrapyramidal motor disturbances, Parkinson's disease, Segawa syndrome, Tourette's syndrome, restless leg syndrome, sleeping disorders, nausea, cognitive disorders, male erectile dysfunction, hyperprolactinemia, hyperprolactinomia, glaucoma, attention deficit hyperactive syndrome (ADHS), autism, stroke and urinary incontinence.

30. A process of producing a compound according to claim 1, comprising reacting an acid derivative A 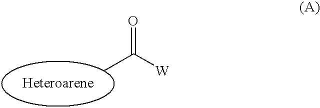
with a free base of general formula C 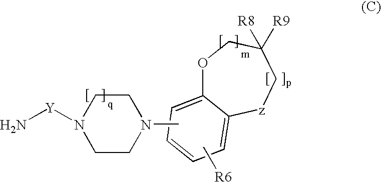
wherein
W is OH, Cl, Br or a group 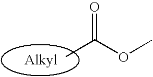
Heteroarene of the acid derivative A is a group of general formula Ia; and 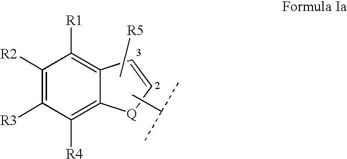
the crossed-through bonding of formula Ia is the bonding of the —C(O)—W group to the 2- or 3-position of the heteroarene with formula Ia.

31. The process according to claim 30, wherein W is chlorine, bromine or OH.

32. The process according to claim 30, wherein W is hydroxy and the acid group is activated by addition of an activation reagent prior to the conversion with the free base of general formula C.

33. The process according to claim 32, wherein the activation reagent is selected from hydroxybenzotriazole, hydroxyazabenzotriazole, HATU and TBTU.

34. A pharmaceutical composition comprising L-dopa or a neuroleptic drug and a compound according to claim 1.

35. A pharmaceutical composition comprising L-dopa or a neuroleptic drug and a compound according to claim 11.

36. A pharmaceutical composition comprising L-dopa or a neuroleptic drug and a compound according to claim 12.

37. A pharmaceutical composition comprising L-dopa or a neuroleptic drug and a compound according to claim 19.

38. A pharmaceutical composition comprising L-dopa or a neuroleptic drug and a compound according to claim 20.
